(19) United States
(12) Patent Application Publication
(10) Pub. No.: US 2017/0306047 A1
(43) Pub. Date: Oct. 26, 2017

(54) PHARMACEUTICAL COMPOSITION FOR CANCER PREVENTION AND TREATMENT, CONTAINING NDRG3 EXPRESSION OR ACTIVITY INHIBITOR AS ACTIVE INGREDIENT, OR NDRG3 PROTEIN-SPECIFIC ANTIBODY AND USE THEREOF

(71) Applicant: KOREA RESEARCH INSTITUTE OF BIOSCIENCE AND BIOTECHNOLOGY, Daejeon (KR)

(72) Inventors: Young II YEOM, Daejeon (KR); Dong Chul LEE, Daejeon (KR); Kyung Chan RARK, Daejeon (KR); Hyun Ahm SOHN, Daejeon (KR); Min Ho KANG, Daejeon (KR)

(73) Assignee: KOREA RESEARCH INSTITUTE OF BIOSCIENCE AND BIOTECHNOLOGY, Daejeon (KR)

(21) Appl. No.: 15/314,242

(22) PCT Filed: Jul. 28, 2014
(86) PCT No.: PCT/KR2014/006873

§ 371 Date: Jun. 12, 2017

(30) Foreign Application Priority Data
May 26, 2014 (KR) 10-2014-0062966
May 26, 2014 (KR) 10-2014-0062986
May 26, 2014 (KR) 10-2014-0063002
May 26, 2014 (KR) 10-2014-0063014

(51) Int. Cl. C07K 16/32; A61K 39/395; C12N 15/113; A61K 31/713; A61K 45/06; A01K 67/027

(52) U.S. Cl. CPC C07K 16/32; A61K 45/06; A01K 67/0278; C12N 15/1135; A61K 31/713; A61K 39/39558; A01K 2267/0331; A01K 2227/105; A01K 2217/052; C07K 2317/34; C07K 2317/76; C12N 2310/14; C12N 2310/531; C12N 2320/31; C07K 2317/20

(57) ABSTRACT

The present invention relates to a pharmaceutical composition for preventing and treating cancer or inflammatory disease, containing an NDRG3 expression or activity inhibitor as an active ingredient. Furthermore, the present invention relates to an NDRG3 protein-specific antibody and a use thereof. Specifically, the antibody to the NDRG3 protein is prepared, and the antibody is used to verify that NDRG3 mediated by lactate generated from a hypoxia reaction promotes cell proliferation, angiogenesis, and cytokine expression through the lactate-NDRG3-c-Raf-ERK signaling pathway, and thus the antibody binding to the epitope of the NDRG3 or a fragment of the antibody can be favorably used in the research of cancer or inflammation occurrence mechanism, the development of novel genes involved in the mechanism, and the development of therapeutic agents and new pharmaceuticals.

### TECHNICAL FIELD

The present invention relates to a pharmaceutical composition for preventing and treating a cancer or inflammatory disease comprising an NDRG3 expression or activity inhibitor as an active ingredient. Also, the present invention relates to an NDRG3 protein-specific antibody and the use thereof.

### BACKGROUND ART

Oxygen homeostasis is essential for metazoan physiology. Under low-oxygen conditions (hypoxic conditions), cells induce hypoxic responses to adapt to and survive harsh environments. Hypoxic responses are mediated by a variety of genes which functions in various biological processes including metabolic adaptation, up-regulation of oxygen carriers, maintenance of pH homeostasis, and stimulation of angiogenesis (Harris, A. L., Nat. Rev. Cancer, 2002(2), 38-47, Cassavaugh, J. & Lounsbury. K. M., J. Cell. Biochem, 2011(112), 735-744).

In general, angiogenesis is known to play a critical role in the growth of solid cancer or cancer metastasis. That is, cancer cells increases the blood supply into tumor by promoting formation of blood vessels through a series of processes called an ‘angiogenic switch’ in which the expression of angiogenic factors is increased and the expression of angiostatic factors are reduced. However, vascular tissues in tumor tissues are usually abnormal tissues, and do not have a relatively sufficient flood flow rate, compared to rapid growth of cancer mass, results in hypoxia in which the oxygen supply runs short relative to the oxygen demand in cancer cells during a progress of cancer. Also, the expression of various types of genes involved in angiogenesis, iron metabolism, glucose metabolism, and cell proliferation and survival is induced to cope with a low partial pressure of oxygen. That is, when the partial pressure of oxygen is insufficient, a series of intracellular changes associated with hypoxia occurs. Among theses, hypoxia-inducible factor-1 (HIF-1) is the most representative one of transcription activators that responds to the partial pressure of oxygen, and thus regulates various genes involved in the hypoxia (Clinical and Molecular Hepatology, Volume 13, Issue 1, 2007, Pages 9-19). In particular, misregulation of hypoxia or HIF-1 and its target signals is associated with the bad prognosis of cancer patients as well as the failure of cancer treatment (Semenza, G. L., Cancer Cell, 2004(5), 405-406, Welsh, S. J. et al., Semin. Oncol, 2006(33), 486-497).

All types of biological defense responses made to restore the structures and functions of damaged tissues caused by infections, trauma, etc. are generally referred to inflammatory responses. Mobilization of white blood cells into affected areas is important for quickly resolving infections and restoring tissue damage caused by various traumas. However, wrong or sustained inflammatory responses cause human tissue damage and diseases. For example, inflammatory diseases are caused by bacterial or viral infections such as cerebrospinal meningitis, enteritis, dermatitis, uveitis, encephalitis, adult respiratory distress syndrome, etc., or non-inflammatory factors such as trauma, autoimmune diseases, organ xenograft rejection, etc. The inflammatory diseases are divided into acute and chronic inflammatory diseases whose symptoms or pathologic characteristics distinguish from each other. Local symptoms of acute inflammations such as allergy, and bacterial or viral infection occur due to a blood flow change, a change in size of blood vessels, a change in vascular permeability, and infiltration of white blood cells, etc. On the other hand, one main pathologic characteristic of chronic inflammations including rheumatoid arthritis, atheriosclerosis, chronic nephritis, liver cirrhosis, and the like is to continuously infiltrate monocytes, neutrophils, lymphocytes and plasmocytes into sites of inflammation since pro-inflammatory factors are not removed, resulting in chronic inflammatory responses.

Inflammatory mediators expressed at sites of inflammation, that is, cytokines, chemokines, reactive oxygen intermediates, cycloxygenase-2 (COX-2), 5-lipoxygenase (5-LOX), matrix metalloproteinase (MMP), and the like play important roles in generation and maintenance of inflammatory responses. The expression of such inflammatory mediators is known to be regulated by transcription factors such as nuclear factor κB (NF-κB), signal transducer and activator of transcription 3 (STAT3), activator protein 1 (AP-1), hypoxia-inducible factor 1a (HIF-1a), etc.

Hypoxia-inducible factor-1 (HIF-1) is a nuclear transcription factor that is induced to be expressed at a large quantity under a hypoxic condition, and functions to express genes associated with erythropoiesis, angiogenesis and glycolysis so as to maintain oxygen homeostasis in cells. HIF-1 is subdivided into two groups HIF-1α and 1β. Here, HIF-1α is a transcription factor that is degraded at a normal partial pressure of oxygen, but this protein itself is known to be stabilized under a hypoxic condition. The stabilized HIF-1α binds to HIF-1β such as ARNT to move into the nuclei and expresses genes associated with angiogenesis and metabolisms (Semenza et al., 1999; Wang et al., 1995; Wang and Semenza 1995). Since the activity of HIF-1 is closely associated with cancer development and metastasis, and pathologic mechanisms of various chronic metabolic diseases such as rheumatoid arthritis, ischemic stroke, arteriosclerosis, etc., it has emerged as a new drug target. In particular, HIFs has been used as a therapeutic target to treat cancer with the highest priority since they play an important role in hypoxic responses. However, it has been reported that the progression of cancer induced by the hypoxic responses cannot be completely prevented through inhibition of HIFs, and a regulatory pathway induced in a HIF-independent manner is induced in this way. Therefore, there is a need for strategy in which HIFs are used in combination of inhibitory materials for factors associated with the HIF-independent regulatory pathway to treat cancers or various chronic metabolic diseases.

An N-myc downstream-regulated gene (NDRG) family consists of genes whose expression increases in N-Myc mutant mice, and was first found under the name of ‘Ndr1.’ The NDRG family has started to be named Drg1, Cap43, RTP/rit42, and the like since its human ortholog NDRG1 was isolated from a human cell line. The NDRG family was reported to consist of four different types of constitutive genes. Also, it was reported that the four types of the constitutive genes, such as NDRG1, NDRG2, NDRG3 and NDRG4, have high homology, but has very different expression patterns with the development and growth of individuals (Qu et al., Mol Cell Biochem, 2002(229), 35-44, Deng et al., Int J Cancer, 2003(106), 342-7). Therefore, these genes are expected to have different functions in terms of a difference in such expression, but there are no reports on known functions of the genes.

Accordingly, the present inventors have made an effort to find HIF-independent factors associated with hypoxia in order to treat cancer or inflammations, and found that an antibody against an NDRG3 protein is prepared, and NDRG3 mediated by lactate generated from a hypoxic response and mediated in an HIF-independent manner promotes expression of cytokines, which mediates cell proliferation, angiogenesis and inflammatory response through a lactate-NDRG3-c-Raf-ERK signaling pathway, using the antibody, and thus an inhibitor for inhibiting expression or activity of the NDRG3 may be usefully used as a pharmaceutical composition for preventing and treating cancer or inflammations. Therefore, the present invention has been completed based on these facts.

Also, the present inventors have made an effort to find HIF-independent factors associated with hypoxia, and found that an antibody against an NDRG3 protein is prepared, and is used to verify that the expression and activity of NDRG3 are enhanced by lactate generated from a hypoxic response and the NDRG3 mediated by the lactate promotes cell proliferation and angiogenesis through a c-Raf-ERK signaling pathway, and thus the NDRG3 protein-specific antibody may be usefully used to conduct research on onset mechanisms of diseases, such as cancer or inflammatory diseases, caused by hypoxia, screen novel genes involved in the onset mechanisms, and develop therapeutic agents and new pharmaceuticals. Therefore, the present invention has been completed based on these facts.

Further, the present inventors have made an effort to develop a disease model involved in hypoxia, and found that tumor is formed in tissues from liver, intestines and lungs of a transgenic mouse prepared to overexpress NDRG3, and angiogenesis and cytokine expression increases in the liver tissues, and thus the NDRG3-overexpressing transgenic mouse model may be usefully used an animal model of diseases to conduct onset mechanisms of diseases, such as cancer or inflammatory diseases, caused by hypoxia, screen novel genes involved in the onset mechanisms, and develop therapeutic agents and new pharmaceuticals. Therefore, the present invention has been completed based on these facts.

### DISCLOSURE

### Technical Problem

Therefore, the present invention is designed to solve the problems of the prior art, and it is an object of the present invention to provide a pharmaceutical composition for preventing and treating a cancer comprising an N-myc downstream-regulated gene 3 (NDRG3) expression or activity inhibitor as an active ingredient.

It is another object of the present invention to provide a pharmaceutical composition for preventing and treating inflammatory disease comprising an NDRG3 expression or activity inhibitor as an active ingredient.

It is still another object of the present invention to provide an NDRG3 protein-specific antibody and a use thereof.

It is yet another object of the present invention to provide an NDRG3-overexpressing transgenic animal model and a use thereof.

### Technical Solution

To solve the above problems, according to an aspect of the present invention, there is provided a pharmaceutical composition for preventing and treating a cancer comprising an N-myc downstream-regulated gene 3 (NDRG3) expression or activity inhibitor as an active ingredient.

According to another aspect of the present invention, there is provided a pharmaceutical composition for preventing and treating a cancer comprising an NDRG3 protein expression or activity inhibitor and a hypoxia-inducible factor (HIF) inhibitor as active ingredients.

According to still another aspect of the present invention, there is provided a method of detecting an NDRG3 protein to provide information on cancer comprising:

1) measuring expression or activity of an NDRG3 protein from a specimen isolated from a test object; and

2) determining to have cancer or be at risk of having cancer when the expression or activity of the NDRG3 protein in step 1 is increased compared to the normal control.

According to yet another aspect of the present invention, there is provided a method of screening a pharmaceutical composition for preventing and treating a cancer comprising:

1) treating an NDRG3 protein-expressing cell line with a test material;

2) confirming expression or activity of an NDRG3 protein in the cell line of step 1; and

3) selecting the test material that reduces the expression or activity of the NDRG3 protein in step 2, compared to the untreated control.

According to yet another aspect of the present invention, there is provided a method of screening a pharmaceutical composition for preventing and treating a cancer comprising:

1) treating a cell line, which expresses NDRG3 and one or more selected from PKC-β, RACK1 and c-Raf, with a test material under a hypoxic condition;

2) determining a binding degree of the NDRG3 to one or more selected from the PKC-β, RACK1 and c-Raf in the cell line of step 1; and

3) selecting the test material that reduces the binding degree in step 2, compared to the untreated control.

According to yet another aspect of the present invention, there is provided a method of screening a pharmaceutical composition for preventing and treating a cancer comprising:

1) treating NDRG3, PKC-β, RACK1 and c-Raf proteins with a test material in vitro;

2) determining a binding degree of one or more selected from the NDRG3, PKC-β, RACK1 and c-Raf proteins in step 1; and

3) selecting the test material which reduces the binding degree in step 2, compared to the untreated control.

According to yet another aspect of the present invention, there is provided a pharmaceutical composition for preventing and treating an inflammatory disease comprising an NDRG3 expression or activity inhibitor as an active ingredient.

According to yet another aspect of the present invention, there is provided a method of detecting an NDRG3 protein to provide information on diagnosis of an inflammatory disease comprising:

1) measuring expression or activity of an NDRG3 protein from a specimen isolated from a test object; and

2) diagnosing to have the inflammatory disease or predicting to be at risk of having the inflammatory disease when the expression or activity of the NDRG3 protein in step 1 is increased compared to the normal control.

According to yet another aspect of the present invention, there is provided a method of screening a pharmaceutical composition for preventing and treating an inflammatory disease comprising:

1) treating an NDRG3 protein-expressing cell line with a test material;

2) confirming expression or activity of the NDRG3 protein in the cell line of step 1; and

3) selecting the test material which reduces the expression or activity of the NDRG3 protein in step 2, compared to the untreated control.

According to yet another aspect of the present invention, there is provided a method of screening a pharmaceutical composition for preventing and treating an inflammatory disease comprising:

1) treating a cell line, which expresses NDRG3 and one or more selected from PKC-β, RACK1 and c-Raf, with a test material under a hypoxic condition;

2) determining a binding degree of the NDRG3 to one or more selected from the PKC-β, RACK1 and c-Raf in the cell line of step 1; and

3) selecting the test material which reduces the binding degree in step 2, compared to the untreated control.

According to yet another aspect of the present invention, there is provided a method of screening a pharmaceutical composition for preventing and treating an inflammatory disease comprising:

1) treating NDRG3, PKC-β, RACK1 and c-Raf proteins with a test material in vitro;

2) determining a binding degree of one or more selected from the NDRG3, PKC-β, RACK1 and c-Raf proteins in step 1; and

3) selecting the test material which reduces the binding degree in step 2, compared to the untreated control.

According to yet another aspect of the present invention, there is provided an antibody or an immunologically active fragment thereof which specifically binds to an NDRG3 epitope.

According to yet another aspect of the present invention, there is provided a composition comprising an antibody or immunologically active fragment thereof which specifically binds to the NDRG3 epitope.

According to yet another aspect of the present invention, there is provided a pharmaceutical composition for preventing and treating a cancer or inflammatory disease comprising an antibody or immunologically active fragment thereof which specifically binds to the NDRG3 epitope.

According to yet another aspect of the present invention, there is provided a kit for diagnosing a cancer or inflammatory disease comprising treating a test specimen with an antibody or immunologically active fragment thereof which specifically binds to the NDRG3 epitope.

According to yet another aspect of the present invention, there is provided an NDRG3-overexpressing transgenic mouse that is transfected with a vector comprising a promoter, an NDRG3 gene, and a polyadenylation sequence.

According to yet another aspect of the present invention, there is provided a fertilized egg from a transgenic mouse obtained by injecting a vector comprising a promoter, an NDRG3 gene, and a polyadenylation sequence, into a fertilized egg of a mouse.

According to yet another aspect of the present invention, there is provided a method of preparing an NDRG3-overexpressing transgenic mouse comprising:

1) micro-injecting a vector comprising a promoter, an NDRG3 gene, and a polyadenylation sequence, into a fertilized egg of a mouse;

2) transplanting the fertilized egg into an oviduct to obtain littermates; and

3) determining whether injected DNA is inserted to select a founder mouse from the littermates.

According to yet another aspect of the present invention, there is provided a method of screening a pharmaceutical composition for preventing and treating a cancer or inflammatory disease comprising:

1) treating the NDRG3-overexpressing transgenic mouse with a candidate material;

2) confirming expression or activity of a NDRG3 protein in a specimen derived from the NDRG3-overexpressing transgenic mouse in step 1; and

3) selecting the candidate material which reduces the expression or activity of the NDRG3 protein in step 2, compared to that in tissues from an untreated control mouse.

According to yet another aspect of the present invention, there is provided a method of preventing a cancer comprising administering a pharmaceutically effective amount of the NDRG3 protein expression or activity inhibitor to a subject.

According to yet another aspect of the present invention, there is provided a method of treating a cancer comprising administering a pharmaceutically effective amount of the NDRG3 protein expression or activity inhibitor to a subject.

According to yet another aspect of the present invention, there is provided a method of preventing a cancer comprising administering pharmaceutically effective amounts of the NDRG3 protein expression or activity inhibitor and the HIF inhibitor to a subject.

According to yet another aspect of the present invention, there is provided a method of treating a cancer comprising administering pharmaceutically effective amounts of the NDRG3 protein expression or activity inhibitor and the HIF inhibitor to a subject.

According to yet another aspect of the present invention, there is provided a method of preventing an inflammatory disease comprising administering a pharmaceutically effective amount of the NDRG3 protein expression or activity inhibitor to a subject.

According to yet another aspect of the present invention, there is provided a method of treating an inflammatory disease comprising administering a pharmaceutically effective amount of the NDRG3 protein expression or activity inhibitor to a subject.

According to yet another aspect of the present invention, there is provided a method of preventing a cancer or inflammatory disease comprising administering a pharmaceutically effective amount of the antibody or immunologically active fragment thereof, which specifically binds to the NDRG3 epitope, to a subject.

According to yet another aspect of the present invention, there is provided a method of treating a cancer or inflammatory disease comprising administering a pharmaceutically effective amount of the antibody or immunologically active fragment thereof, which specifically binds to the NDRG3 epitope, to a subject.

According to yet another aspect of the present invention, there is provided a NDRG3 protein expression or activity inhibitor for use as a pharmaceutical composition for preventing and treating a cancer.

According to yet another aspect of the present invention, there is provided a NDRG3 protein expression or activity inhibitor and a HIF inhibitor for use as a pharmaceutical composition for preventing and treating a cancer.

According to yet another aspect of the present invention, there is provided a NDRG3 protein expression or activity inhibitor for use as a pharmaceutical composition for preventing and treating an inflammatory disease.

According to yet another aspect of the present invention, there is provided an antibody or an immunologically active fragment thereof, which specifically binds to the NDRG3 epitope, for use as a pharmaceutical composition for preventing and treating a cancer or inflammatory disease.

### Advantageous Effects

The NDRG3 protein according to the present invention has an increased expression and activity by lactate generated from a sustained hypoxic response, and binds to c-Raf and RACK1 by interacting with a scaffold protein through the increased expression and activity. In this case, the NDRG3 protein can recruit PKC proteins through RACK1 to form a single complex consisting of the four materials, and promote expression of cytokines which activates a c-Raf-ERK signaling pathway via the single complex to mediate cell proliferation, angiogenesis and inflammatory response. Therefore, an inhibitor for inhibiting expression or activity of the NDRG3 protein can be usefully used as a pharmaceutical composition for preventing and treating a cancer or inflammatory disease. Also, an antibody or a fragment thereof specifically binding to the NDRG3 epitope can be usefully used to conduct research on onset mechanisms of diseases, such as cancers, inflammations, blood vessel diseases, and the like, caused by hypoxia, screen novel genes involved in the onset mechanisms, and develop therapeutic agents and new pharmaceuticals.

### DESCRIPTION OF DRAWINGS

FIG. 1A is a diagram showing a pCAGGS plasmid that codes for human NDRG3 to prepare an NDRG3-overexpressing transgenic mouse.

FIG. 1B is a diagram showing whether a human NDRG3 gene is inserted into genomic DNAs of TG-2, TG-8 and TG-13 mice, which are in a process of producing an NDRG3-overexpressing transgenic mouse.

FIG. 1C is a diagram showing that the human NDRG3 gene is expressed in liver tissues from the established NDRG3-overexpressing transgenic mice TG-2, TG-8 and TG-13.

FIG. 2 is diagram showing an antigen-antibody reaction between a prepared rabbit anti-NDRG3 antibody and a human NDRG3 (N66D) variant.

FIG. 3A is a diagram schematically showing a process of selecting a candidate protein binding to PHD2.

FIG. 3B is a diagram showing a protein binding to PHD2.

FIG. 3C is a diagram showing binding of PHD2 and NDRG3 proteins.

FIG. 4A is a diagram showing binding of the PHD2 and NDRG3 under a hypoxic condition (1% O₂; Upper panel of FIG. 4A) and in vitro (Lower panel of FIG. 4A).

FIG. 4B is a diagram showing that the NDRG3 protein is induced by inhibiting PHD2 in MCF-7 cells which are present under a normal oxygen condition (normoxic condition; 21% O₂).

FIG. 4C is a diagram showing that the NDRG3 protein is induced by inhibiting PHD2 in HeLa cells which are present under the normoxic condition.

FIG. 5A is a diagram showing that expression of the NDRG3 protein is inhibited by deletion of a PHD family group and VHL under a normoxic condition.

FIG. 5B is a diagram showing binding of the PHD family group and the NDRG3 protein.

FIG. 6A is a diagram showing a PHD2 docking site of NDRG3 through a protein-protein docking simulation.

FIG. 6B is a diagram showing a binding affinity of PHD2 to the PHD2 docking site of the NDRG3 confirmed through the docking simulation.

FIG. 7A is a diagram showing the NDRG3 protein ubiquitinated after treated with a proteasome inhibitor MG132 under a normoxic condition.

FIG. 7B is a diagram showing the NDRG3 protein ubiquitinated under the normoxic condition.

FIG. 8A is a diagram showing the NDRG3 protein accumulated under a sustained hypoxic condition.

FIG. 8B is a diagram showing the NDRG3 protein accumulated in cells under the sustained hypoxic condition.

FIG. 8C is a diagram showing the NDRG3 protein accumulated in various types of cancer cells under the sustained hypoxic condition.

FIG. 8D is a diagram showing that the ubiquitination of the NDRG3 protein is inhibited under the sustained hypoxic condition.

FIG. 9A is a diagram showing changes in expression of the NDRG3 protein under the normoxic condition and the sustained hypoxic condition.

FIG. 9B is a diagram showing changes in expression of the NDRG3 protein when the hypoxic condition returns to the normoxic condition.

FIG. 10A is a diagram showing a hydroxylation target site of the NDRG3 protein.

FIG. 10B is a diagram showing the hydroxylation target site of the NDRG3 protein and binding of PHD2/VHL.

FIG. 11A is a diagram showing changes in expression of RNA of NDRG3 and the HIF protein in response to a change in oxygen conditions.

FIG. 11B is a diagram showing a change in expression of the NDRG3 protein in response to inhibition of HIF and PHD2.

FIG. 11C is a diagram showing changes in expression of the NDRG3 protein in MCF-7 (HIF-1^{+/+} and VHL^{+/+}) and 786-O (HIF-1^{-/-} and VHL^{-/-}) cells, which are present under the hypoxic condition.

FIG. 12A is a diagram showing analyses of functions of NDRG3 and HIF-1 associated with a hypoxic response.

FIG. 12B is a diagram showing analyses of functions of genes which are up-regulated in cells that overexpress the NDRG3 protein under the normoxic condition and cells that are present under the hypoxic condition.

FIG. 13A is a diagram showing a change in angiogenesis activity in response to NDRG3 deletion under the hypoxic condition.

FIG. 13B is a diagram showing changes in expression of angiogenic markers IL8, IL1α, IL1β, COX-2 and PAI-1 in response to the NDRG3 deletion under the hypoxic condition.

FIG. 13C is a diagram showing a change in in vivo angiogenesis in response to the NDRG3 deletion.

FIG. 14A is a diagram showing a change in cell growth in response to the NDRG3 deletion.

FIG. 14B is a diagram showing a change in tumorigenesis in response to NDRG3 and/or HIF deletion.

FIG. 14C is a diagram showing a change in tumorigenesis in response to expression of an ectopic variant NDRG3 (N66D).

FIG. 14D is a diagram showing a change in volume of tumor in a mouse transplanted with NDRG3- and/or HIF-deleted tumor cells.

FIG. 14E is a diagram showing a change in volume of tumor in a mouse transplanted with ectopic variant NDRG3 (N66D)-expressed tumor cells.

FIG. 14F is a diagram showing changes in expression of a cell proliferation marker Ki-67 and an angiogenic marker IL8 protein in response to NDRG3 or HIF deletion in tumor tissues.

FIG. 14G is a diagram showing a change in expression of the angiogenic marker in response to the NDRG3 or HIF deletion in the tumor tissues.

FIG. 15A is a diagram showing a change in expression of NDRG3 and HIF-1α proteins and a change in generation of lactate in response to the oxygen conditions.

FIG. 15B is a diagram showing a change in expression of the NDRG3 and HIF-1α proteins and a change in generation of lactate in response to the sustained hypoxic condition after treatment with sodium oxamate that is a lactate dehydrogenase A (LDHA) inhibitor.

FIG. 15C is a diagram showing a change in expression of the NDRG3 protein in response to the generation of lactate under the hypoxic condition.

FIG. 15D is a diagram showing a change in expression of the NDRG3 protein in response to glycolysis by 2-deoxyglucose (2-DG).

FIG. 15E is a diagram showing a change in expression of the NDRG3 protein in response to the excessive generation of lactate by pyruvate or LDHA.

FIG. 15F is a diagram showing a change in ubiquitination of the NDRG3 protein in response to the generation of lactate.

FIG. 16A is a diagram showing a recombinant NDRG3 (G138W) variant protein in which a lactate binding site of the NDRG3 protein is mutated, and a wild-type NDRG3 protein.

FIG. 16B is a diagram showing binding of lactate to the wild-type NDRG3 protein and the recombinant NDRG3 (G138W) variant protein.

FIG. 16C is a diagram showing a change in expression of a variant in which an L-lactate binding site of NDRG3 is mutated under a hypoxic condition.

FIG. 16D is a diagram showing the expression of the NDRG3 protein in response to reoxygenation.

FIG. 17A is a diagram showing a change in cell growth in response to inhibition of lactate generation and expression of the ectopic variant NDRG3 (N66D).

FIG. 17B is a diagram showing a change in formation of cell colonies in response to the inhibition of lactate generation and/or the expression of the ectopic variant NDRG3 (N66D).

FIG. 17C is a diagram showing a change in cell growth in response to LDHA deletion and/or the expression of the ectopic variant NDRG3 (N66D).

FIG. 17D is a diagram showing a change in tumorigenesis in a mouse transplanted with tumor cells in which LDHA is deleted and/or the ectopic variant NDRG3 (N66D) is expressed.

FIG. 17E is a diagram showing a change in expression of the NDRG3 protein in response to the inhibition of lactate generation and/or the expression of the ectopic variant NDRG3 (N66D) under a sustained hypoxic condition.

FIG. 17F is a diagram showing a change in the generation of lactate in response to the expression of the ectopic variant NDRG3 (N66D) under the sustained hypoxic condition.

FIG. 18 is a diagram showing a change in angiogenesis in response to the inhibition of lactate generation and/or the expression of the ectopic variant NDRG3 (N66D) under the sustained hypoxic condition.

FIG. 19A is a diagram showing changes in phosphorylation of proteins in response to NDRG3 deletion under a hypoxic condition.

FIG. 19B is a diagram showing a change in activity of an ERK1/2 protein in response to expression of the NDRG3 protein.

FIG. 19C is a diagram showing a change in Raf-ERK1/2 activity in response to NDRG3 deletion under a sustained hypoxic condition.

FIG. 19D is a diagram showing binding of the NDRG3 protein and c-Raf in vitro (Left panel) and in cells (Right panel).

FIG. 19E is a diagram showing a change in Raf-ERK1/2 activity in response to deletion of the NDRG3 protein or expression of the ectopic variant NDRG3 (N66D).

FIG. 19F is a diagram showing a change in phosphorylation of c-Raf in response to inhibition of the PKC-β activity by the ectopic variant NDRG3 (N66D) and/or PKC-I.

FIG. 19G is a diagram showing changes in expression of the NDRG3 protein and Raf-ERK1/2 activity in response to the inhibition of lactate generation under a hypoxic condition.

FIG. 20A is a diagram showing binding between the NDRG3 protein and RACK-1.

FIG. 20B is a diagram showing a change in Raf-ERK1/2 activity in response to NDRG3 deletion or expression of the ectopic variant NDRG3 (N66D), RACK-1 and/or Raf.

FIG. 20C is a diagram showing binding between the ectopic variant NDRG3 (N66D) and PKC-β in response to RACK1 deletion under the hypoxic condition.

FIG. 20D is a diagram showing a change in ERK1/2 activity in response to inhibition of the PKC-β activity under the hypoxic condition.

FIG. 20E is a diagram showing a complex formed between the recombinant ectopic variant NDRG3 (N66D) and c-Raf, RACK1 and PKC-β.

FIG. 20F is a diagram showing the complex of NDRG3, c-Raf, RACK1 and PKc-β through a simulation.

FIG. 21A is a diagram showing the presence of tumorigenesis in NDRG3-overexpressing transgenic mice and a control mouse.

FIG. 21B is a diagram showing the presence of tumorigenesis in lungs, intestines and hypogastria from the NDRG3-overexpressing transgenic mice and the control mouse.

FIG. 21C is a diagram showing B cells and T cells in mesenteric lymph nodes, spleens and liver tissues from the NDRG3-overexpressing transgenic mice and the control mouse.

FIG. 21D is a diagram showing expression of hepatocellular carcinoma (HCC) markers, glutamine synthetase (GS) and heat shock protein 70 (HSP70), and cell proliferation markers, PCNA and Ki-67, in the liver tissues from the NDRG3-overexpressing transgenic mice and the control mouse.

FIG. 21E is a diagram showing expression of mRNAs of ERK1/2 activity and angiogenic markers in the liver issues from the NDRG3-overexpressing transgenic mice and the control mouse.

FIG. 22 is a diagram showing expression of NDRG3 and ERK1/2 activity in a liver cancer patient.

FIG. 23 is a diagram illustrating a mechanism of NDRG3 as a mediator of a Raf-ERK pathway induced by lactate in a sustained hypoxic response.

### BEST MODE

Hereinafter, the present invention will be described in detail.

The present invention provides a pharmaceutical composition for preventing and treating a cancer comprising an N-myc downstream-regulated gene 3 (NDRG3) protein expression or activity inhibitor as an active ingredient.

The NDRG3 protein preferably consists of an amino acid sequence set forth in SEQ ID NO: 1.

The NDRG3 protein expression inhibitor preferably includes at least one selected from the group consisting of antisense nucleotide, short interfering RNA (siRNA), and short hairpin RNA (shRNA) sequences, all of which complementarily bind to mRNA of an NDRG3 gene, but the present invention is not limited thereto.

The antisense nucleotide binds (hybridizes) to a complementary base sequence of DNA, immature mRNA or mature mRNA to interfere with a flow of genetic information from DNA to a protein, as defined in the Watson-Crick base pair. The nature of the antisense nucleotide having specificity to target sequences serves to exceptionally multi-functionalize the target sequences. Since the antisense nucleotide has a long chain of monomer units, the antisense nucleotide may be easily synthesized for target RNA sequences. Many recent studies have verified the utility of the antisense nucleotide as a biochemical means for research of target proteins (Rothenberg et al., J. Natl. Cancer Inst., 81:1539-1544, 1999). In recent years, there are many advances in the fields of oligonucleotide chemistry and synthesis of nucleotides showing improved cell adhesion, target binding affinity and nuclease resistance. Thus, the antisense nucleotide may be considered to be used as a new type of an inhibitor.

In the siRNA, a sense RNA is combined with an antisense RNA to form a double-stranded RNA molecule. In this case, the sense RNA is a preferably an siRNA molecule including the same nucleic acid sequence as a target sequence of a portion of the consecutive nucleotide in the NDRG3 mRNA. The siRNA molecule preferably consists of a sense sequence consisting of 10 to 30 bases selected from a base sequence of an NDRG3 gene, and an antisense sequence complementarily binding to the sense sequence, but the present invention is not limited thereto. For example, any double-stranded RNA molecules having a sense sequence that may complementarily bind to the base sequence of the NDRG3 gene may be used herein. The antisense sequence most preferably has a sequence complementary to the sense sequence.

The NDRG3 protein expression inhibitor preferably promotes hydroxylation a proline residue at position 294 of the NDRG3 protein, but the present invention is not limited thereto. In one exemplary embodiment of the present invention, it was confirmed that accumulation of an NDRG3 variant, in which the 294^{th} proline of NDRG3 is substituted with alanine even under a normoxic condition, increases, and interaction of the NDRG3 variant with PHD2/VHL decreases. As a result, it is revealed that the 294^{th} proline of NDRG3 is a site for inhibiting expression of the NDRG3 protein.

The NDRG3 protein expression inhibitor preferably promotes binding of PHD2 to one or more PHD2 docking sites selected from the group consisting of 47^{th} arginine, 66^{th} asparagine, 68^{th} lysine, 69^{th} serine, 72^{nd} asparagine, 73^{rd} alanine, 76^{th} asparagine, 77^{th} phenylalanine, 78^{th} glutamic acid, 81^{st} glutamine, 97^{th} glutamine, 98^{th} glutamine, 99^{th} glutamic acid, 100^{th} glycine, 101^{st} alanine, 102^{nd} proline, 103^{rd} serine, 203^{rd} leucine, 204^{th} aspartic acid, 205^{th} leucine, 208^{th} threonine, 209^{th} tyrosine, 211^{th} methionine, 212^{th} histidine, 214^{th} alanine, 215^{th} glutamine, 216^{th} aspartic acid, 217^{th} isoleucine, 218^{th} asparagine, 219^{th} glutamine, 296^{th} valine, 297^{th} valine, 298^{th} glutamine, 300^{th} glycine, and 301^{st} lysine of the NDRG3 protein, more preferably promotes the binding of PHD2 to one or more PHD2 docking sites selected from the group consisting of 47^{th} arginine, 66^{th} asparagine, 68^{th} lysine, 69^{th} serine, 97^{th} glutamine, and 296^{th} valine of the NDRG3 protein, and most preferably promotes the binding of PHD2 to one or more PHD2 docking sites selected from the group consisting of 47^{th} arginine, 66^{th} asparagine, and 68^{th} lysine of the NDRG3 protein, but the present invention is not limited thereto.

The NDRG3 protein expression inhibitor preferably inhibits binding of lactate to 62^{nd} aspartic acid, 138^{th} glycine, 139^{th} alanine, or 229^{th} tyrosine which is a lactate binding site of the NDRG3 protein, but the present invention is not limited thereto.

The NDRG3 protein activity inhibitor is preferably an aptamer or antibody that complementarily binds to the NDRG3 protein, but the present invention is not limited thereto.

In the case of the NDRG3 protein activity inhibitor, the aptamer itself complementarily binding to the NDRG3 protein is a single-stranded nucleic acid (DNA, RNA or modified nucleic acids) that has a stable three-dimensional (3D) structure, and also has a characteristic of specifically binding to target molecules with high affinity. The aptamer was first found using an aptamer screening technique called “Systematic Evolution of Ligands by EXponential enrichment (SELEX)” (Ellington, A D and Szostak, J W., Nature 346:818-822, 1990). Since then, many aptamer, such as low-molecular organic substances, peptides, membrane proteins, and the like, which may bind to various target molecules, have been continuously screened. Since the aptamer has characteristics of having high intrinsic affinity (generally a pM level) and specifically binding to the target molecules, the aptamer is often comparable with a single antibody, and especially has a high probability as an alternative antibody which is referred to as a “chemical antibody.”

All types of antibodies prepared by injecting NDRG3 or purchased on market may be used as the antibody complementarily binding to the NDRG3 protein. Also, the antibody includes a polyclonal antibody, a monoclonal antibody, and fragment thereof capable of binding to epitopes. The polyclonal antibody may be produced using a conventional method which includes injecting NDRG3 into an animal and gathering blood from the corresponding animal to obtain sera including the antibody. Such a polyclonal antibody may be purified using any methods known in the related art, and may be prepared from any animal species host such as a goat, a rabbit, a sheep, a monkey, a horse, a pig, cattle, a dog, etc. The monoclonal antibody may be prepared using any techniques of continuously culturing a cell line to provide production of antibody molecules by continuously culturing a cell line. Such techniques include a hybridoma technique, a human B-cell hybridoma technique, and an EBV-hybridoma technique, but the present invention is not limited thereto (Kohler G et al., Nature 256:495-497, 1975; Kozbor D et al., J Immunol Methods 81:31-42, 1985; Cote R J et al., Proc Natl Acad Sci 80:2026-2030, 1983; and Cole S P et al., Mol Cell Biol 62:109-120, 1984). Also, the antibody fragment comprising a certain binding site to the NDRG3 protein may be prepared. For example, an F(ab′)2 fragment may be prepared by decomposing an antibody molecule with pepsin, and an Fab fragment may be prepared by reducing a disulfide bridge of the F(ab′)2 fragment, but the present invention is not limited thereto. As an alternative method, an Fab expression library may be constructed at a small scale, by which a monoclonal Fab fragment having desired specificity may be identified in a fast and easy manner (Huse W D et al., Science 254: 1275-1281, 1989).

The antibody may be bound to a solid substrate to promote subsequent processes such as washing or complex separation. The solid substrate, for example, includes a synthetic resin, nitrocellulose, a glass substrate, a metal substrate, glass fibers, microspheres, and microbeads. Also, the synthetic resin includes a polyester, polyvinyl chloride, polystyrene, polypropylene, PVDF, and nylon.

The NDRG3 protein activity inhibitor preferably inhibits a binding degree of NDRG3 to one or more selected from PKC-β, RACK1 and c-Raf.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

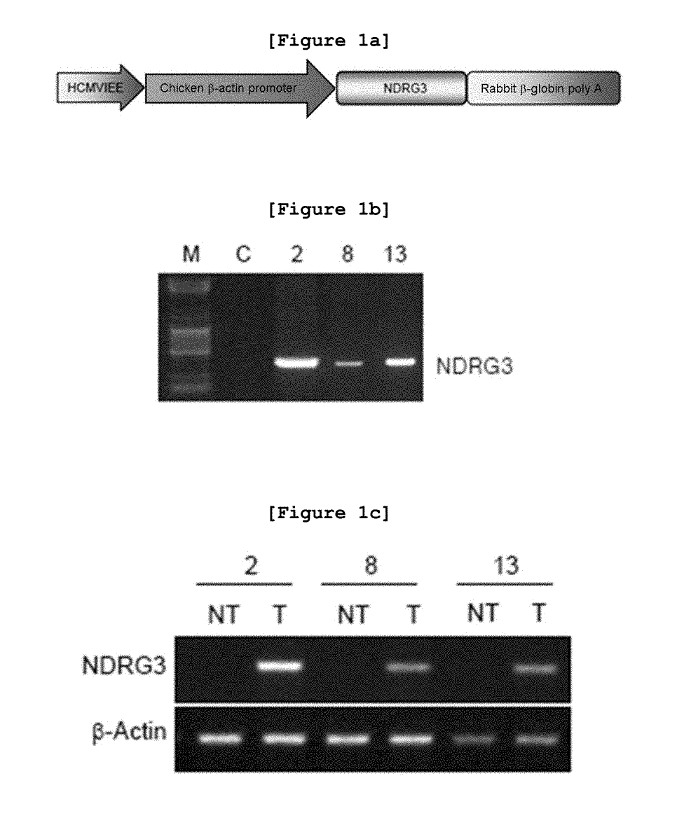

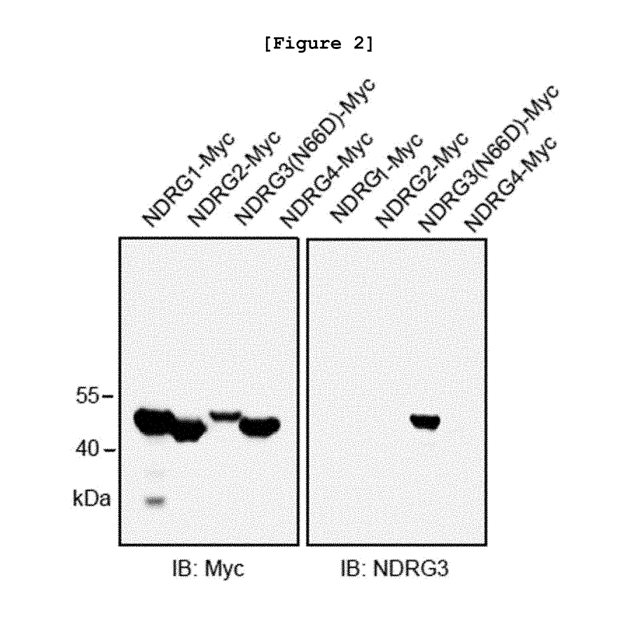

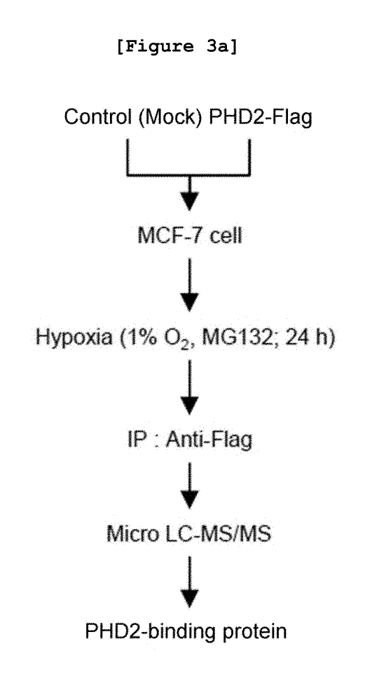

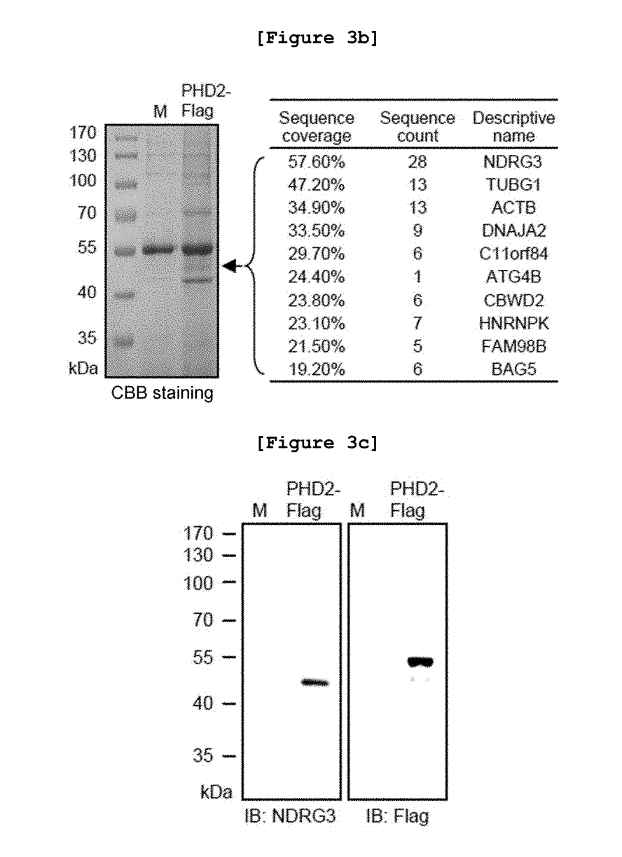

In one specific embodiment of the present invention, the present inventors have selected NDRG3 (SEQ ID NO: 1) from candidate proteins that bind to a PHD2 protein involved in the activity of HIF so as to find an HIF-independent factor associated with hypoxia, preparing NDRG3-overexpressing transgenic C57/BL6 mice TG-2, TG-8 and TG-13 to check the molecular biochemical functions of the NDRG3 protein in the hypoxia (see FIGS. 1A to 1C, and FIGS. 3A to 3C), obtaining anti-NDRG3 polyclonal antisera in a rabbit using a recombinant human NDRG3 protein (consisting of 32^{nd} to 315^{th} amino acids; SEQ ID NO: 2) as an antigen, and purifying an NDRG3 peptide (consisting of 244^{th} to 255^{th} amino acids; SEQ ID NO: 3) by affinity chromatography to prepare a rabbit anti-human NDRG3 polyclonal antibody (see FIG. 2).

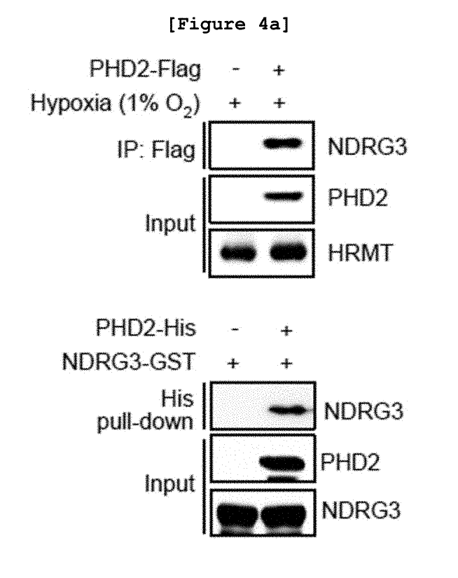

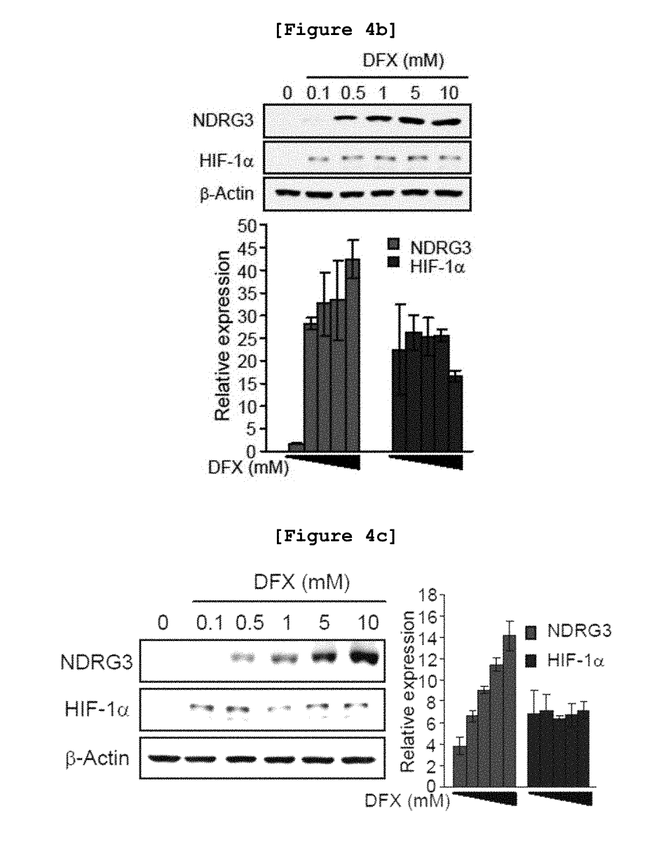

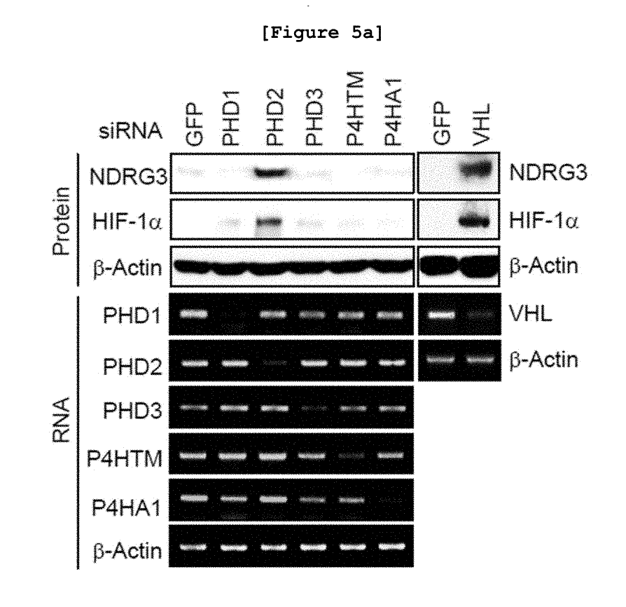

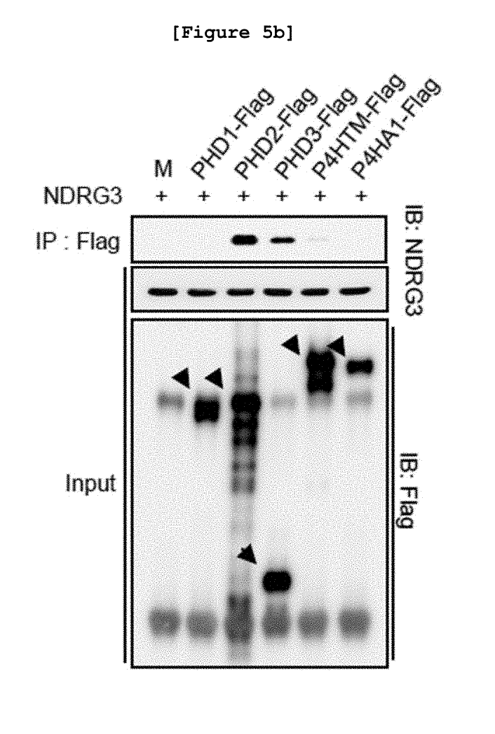

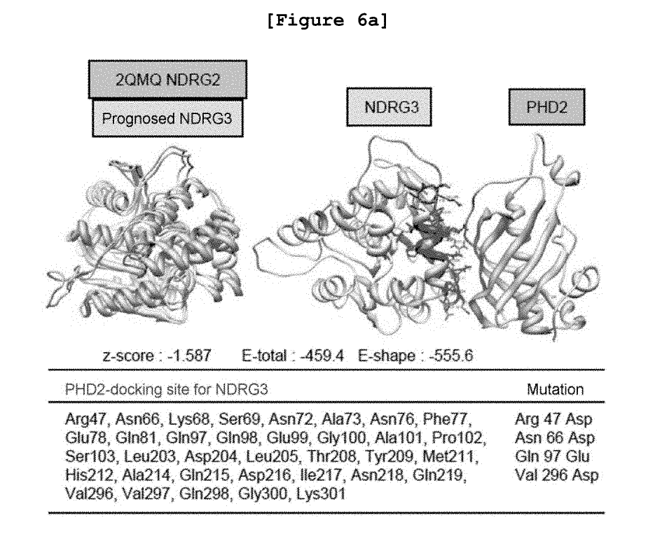

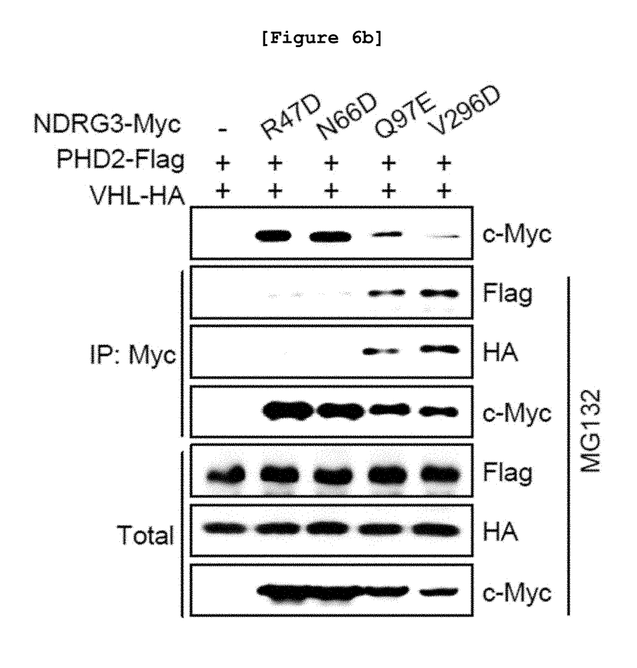

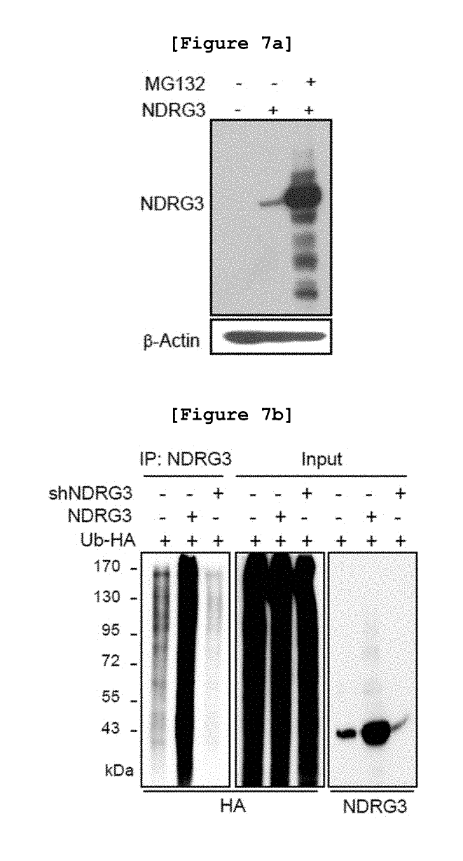

Also, in one specific embodiment of the present invention, the present inventors have conducted an immunoprecipitation assay, Western blotting, an in vitro pull-down assay, and an RT-PCR to verify the relationship between the PHD2 and the NDRG3 protein under oxygen conditions, and verified that the NDRG3 protein binds to a PHD2 protein among four types of PHD family groups and is increasingly accumulated by inhibition of VHL that is a target protein for a PHD2/E3 ubiquitin ligase complex. As a result, the present inventors have found that the NDRG3 protein is an intrinsic substrate of PHD2, and is a substrate serving as NDRG3 in a PHD2/VHL-mediated posttranslational process (see FIGS. 4A to 4C, and FIGS. 5A and 5B). Also, the present inventors have conducted a protein-protein docking simulation to determine PHD2 docking sites of NDRG3, and verified that an amino acid residue at position 47 or 66 of NDRG3 is more important among the PHD2 docking sites (FIGS. 6A and 6B). Also, the present inventors have conducted an in vivo ubiquitination assay, and found that PHD2 binds to the NDRG3 docking sites under a normoxic condition so that NDRG3 is ubiquitinated and removed through a PHD2/VHL-mediated proteasome pathway (see FIGS. 7A and 7B).

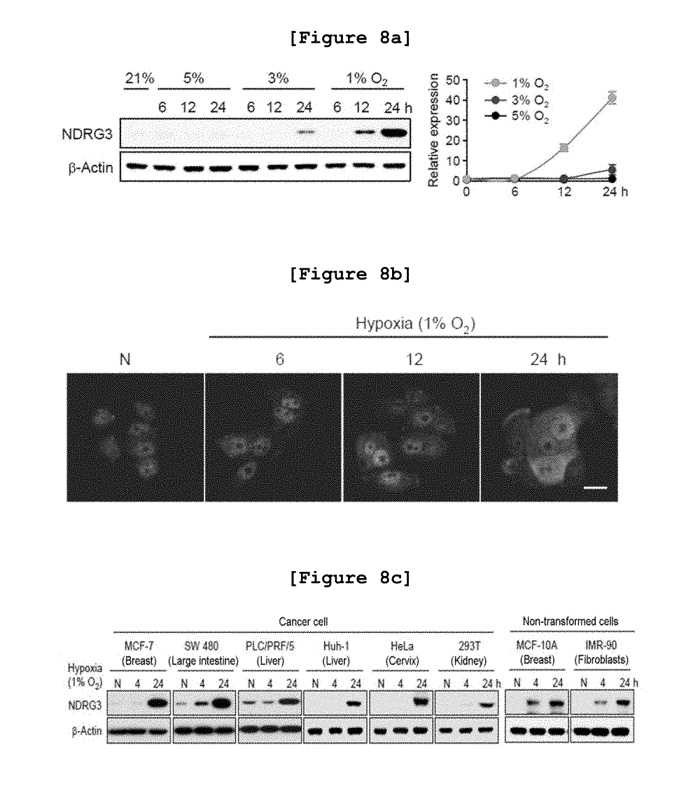

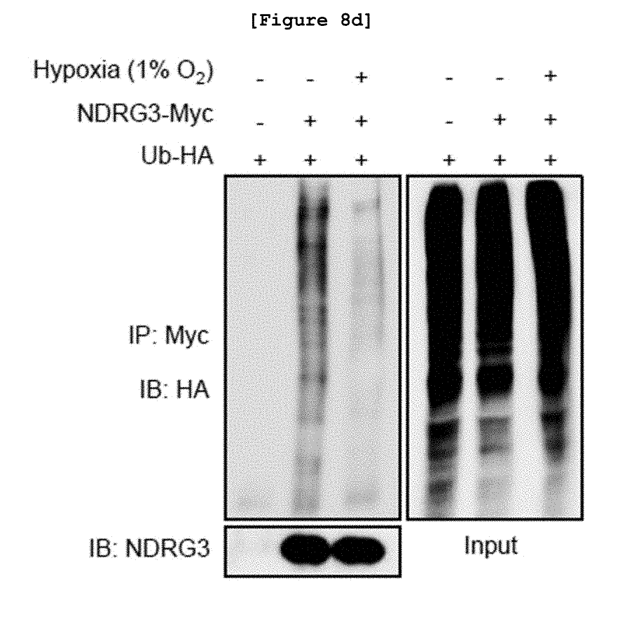

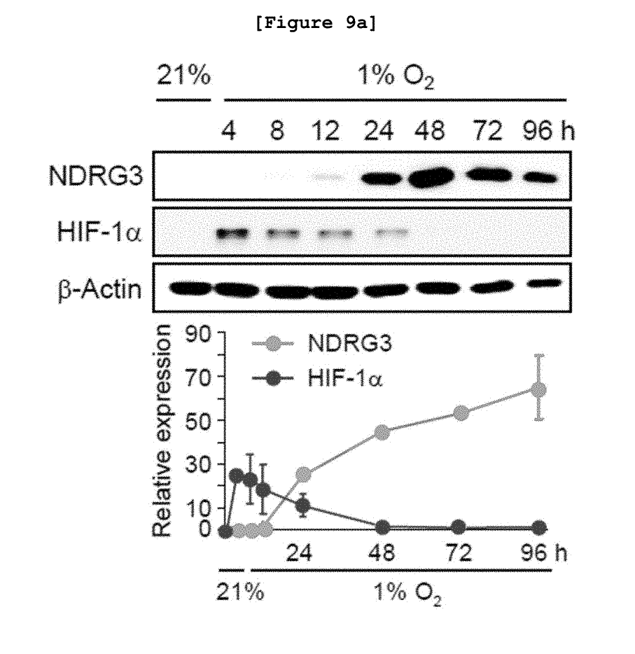

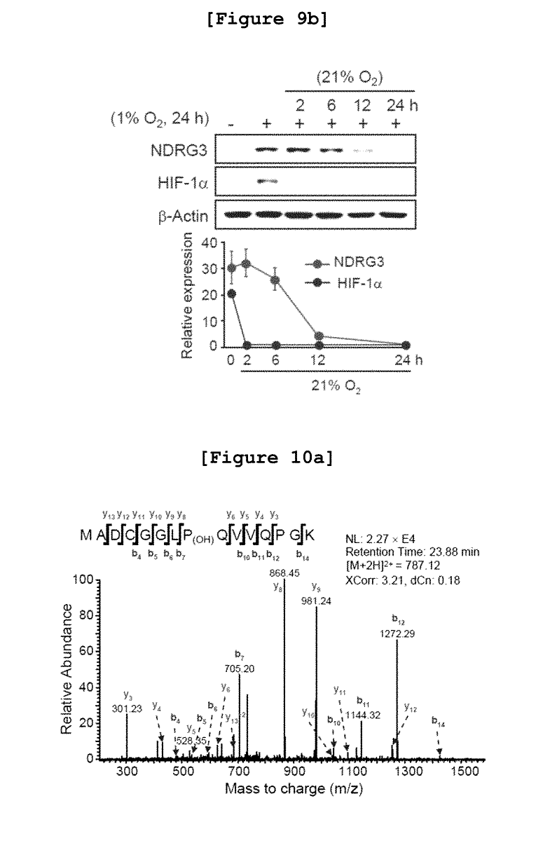

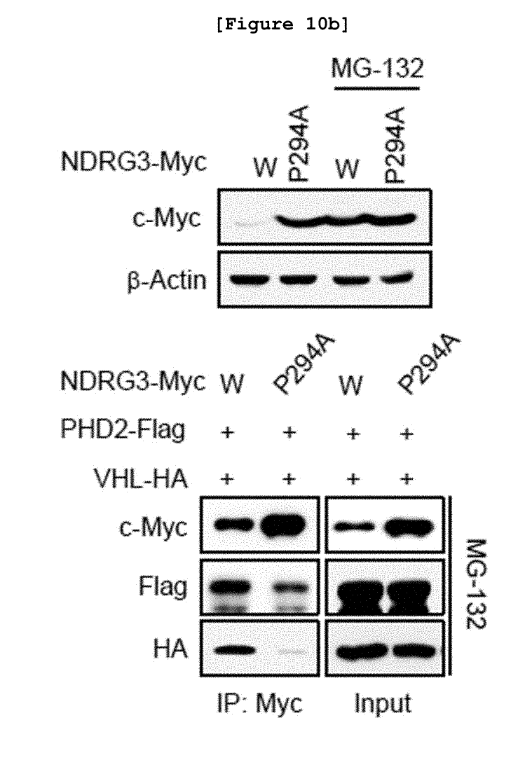

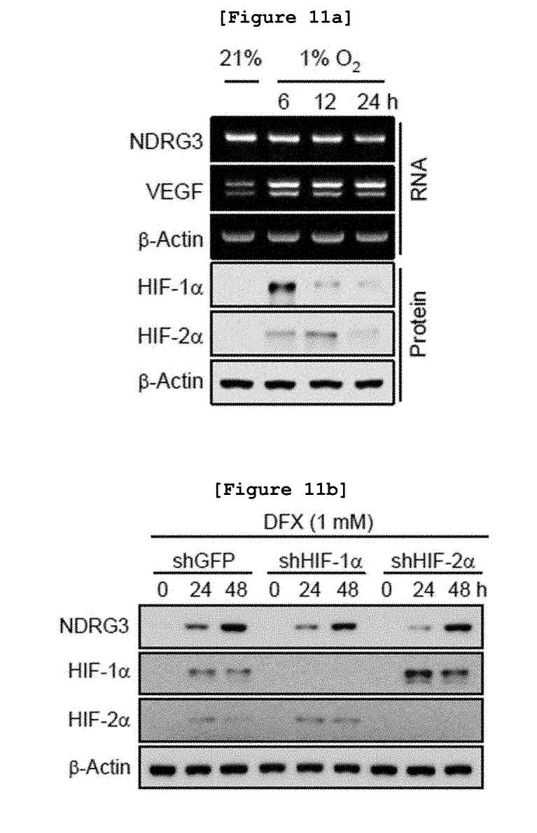

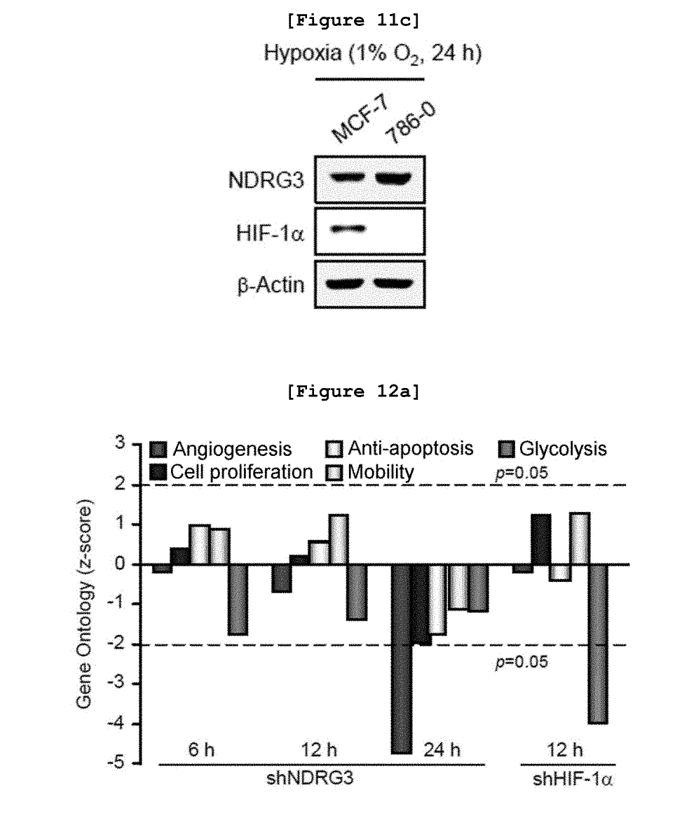

In addition, in one specific embodiment of the present invention, the present inventors have conducted an immunoprecipitation assay, Western blotting, an immunofluorescent staining assay, and an in vivo ubiquitination assay to check oxygen dependence of the NDRG3 protein, and verified that the ubiquitination of the NDRG3 protein decreases and the expression and accumulation of the NDRG3 protein increase as the hypoxic condition lasts various cancer cells such as breast, liver, cervix, kidney, and large intestine, whereas the expression of the NDRG3 protein is slowly eliminated when the hypoxic condition returns to the normoxic condition (see FIGS. 8A to 8D, and FIGS. 9A and 9B), particularly have conducted mass spectrometry, and found that 294^{th} proline of the NDRG3 is a target site that is hydroxylated in an oxygen-dependent manner to eliminate the expression of the NDRG3 protein (FIG. 10A and FIG. 10B). Also, the present inventors have verified that the expression of HIF increases at the beginning of hypoxic condition, and then gradually decreases, but the expression and accumulation of the NDRG3 protein increase as the hypoxic condition last. As a result, the present inventors have found that NDRG3 play an important role in a sustained hypoxic response in an HIF-independent manner (see FIGS. 11A to 11C).

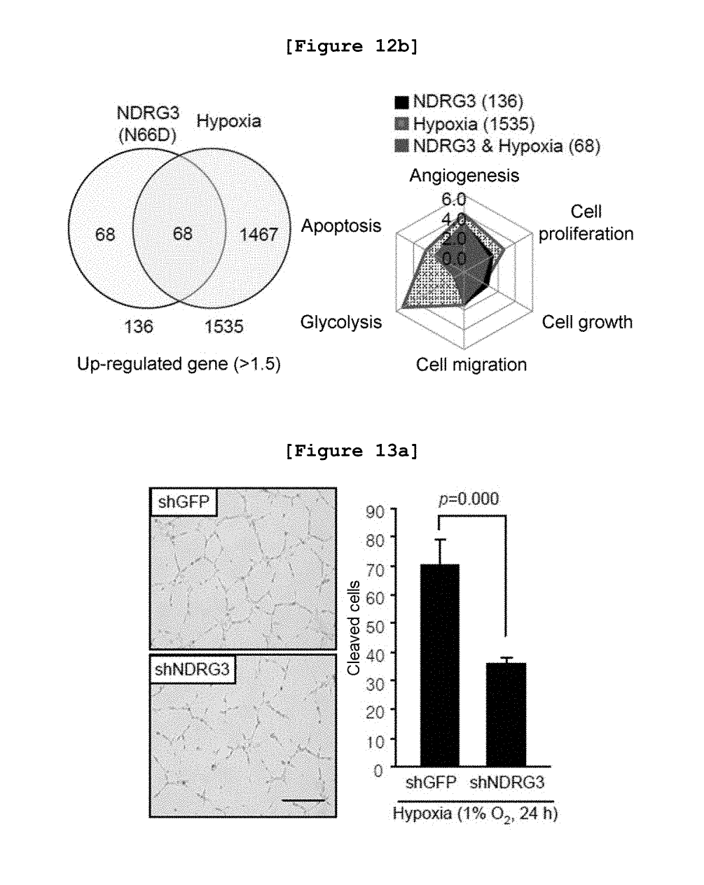

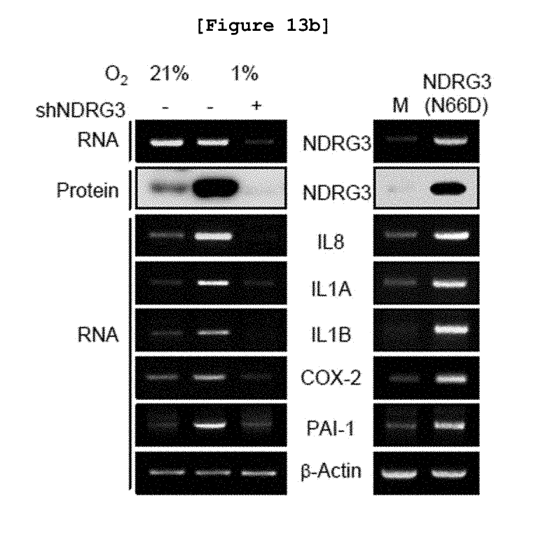

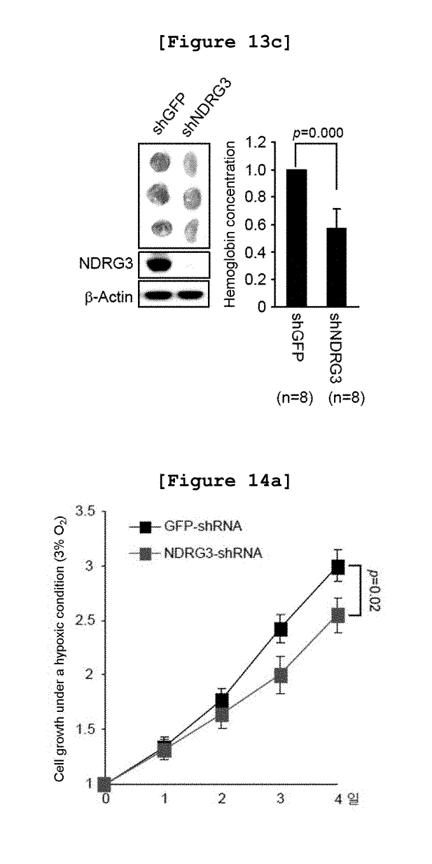

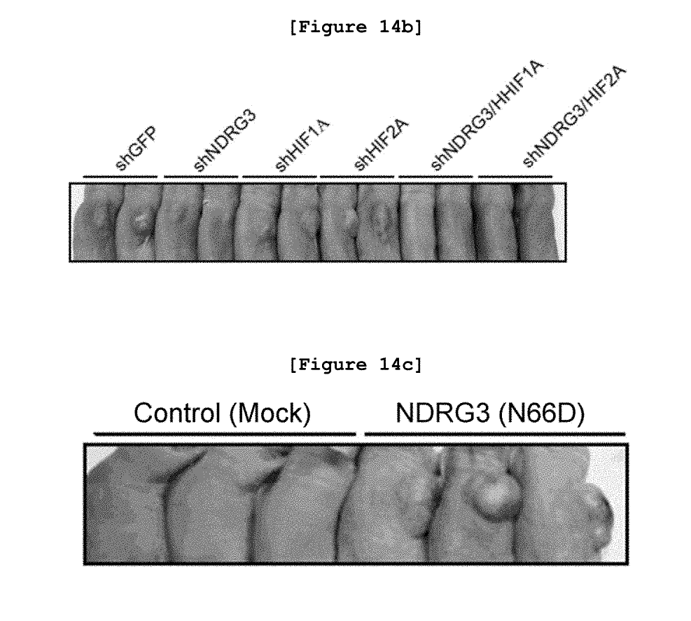

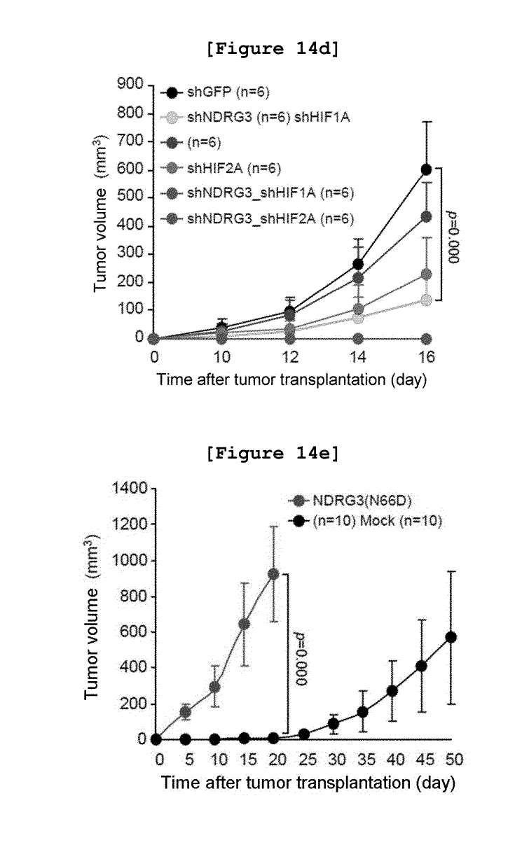

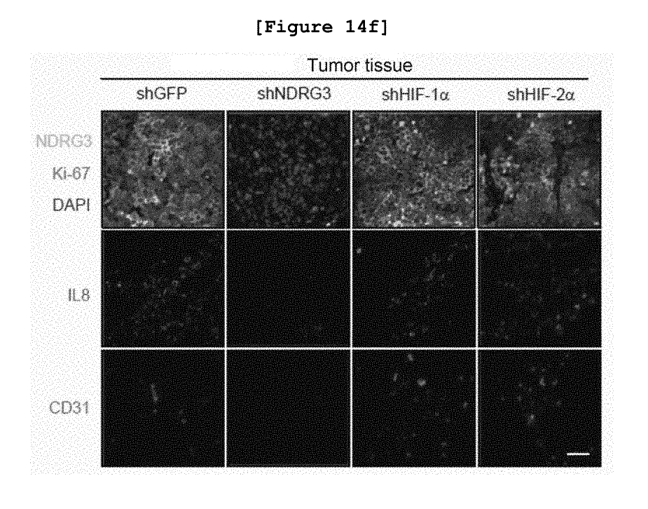

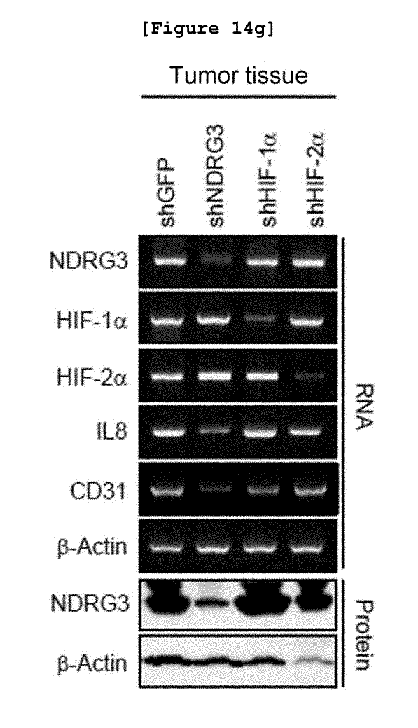

Additionally, in one specific embodiment of the present invention, the present inventors have conducted gene expression profiling to check the biochemical functions of the NDRG3 protein in a hypoxic response, and verified that, unlike HIF, the NDRG3 protein is most highly involved in cell proliferation, angiogenesis, and cell growth (FIGS. 12A and 12B), and also have conducted an in vivo angiogenesis assay, in vivo xenograft tumor volumetry and an MTT assay, and found that the angiogenesis, cell proliferation and tumor growth are promoted by NDRG3 in a hypoxic response (FIGS. 13A and 13C, and FIGS. 14A to 14G).

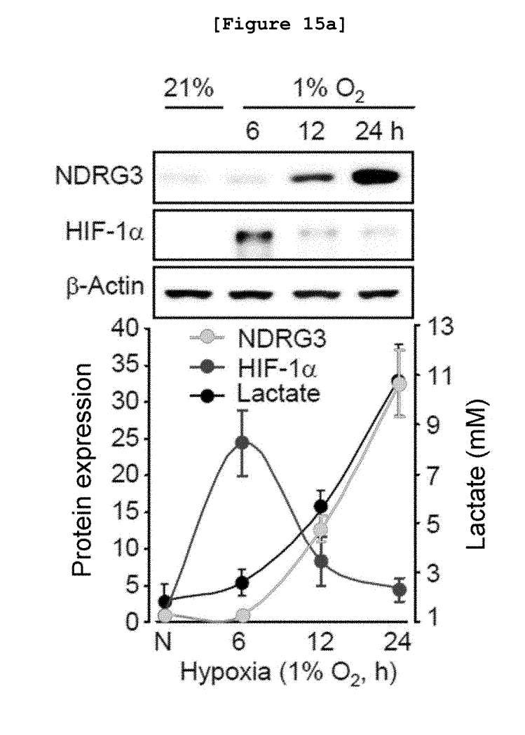

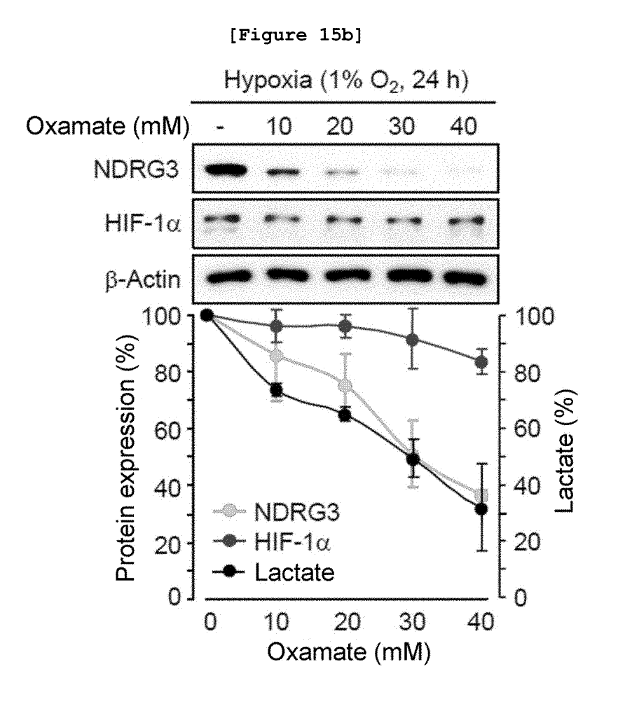

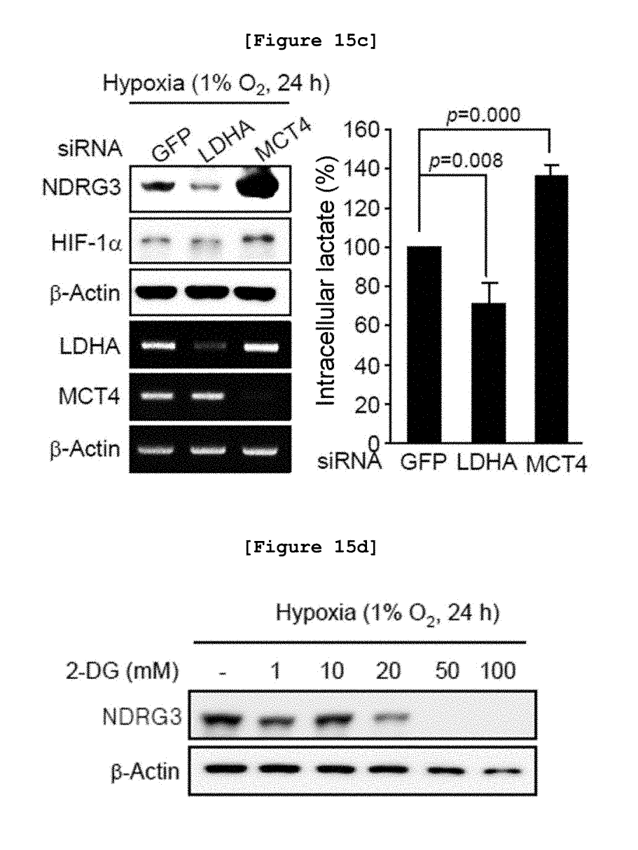

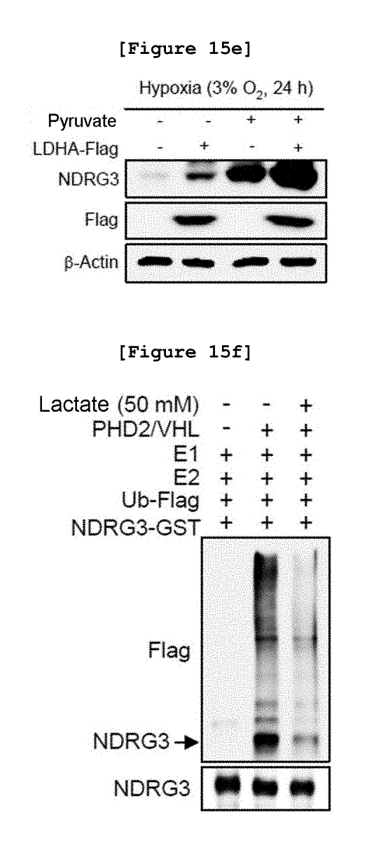

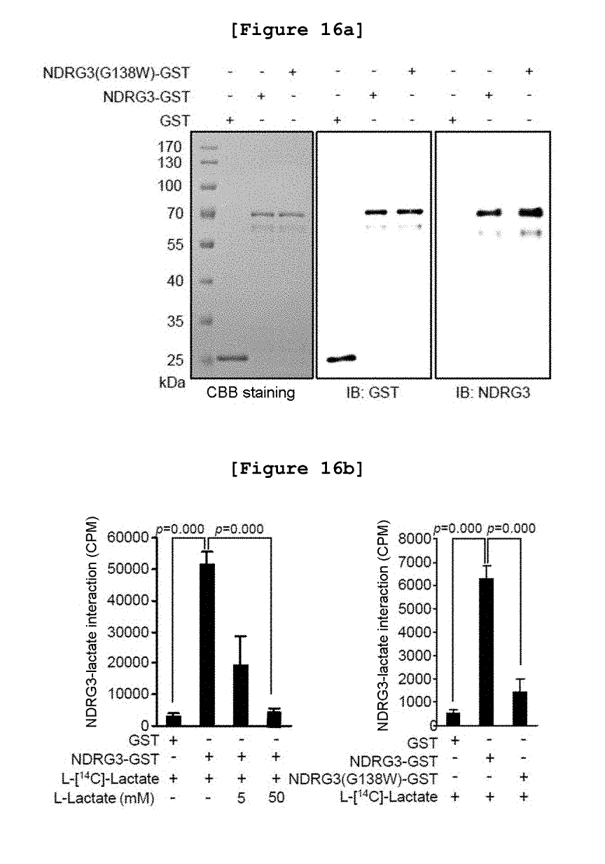

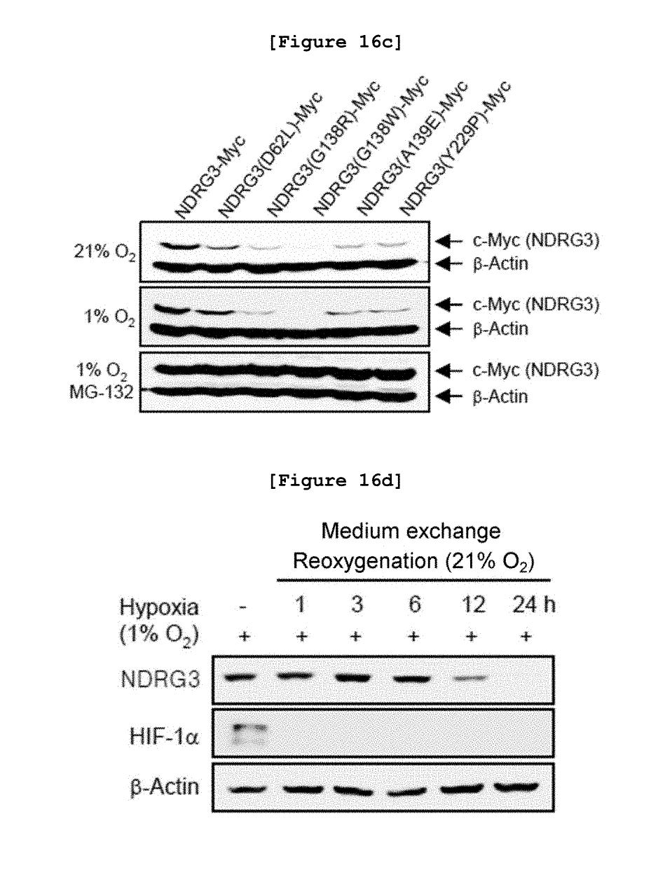

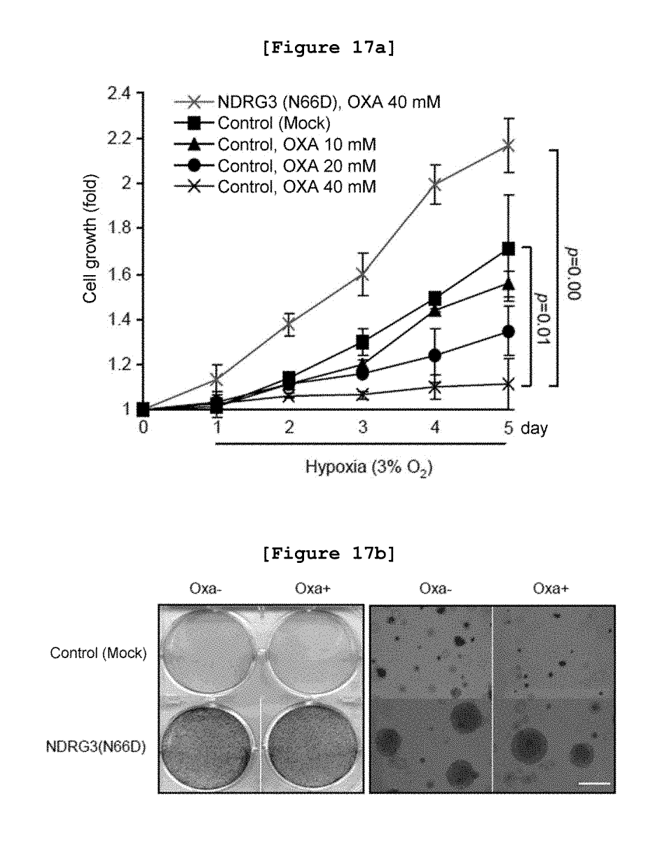

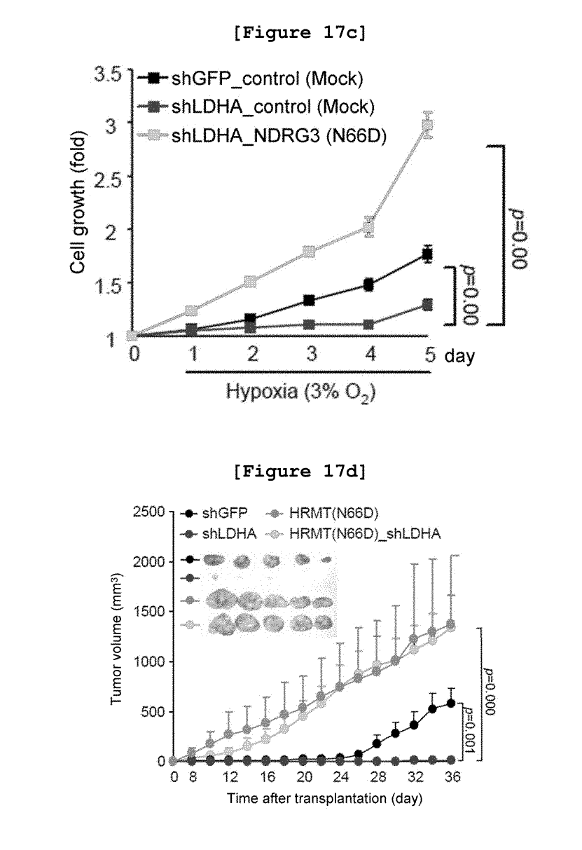

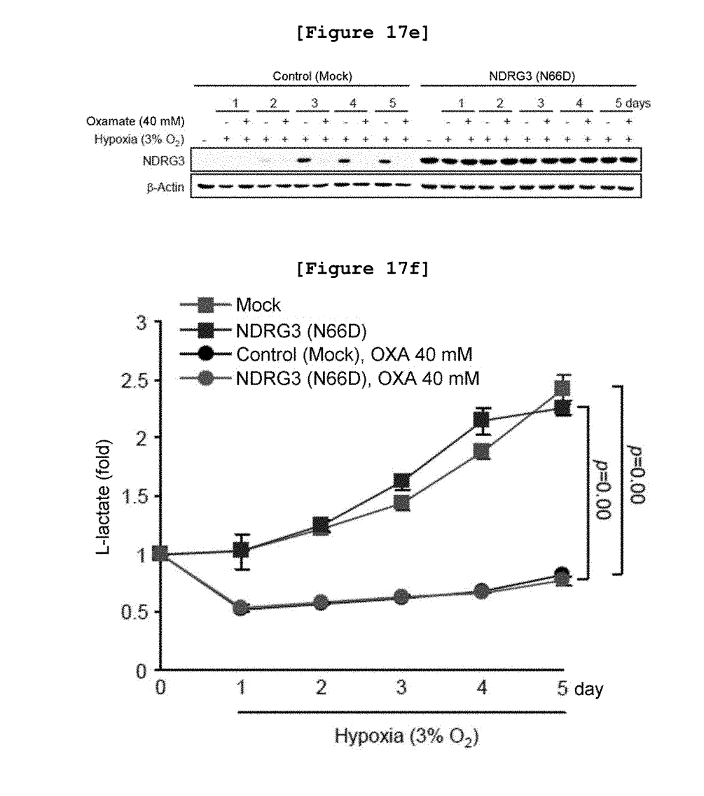

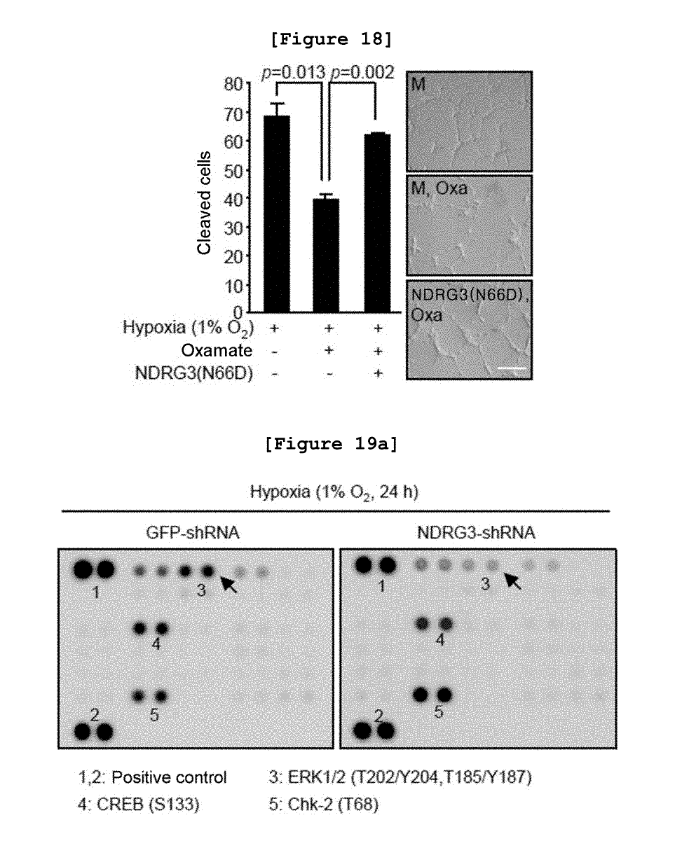

Also, in one specific embodiment of the present invention, the present inventors have determined lactate generation under a hypoxic condition and conducted Western blotting, an immunoprecipitation assay, and an in vitro ubiquitination assay to verify the relationship between the lactate generation under the hypoxic condition and an increase in expression of the NDRG3 protein, and found that lactate is generated from a hypoxic response, and the generated lactate binds to a lactate binding site including a 62^{nd}, 138^{th}, 139^{th} or 229^{th} residue of the NDRG3 to inhibit ubiquitination of the NDRG3 protein and increase expression of the NDRG3 protein in an HIF-independent manner (see FIGS. 15A to 15F, and FIGS. 16A to 16D), and also have conducted an MTT assay, a colony forming assay, in vivo xenograft tumor volumetry, and a tube forming assay, and found that cell proliferation and angiogenesis are promoted even through inhibition of lactate generation when an ectopic variant NDRG3 (N66D) is expressed. Therefore, the present inventors have found that the NDRG3 serves an important mediator for cell proliferation and angiogenesis induced by lactate under the sustained hypoxic condition (see FIGS. 17A to 17F, and FIG. 18).

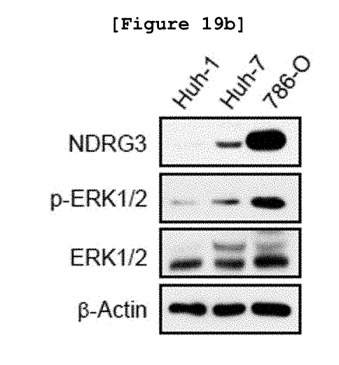

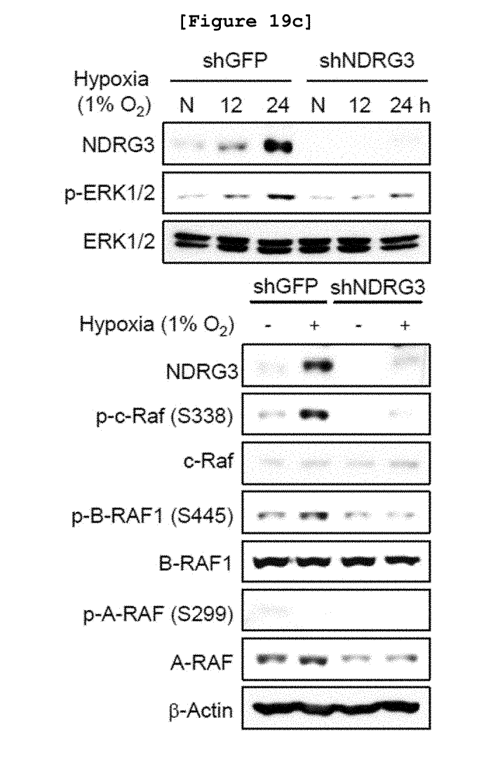

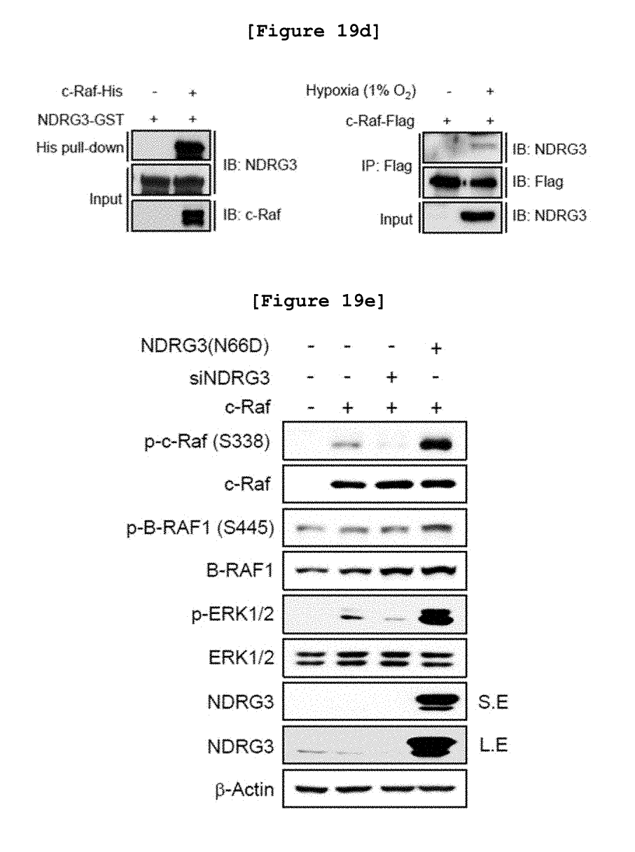

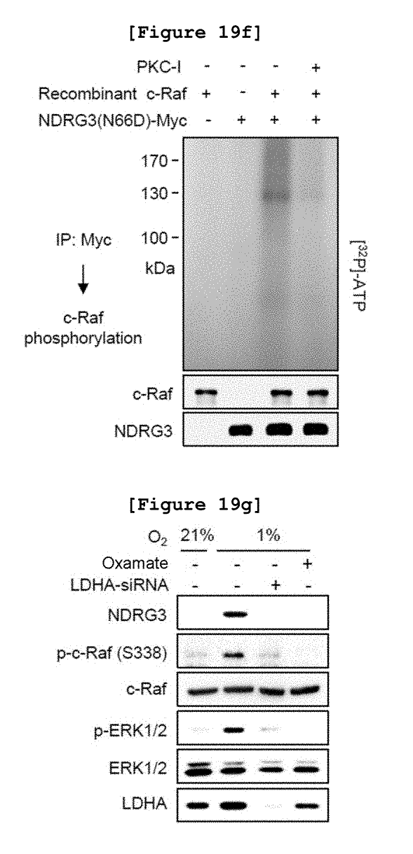

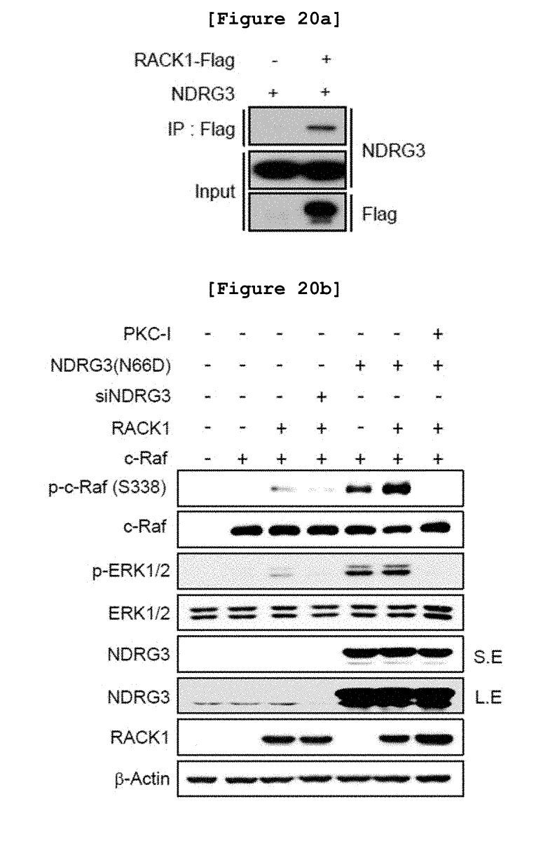

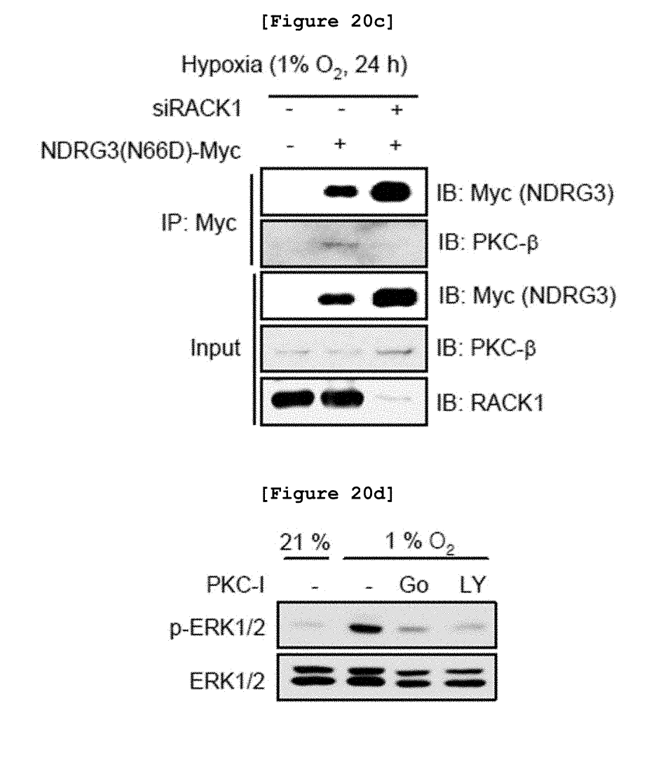

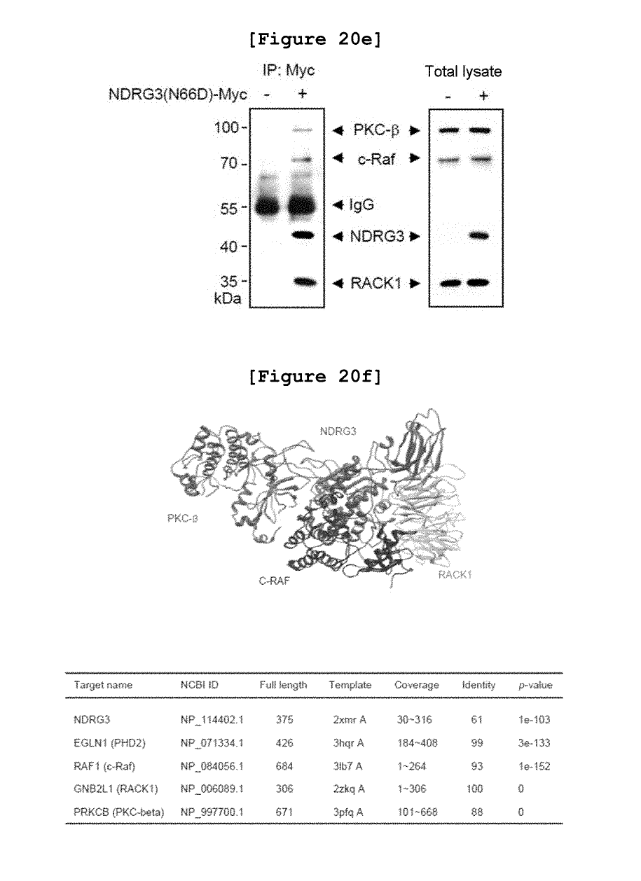

In addition, in one specific embodiment of the present invention, the present inventors have conducted an in vitro kinase assay, a pull-down assay, an immunoprecipitation assay, and Western blotting to check a molecular regulatory mechanism of a hypoxic response mediated by the NDRG3, and verified that phosphorylation of c-Raf and ERK1/2 is inhibited through inhibition of NDRG3 expression under a hypoxic condition. As a result, the present inventors have found that the NDRG3 is an important mediator for c-Raf-ERK1/2 signaling in a c-Raf-ERK1/2 pathway activated by lactate generated from a hypoxic response (see FIGS. 19A to 19G). Also, the present inventors have conducted protein structure modeling, and verified that NDRG3 binds to RACK1 to induce PKC-β and form an NDRG3-RACK1-PKC-β-c-Raf complex with c-Raf, and a c-Raf protein is phosphorylated by the induced PKC-β to activate c-Raf/ERK signaling. As a result, the present inventors have found that the NDRG3 regulated by lactate is a scaffold protein for regulating the activity of c-Raf (see FIGS. 20A to 20F).

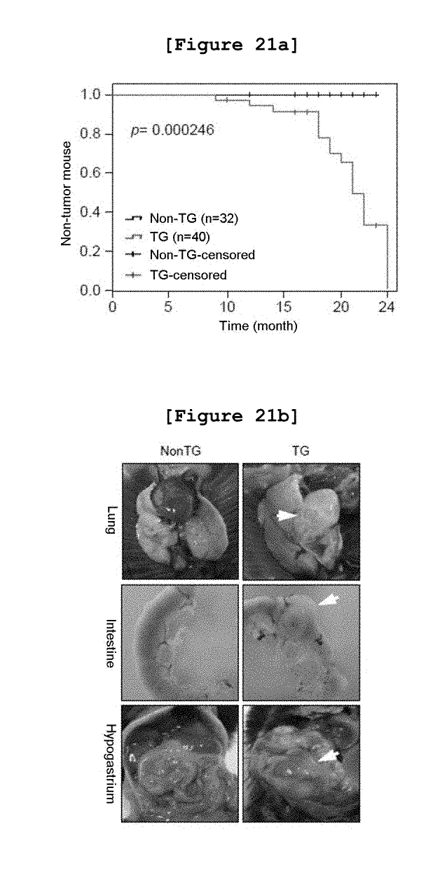

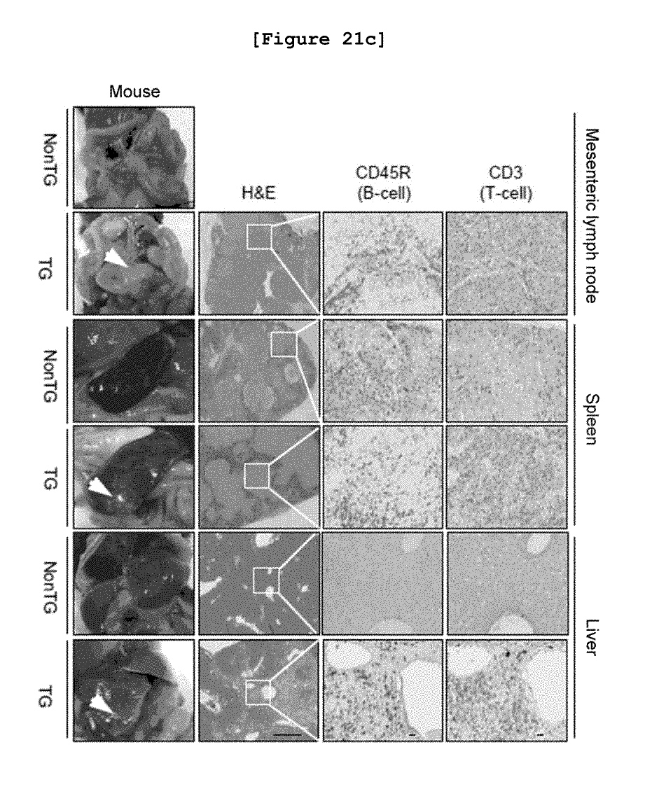

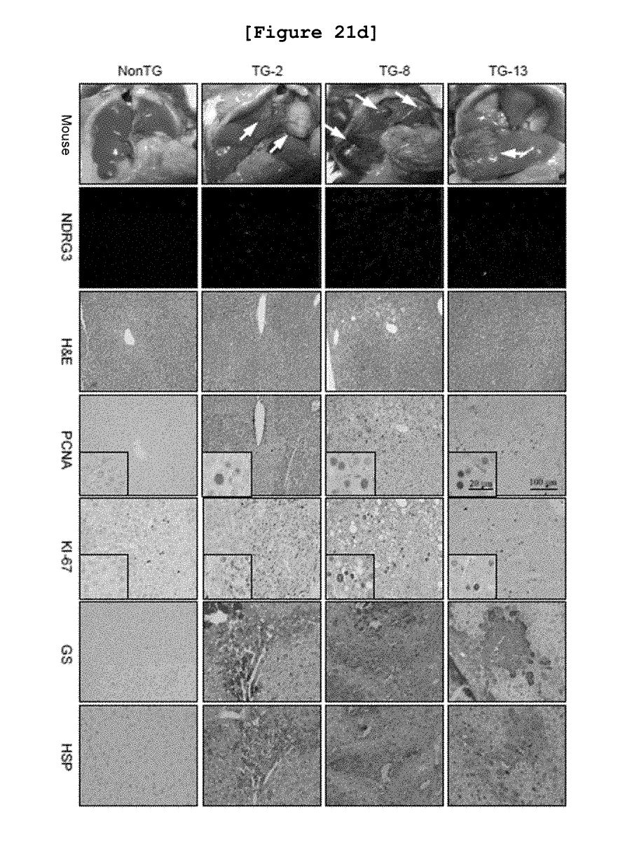

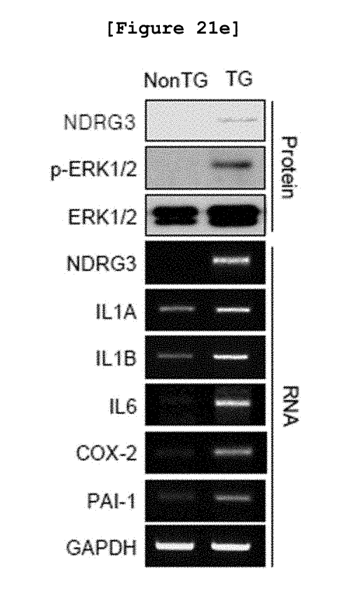

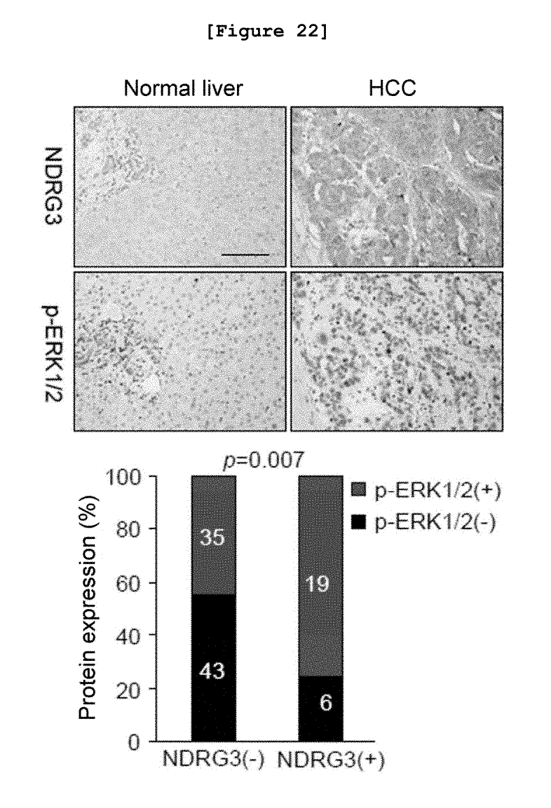

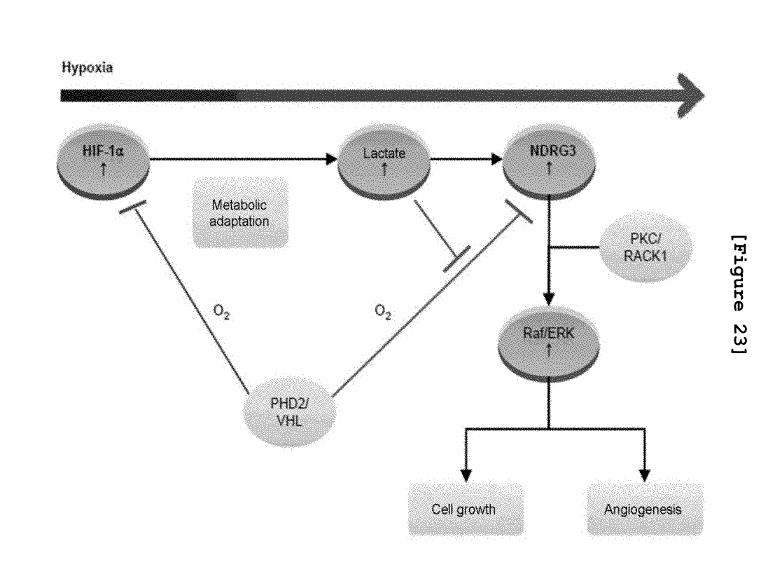

Further, one specific embodiment of the present invention, the present inventors have conducted immunohistochemical analysis using the prepared NDRG3-overexpressing transgenic mice to determine pathologic changes by NDRG3, conducted Western blotting to determine expression of the NDRG3 and activated ERK1/2 protein, and conducted RT-PCR to determine changes in expression of genes, and then verified that tumor is detected in various organs such as lungs, intestines, livers, and the like from the NDRG3-overexpressing transgenic mice, and lymphoma-expressing B cells and T cells are detected in secondary lymphoid organs such as mesenteric lymph node and spleen. As a result, the present inventors have found that cell proliferation markers and angiogenic markers are increasingly expressed (see FIGS. 21A to 21E). Also, the present inventors have verified that the expression of the NDRG3 protein and the activity of the ERK1/2 protein are promoted even in liver cancer tissue samples isolated from a liver cancer patient (see FIG. 22), and found that abnormal expression of NDRG3 activates the c-Raf/ERK pathway to promote tumorigenesis and angiogenesis.

Therefore, in the NDRG3 of the present invention, PHD2 binds to a PHD2 docking site of the NDRG3 under a normoxic condition to be ubiquitinated and down-regulated through ubiquitination by a PHD/VHL-mediated pathway, and accumulation of an HIF-1α protein is induced due to inactivity of PHD2 at the beginning of hypoxic condition so that genes (LDHA, PDK1, etc.) involved in metabolic adaptation of cells in response to hypoxia are up-regulated to activate glycolysis. Thereafter, the NDRG3 protein is increasingly expressed by lactate generated/accumulated by the increased glycolysis as well as inhibition of hydroxylation of 294^{th} proline that is a hypoxic target site of NDRG3 due to the PHD2 inactivity under a hypoxic condition. The increasingly expressed NDRG3 serves as a scaffold protein in a sustained hypoxic response to bind to c-Raf and RACK1, and the bound RACK1 recruits PKC-β proteins to form a complex. The c-Raf is then phosphorylated by PKC to activate a c-Raf-ERK1/2 pathway, thereby promoting cell proliferation and angiogenesis (see FIG. 23). Accordingly, an inhibitor for inhibiting the expression or activity of the NDRG3 may be usefully used as a pharmaceutical composition for preventing and treating a cancer.

The composition of the present invention preferably includes the active ingredient at 0.0001 to 50% by weight, based on the total weight of the composition, but the present invention is not limited thereto.

The composition of the present invention may further include one or more active ingredients that have the same or similar functions. For administration, the composition may be prepared to further include one or more pharmaceutically acceptable carriers. The composition of the present invention includes the protein at 0.0001 to 10% by weight, preferably at 0.001 to 1% by weight, based on the total weight of the composition. The pharmaceutically acceptable carrier may be used in combination with saline, sterile water, a Ringer's solution, buffered saline, a dextrose solution, a maltodextrin solution, glycerol, ethanol, and a combination of one or more components. As necessary, other conventional additives such as antioxidants, buffers, bacteriostatic agents, etc. may be added. Also, a diluent, a dispersant, a surfactant, a binder, and a lubricant may be further added to be prepared into an injectable formulation such as an aqueous solution, a suspension, an emulsion, etc., a pill, a capsule, a granule, or a tablet. Further, the composition may be preferably prepared into preparations in response to respective diseases or components using suitable methods known in the related art, or methods disclosed in the Remington's document (Remington's Pharmaceutical Science (new version), Mack Publishing Company, Easton Pa.).

The composition of the present invention may be parenterally administered (for example, intravenously, intramuscularly, intraperitoneally, subcutaneously or locally applied) using desired methods, and the dosage of the composition may be widely adjusted depending on the weight, age, sex and health condition of a patient, diet, an administration time, a route of administration, an excretion rate, and sensitivity to a disease. Assuming that an adult male weigh 60 kg (based on the U.S. FDA guidance), the composition according to the present invention may be administered at a dose of 0.738 μg to 7.38 g, preferably 7.38 μg to 0.738 g (12.3 mpk), and is preferably administered every other day, but the route of administration may be determined on the patient's demand.

Known therapeutic agents may be directly or indirectly bound to the composition of the present invention, or included in the composition. A therapeutic agent capable of binding to antibodies includes a radionuclide, a drug, a lymphokine, a toxin, and a bispecific antibody. For example, any known therapeutic agents that may bind antibodies or have a therapeutic effect when administered with an antibody, siRNA, shRNA and an antisense oligonucleotide may be all used as the therapeutic agent included in the composition of the present invention, but the present invention is not limited thereto.

The radionuclide may include ³H, ¹¹C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re, but the present invention is not limited thereto.

The drug and toxin may include etoposide, teniposide, adriamycin, daunomycin, carminomycin, aminopterin, dactinomycin, mitomycins, cis-platinum and cis-platinum homologues, bleomycins, esperamicins, 5-fluorouracil, melphalan, and other nitrogen mustards, but the present invention is not limited thereto.

Also, the present invention provides a pharmaceutical composition for preventing and treating a cancer comprising an NDRG3 protein expression or activity inhibitor and a hypoxia-inducible factor (HIF) inhibitor as an active ingredient.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

According to one exemplary embodiment of the present invention, the present inventors have conducted in vivo xenograft tumor volumetry, and verified that tumorigenesis is significantly inhibited when NDRG3 and HIF-1α or HIF-2α are inhibited together. As a result, the present inventors have found that an effect of inhibiting cancer may be improved by inhibiting NDRG3 and HIF at the same time (see FIGS. 14B and 14D).

Therefore, according to the present invention, it is confirmed that glycolysis is activated by a HIF-1α protein at the beginning of hypoxic condition, and the NDRG3 mediated by lactate generated/accumulated from the activation of glycolysis promotes cell proliferation and angiogenesis through a lactate-NDRG3-c-Raf-ERK signaling pathway. Accordingly, a composition including an inhibitor and HIF inhibitor for inhibiting the expression or activity of the NDRG3 may be usefully used for prevention and treatment of a cancer.

Also, the present invention provides a method of detecting an NDRG3 protein to provide information on cancer comprising:

1) measuring expression or activity of the NDRG3 protein from a specimen isolated from a test object; and

2) determining to have cancer or be at risk of having cancer when the expression or activity of the NDRG3 protein in step 1 is increased compared to the normal control.

The specimen in step 1 preferably includes at least one selected from the group consisting of cells, tissues, blood, sera, saliva, and urine, but the present invention is not limited thereto.

A level of expression or activity of the NDRG3 protein in step 1 is preferably measured using at least one selected from the group consisting of an enzyme-linked immunosorbent assay (ELISA), immunohistochemical staining, Western blotting, and protein chips, but the present invention is not limited thereto.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

According to the present invention, since the NDRG3 mediated by lactate generated from a hypoxic response promotes cell proliferation and angiogenesis through a lactate-NDRG3-c-Raf-ERK signaling pathway, the NDRG3 may be usefully used for the method of detecting a protein to provide information on cancer.

In addition, the present invention provides a method of screening a pharmaceutical composition for preventing and treating a cancer comprising:

1) treating an NDRG3 protein-expressing cell line with a test material;

2) confirming expression or activity of an NDRG3 protein in the cell line of step 1; and

3) selecting the test material that reduces the expression or activity of the NDRG3 protein in step 2, compared to the untreated control.

Additionally, the present invention provides a method of screening a pharmaceutical composition for preventing and treating a cancer comprising:

1) treating a cell line, which expresses NDRG3 and one or more selected from PKC-β, RACK1 and c-Raf, with a test material under a hypoxic condition;

2) determining a binding degree of the NDRG3 to one or more selected from the PKC-β, RACK1 and c-Raf in the cell line of step 1; and

3) selecting the test material that reduces the binding degree in step 2, compared to the untreated control.

Also, the present invention provides a method of screening a pharmaceutical composition for preventing and treating a cancer comprising:

1) treating NDRG3, PKC-β, RACK1 and c-Raf proteins with a test material in vitro;

2) determining a binding degree of one or more selected from the NDRG3, PKC-β, RACK1 and c-Raf proteins in step 1; and

3) selecting the test material which reduces the binding degree in step 2, compared to the untreated control.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

According to the present invention, since the NDRG3 mediated by lactate generated from a hypoxic response promotes cell proliferation and angiogenesis through a lactate-NDRG3-c-Raf-ERK signaling pathway, the NDRG3 may be usefully used for the method of screening a pharmaceutical composition for preventing and treating a cancer.

Also, the present invention provides a method of preventing a cancer comprising administering a pharmaceutically effective amount of the NDRG3 protein expression or activity inhibitor to a subject.

In addition, the present invention provides a method of treating a cancer comprising administering a pharmaceutically effective amount of the NDRG3 protein expression or activity inhibitor to a subject.

Additionally, the present invention provides a NDRG3 protein expression or activity inhibitor for use as a pharmaceutical composition for preventing and treating a cancer.

The NDRG3 protein preferably consists of an amino acid sequence set forth in SEQ ID NO: 1.

The NDRG3 protein expression inhibitor preferably includes at least one selected from the group consisting of antisense nucleotide, short interfering RNA, and short hairpin RNA, all of which complementarily bind to mRNA of an NDRG3 gene, but the present invention is not limited thereto.

The NDRG3 protein expression inhibitor preferably promotes hydroxylation of a proline residue at position 294 of the NDRG3 protein, but the present invention is not limited thereto. According to one exemplary embodiment of the present invention, it is confirmed that accumulation of an NDRG3 variant, in which the 294^{th} proline of NDRG3 is substituted with alanine even under a normoxic condition, increases, and interaction of the NDRG3 variant with PHD2/VHL decreases. As a result, it is revealed that the 294^{th} proline of NDRG3 is a site for inhibiting expression of the NDRG3 protein.

The NDRG3 protein expression inhibitor preferably promotes binding of PHD2 to one or more PHD2 docking sites selected from the group consisting of 47^{th} arginine, 66^{th} asparagine, 68^{th} lysine, 69^{th} serine, 72^{nd} asparagine, 73^{rd} alanine, 76^{th} asparagine, 77^{th} phenylalanine, 78^{th} glutamic acid, 81^{st} glutamine, 97^{th} glutamine, 98^{th} glutamine, 99^{th} glutamic acid, 100^{th} glycine, 101^{st} alanine, 102^{nd} proline, 103^{rd} serine, 203^{rd} leucine, 204^{th} aspartic acid, 205^{th} leucine, 208^{th} threonine, 209^{th} tyrosine, 211^{th} methionine, 212^{th} histidine, 214^{th} alanine, 215^{th} glutamine, 216^{th} aspartic acid, 217^{th} isoleucine, 218^{th} asparagine, 219^{th} glutamine, 296^{th} valine, 297^{th} valine, 298^{th} glutamine, 300^{th} glycine, and 301^{st} lysine of the NDRG3 protein, more preferably promotes binding of PHD2 to one or more PHD2 docking sites selected from the group consisting of 47^{th} arginine, 66^{th} asparagine, 68^{th} lysine, 69^{th} serine, 97^{th} glutamine, and 296^{th} valine of the NDRG3 protein, and most preferably promotes binding of PHD2 to one or more PHD2 docking sites selected from the group consisting of 47^{th} arginine, 66^{th} asparagine, and 68^{th} lysine of the NDRG3 protein, but the present invention is not limited thereto.

The NDRG3 protein expression inhibitor preferably inhibits binding of lactate to 62^{nd} aspartic acid, 138^{th} glycine, 139^{th} alanine, or 229^{th} tyrosine which is a lactate binding site of the NDRG3 protein, but the present invention is not limited thereto.

The NDRG3 protein activity inhibitor is preferably an aptamer or antibody that complementarily binds to the NDRG3 protein, but the present invention is not limited thereto.

The NDRG3 protein activity inhibitor preferably inhibits a binding degree of NDRG3 to one or more selected from PKC-β, RACK1 and c-Raf.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

According to the present invention, since the NDRG3 mediated by lactate generated from a hypoxic response promotes cell proliferation and angiogenesis through a lactate-NDRG3-c-Raf-ERK signaling pathway, the NDRG3 protein expression or activity inhibitor may be usefully used to prevent treat a cancer.

Also, the present invention provides a method of preventing a cancer comprising administering pharmaceutically effective amounts of the NDRG3 protein expression or activity inhibitor and the HIF inhibitor to a subject.

In addition, the present invention provides a method of treating a cancer comprising administering pharmaceutically effective amounts of the NDRG3 protein expression or activity inhibitor and the HIF inhibitor to a subject.

Additionally, the present invention provides a NDRG3 protein expression or activity inhibitor and a HIF inhibitor for use as a pharmaceutical composition for preventing and treating a cancer.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

According to the present invention, it is confirmed that glycolysis is activated by a HIF-1α protein at the beginning of hypoxic condition, and the NDRG3 mediated by lactate generated/accumulated from the activation of glycolysis promotes cell proliferation and angiogenesis through a lactate-NDRG3-c-Raf-ERK signaling pathway. Accordingly, a composition including an inhibitor and HIF inhibitor for inhibiting the expression or activity of the NDRG3 may be usefully used for prevention and treatment of a cancer.

Also, the present invention provides a pharmaceutical composition for preventing and treating an inflammatory disease comprising the NDRG3 protein expression or activity inhibitor as an active ingredient.

The NDRG3 protein preferably consists of an amino acid sequence set forth in SEQ ID NO: 1.

The NDRG3 protein expression inhibitor preferably includes at least one selected from the group consisting of antisense nucleotide, short interfering RNA, and short hairpin RNA, all of which complementarily bind to mRNA of an NDRG3 gene, but the present invention is not limited thereto.

The NDRG3 protein expression inhibitor preferably promotes hydroxylation of a proline residue at position 294 of the NDRG3 protein, but the present invention is not limited thereto. According to one exemplary embodiment of the present invention, it is confirmed that accumulation of an NDRG3 variant, in which the 294^{th} proline of NDRG3 is substituted with alanine even under a normoxic condition, increases, and interaction of the NDRG3 variant with PHD2/VHL decreases. As a result, it is revealed that the 294^{th} proline of NDRG3 is a site for inhibiting expression of the NDRG3 protein.

The NDRG3 protein expression inhibitor preferably promotes binding of PHD2 to one or more PHD2 docking sites selected from the group consisting of 47^{th} arginine, 66^{th} asparagine, 68^{th} lysine, 69^{th} serine, 72^{nd} asparagine, 73^{rd} alanine, 76^{th} asparagine, 77^{th} phenylalanine, 78^{th} glutamic acid, 81^{st} glutamine, 97^{th} glutamine, 98^{th} glutamine, 99^{th} glutamic acid, 100^{th} glycine, 101^{st} alanine, 102^{nd} proline, 103^{rd} serine, 203^{rd} leucine, 204^{th} aspartic acid, 205^{th} leucine, 208^{th} threonine, 209^{th} tyrosine, 211^{th} methionine, 212^{th} histidine, 214^{th} alanine, 215^{th} glutamine, 216^{th} aspartic acid, 217^{th} isoleucine, 218^{th} asparagine, 219^{th} glutamine, 296^{th} valine, 297^{th} valine, 298^{th} glutamine, 300^{th} glycine, and 301^{st} lysine of the NDRG3 protein, more preferably promotes the binding of PHD2 to one or more PHD2 docking sites selected from the group consisting of 47^{th} arginine, 66^{th} asparagine, 68^{th} lysine, 69^{th} serine, 97^{th} glutamine, and 296^{th} valine of the NDRG3 protein, further preferably targets an arginine or asparagine residue at position 47 or 66 which is a PHD2 docking site of the NDRG3 protein, and most preferably binds to the 47^{th} arginine or 66^{th} asparagine residue of the NDRG3 protein to increase interaction with PHD2, thereby inhibiting the expression of the NDRG3 protein, but the present invention is not limited thereto. According to one exemplary embodiment of the present invention, it is confirmed that, when a binding affinity of PHD2 to an NDRG3 (R47D) variant in which the 47^{th} arginine of the NDRG3 is substituted with aspartic acid and an NDRG3 (N66D) variant in which the 66^{th} asparagine of the NDRG3 is substituted with aspartic acid is low, and the NDRG3 (N66D) variant is overexpressed, inflammatory cytokines IL-8, IL-1α, IL-1β, COX-2 and PAI-1 are increasingly expressed. As a result, it is revealed that the 47^{th} arginine or 66^{th} asparagine residue of the NDRG3 protein is an important site for docking of PHD2 under a normoxic condition, and the expression of the NDRG3 protein is up-regulated by PHD2.

The NDRG3 protein expression inhibitor preferably inhibits binding of lactate to 62^{nd} aspartic acid, 138^{th} glycine, 139^{th} alanine, or 229^{th} tyrosine which is a lactate binding site of the NDRG3 protein, but the present invention is not limited thereto.

The NDRG3 protein activity inhibitor is preferably an aptamer or antibody that complementarily binds to the NDRG3 protein, but the present invention is not limited thereto.

The NDRG3 protein activity inhibitor preferably inhibits a binding degree of NDRG3 to one or more selected from PKC-β, RACK1 and c-Raf.

The NDRG3 protein expression or activity inhibitor may prevent or treat an inflammatory disease by inhibiting expression or activity of the NDRG3 protein to inhibit the expression of the inflammatory cytokines which are observed at the inflammatory disease, but the present invention is not limited thereto. Also, the inflammatory cytokines include IL-1α, IL-1β, IL-6, IL-8, COX-2, and PAI-1, but the present invention is not limited thereto.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

In the NDRG3 of the present invention, PHD2 binds to the PHD2 docking site of the NDRG3 under a normoxic condition to be down-regulated through ubiquitination by a PHD/VHL-mediated pathway, and accumulation of an HIF-1α protein is induced due to inactivity of PHD2 at the beginning of hypoxic condition so that genes (LDHA, PDK1, etc.) involved in metabolic adaptation of cells in response to hypoxia are up-regulated to activate glycolysis. Thereafter, the NDRG3 protein is increasingly expressed by lactate generated/accumulated by the increased glycolysis as well as inhibition of hydroxylation of 294^{th} proline that is a hypoxic target site of NDRG3 due to the PHD2 inactivity under a hypoxic condition. The increasingly expressed NDRG3 serves as a scaffold protein in a sustained hypoxic response to bind to c-Raf and RACK1, and the bound RACK1 recruits PKC-β proteins to form a complex. The c-Raf is then phosphorylated by PKC to activate a c-Raf-ERK1/2 pathway, thereby promoting the expression of the cytokines that mediate cell proliferation, angiogenesis and inflammatory response (see FIG. 23). Accordingly, an inhibitor for inhibiting the expression or activity of the NDRG3 may be usefully used as a pharmaceutical composition for preventing and treating a cancer or inflammatory disease.

Also, the present invention provides a method of detecting an NDRG3 protein to provide information on diagnosis of an inflammatory disease comprising:

1) measuring expression or activity of the NDRG3 protein from a specimen isolated from a test object; and

2) diagnosing to have the inflammatory disease or predicting to be at risk of having the inflammatory disease when the expression or activity of the NDRG3 protein in step 1 is increased compared to the normal control.

The specimen in step 1 preferably includes at least one selected from the group consisting of cells, tissues, blood, sera, saliva, and urine, but the present invention is not limited thereto.

A level of expression or activity of the NDRG3 protein in step 1 is preferably measured using at least one selected from the group consisting of an enzyme-linked immunosorbent assay (ELISA), immunohistochemical staining, Western blotting, and protein chips, but the present invention is not limited thereto.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, since the NDRG3 mediated by lactate generated from a hypoxic response promotes expression of the cytokines that mediate cell proliferation, angiogenesis and inflammatory response through a lactate-NDRG3-c-Raf-ERK signaling pathway, the NDRG3 may be usefully used for the method of detecting a protein to provide information on diagnosis of an inflammatory disease.

Also, the present invention provides a method of screening a pharmaceutical composition for preventing and treating an inflammatory disease comprising:

1) treating an NDRG3 protein-expressing cell line with a test material;

2) confirming expression or activity of the NDRG3 protein in the cell line of step 1; and

3) selecting the test material which reduces the expression or activity of the NDRG3 protein in step 2, compared to the untreated control.

In addition, the present invention provides a method of screening a pharmaceutical composition for preventing and treating an inflammatory disease comprising:

1) treating a cell line, which expresses NDRG3 and one or more selected from PKC-β, RACK1 and c-Raf, with a test material under a hypoxic condition;

2) determining a binding degree of the NDRG3 to one or more selected from the PKC-β, RACK1 and c-Raf in the cell line of step 1; and

3) selecting the test material which reduces the binding degree in step 2, compared to the untreated control.

Additionally, the present invention provides a method of screening a pharmaceutical composition for preventing and treating an inflammatory disease comprising:

1) treating NDRG3, PKC-β, RACK1 and c-Raf proteins with a test material in vitro;

2) determining a binding degree of one or more selected from the NDRG3, PKC-β, RACK1 and c-Raf proteins in step 1; and

3) selecting the test material which reduces the binding degree in step 2, compared to the untreated control.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, since the NDRG3 mediated by lactate generated from a hypoxic response promotes expression of the cytokines that mediate cell proliferation, angiogenesis and inflammatory response through a lactate-NDRG3-c-Raf-ERK signaling pathway, the NDRG3 may be usefully used for the method of screening a pharmaceutical composition for preventing and treating a cancer or inflammatory disease.

Also, the present invention provides a method of preventing an inflammatory disease comprising administering a pharmaceutically effective amount of the NDRG3 protein expression or activity inhibitor to a subject.

In addition, the present invention provides a method of treating an inflammatory disease comprising administering a pharmaceutically effective amount of the NDRG3 protein expression or activity inhibitor to a subject.

Additionally, the present invention provides a NDRG3 protein expression or activity inhibitor for use as a pharmaceutical composition for preventing and treating an inflammatory disease.

The NDRG3 protein preferably consists of an amino acid sequence set forth in SEQ ID NO: 1.

The NDRG3 protein expression inhibitor preferably includes at least one selected from the group consisting of antisense nucleotide, short interfering RNA, and short hairpin RNA, all of which complementarily bind to mRNA of an NDRG3 gene, but the present invention is not limited thereto.

The NDRG3 protein expression inhibitor preferably promotes hydroxylation of a proline residue at position 294 of the NDRG3 protein, but the present invention is not limited thereto. According to one exemplary embodiment of the present invention, it is confirmed that accumulation of an NDRG3 variant, in which the 294^{th} proline of NDRG3 is substituted with alanine even under a normoxic condition, increases, and interaction of the NDRG3 variant with PHD2/VHL decreases. As a result, it is revealed that the 294^{th} proline of NDRG3 is a site for inhibiting expression of the NDRG3 protein.

The NDRG3 protein expression inhibitor preferably promotes binding of PHD2 to one or more PHD2 docking sites selected from the group consisting of 47^{th} arginine, 66^{th} asparagine, 68^{th} lysine, 69^{th} serine, 72^{nd} asparagine, 73^{rd} alanine, 76^{th} asparagine, 77^{th} phenylalanine, 78^{th} glutamic acid, 81^{st} glutamine, 97^{th} glutamine, 98^{th} glutamine, 99^{th} glutamic acid, 100^{th} glycine, 101^{st} alanine, 102^{nd} proline, 103^{rd} serine, 203^{rd} leucine, 204^{th} aspartic acid, 205^{th} leucine, 208^{th} threonine, 209^{th} tyrosine, 211^{th} methionine, 212^{th} histidine, 214^{th} alanine, 215^{th} glutamine, 216^{th} aspartic acid, 217^{th} isoleucine, 218^{th} asparagine, 219^{th} glutamine, 296^{th} valine, 297^{th} valine, 298^{th} glutamine, 300^{th} glycine, and 301^{st} lysine of the NDRG3 protein, more preferably promotes the binding of PHD2 to one or more PHD2 docking sites selected from the group consisting of 47^{th} arginine, 66^{th} asparagine, 68^{th} lysine, 69^{th} serine, 97^{th} glutamine, and 296^{th} valine of the NDRG3 protein, further preferably targets an arginine or asparagine residue at position 47 or 66 which is a PHD2 docking site of the NDRG3 protein, and most preferably binds to the 47^{th} arginine or 66^{th} asparagine residue of the NDRG3 protein to increase interaction with PHD2, thereby inhibiting the expression of the NDRG3 protein, but the present invention is not limited thereto. According to one exemplary embodiment of the present invention, it is confirmed that, when a binding affinity of PHD2 to an NDRG3 (R47D) variant in which the 47^{th} arginine of the NDRG3 is substituted with aspartic acid and an NDRG3 (N66D) variant in which the 66^{th} asparagine of the NDRG3 is substituted with aspartic acid is low, and the NDRG3 (N66D) variant is overexpressed, inflammatory cytokines IL-8, IL-1α, IL-1β, COX-2 and PAI-1 are increasingly expressed. As a result, it is revealed that the 47^{th} arginine or 66^{th} asparagine residue of the NDRG3 protein is an important site for docking of PHD2 under a normoxic condition, and the expression of the NDRG3 protein is up-regulated by PHD2.

The NDRG3 protein expression inhibitor preferably inhibits binding of lactate to 62^{nd} aspartic acid, 138^{th} glycine, 139^{th} alanine, or 229^{th} tyrosine which is a lactate binding site of the NDRG3 protein, but the present invention is not limited thereto.

The NDRG3 protein activity inhibitor is preferably an aptamer or antibody that complementarily binds to the NDRG3 protein, but the present invention is not limited thereto.

The NDRG3 protein activity inhibitor preferably inhibits a binding degree of the NDRG3 to one or more selected from PKC-β, RACK1 and c-Raf.

The NDRG3 protein expression or activity inhibitor may prevent or treat an inflammatory disease by inhibiting expression or activity of the NDRG3 protein to inhibit the expression of the inflammatory cytokines which are observed at the inflammatory disease, but the present invention is not limited thereto. Also, the inflammatory cytokines include IL-1α, IL-1β, IL-6, IL-8, COX-2, and PAI-1, but the present invention is not limited thereto.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, since the NDRG3 mediated by lactate generated from a hypoxic response promotes expression of the cytokines that mediate cell proliferation, angiogenesis and inflammatory response through a lactate-NDRG3-c-Raf-ERK signaling pathway, an inhibitor for inhibiting the expression or activity of the NDRG3 may be usefully used as the pharmaceutical composition for preventing and treating a cancer or inflammatory disease.

Also, the present invention provides an antibody or an immunologically active fragment thereof which specifically binds to an N-myc downstream-regulated gene 3 (NDRG3) epitope consisting of an amino acid sequence set forth in SEQ ID NO: 3.

The term “antibody” used in the present invention includes a whole form of an antibody and a functional fragment of the antibody molecule. The whole antibody structurally has two full-length light chains and two full-length heavy chains. The light chains are linked with the heavy chains via disulfide bonds. The functional fragment of the antibody molecule refers to a fragment that has an antigen binding function, and examples of the antibody fragment include (i) an Fab fragment consisting of a variable region (VL) of the light chain and a variable region (VH) of the heavy chain, and a constant region (CL) of the light chain and a first constant region (CH1) of the heavy chain; (ii) an Fd fragment consisting of VH and CH1 domains; (iii) an Fv fragment consisting of VL and VH domains from a single antibody; (iv) a dAb fragment consisting of a VH domain (Ward, E. S. et al., Nature 341: 544-546(1989)]; (v) an isolated CDR region; (vi) F(ab′)2 fragment that is a bivalent fragment including two liked Fab fragments; (vii) a single-chain Fv molecule (scFv) linked through a peptide linker to link a VH domain and a VL domain to form an antigen binding site; (viii) a bispecific single-chain Fv dimer; and (ix) a diabody that is a polyvalent or multispecific fragment prepared by gene fusion.

The antibody preferably includes at least one selected from the group consisting of a polyclonal antibody, a monoclonal antibody, a murine antibody, a chimeric antibody, and a humanized antibody, but the present invention is not limited thereto.

The polyclonal antibody may be produced using a conventional method which includes injecting one of protein markers according to the present invention into an animal, and gathering blood from the corresponding animal to obtain sera including the antibody. Such a polyclonal antibody may be purified using any methods known in the related art, and may be prepared from any animal species host such as a goat, a rabbit, a sheep, a monkey, a horse, a pig, cattle, a dog, etc.

The monoclonal antibody may be prepared using any techniques of continuously culturing a cell line to provide production of antibody molecules. Such techniques include a hybridoma technique, a human B-cell hybridoma technique, and an EBV-hybridoma technique, but the present invention is not limited thereto (Kohler G et al., Nature 256: 495-497, 1975; Kozbor D et al., J Immunol Methods 81: 31-42, 1985; Cote R J et al., Proc Natl Acad Sci 80: 2026-2030, 1983; and Cole S P et al., Mol Cell Biol 62:109-120, 1984).

The chimeric antibody includes an antibody consisting of a constant region sequence derived from different species, like an antibody consisting of a variable region sequence derived from one species, a variable region sequence derived from a mouse antibody, and constant regions derived from a human antibody.

The humanized antibody includes an antibody in which a CDR sequence derived from a germ line of another mammal species such as a mouse is conjugated with a human framework region. An additional modification of the framework region may be realized in a human framework sequence as well as in a CDR sequence derived from a germ line of still another mammal species.

The immunologically active fragment preferably includes at least one selected from the group consisting of Fab, Fab′, F(ab′)₂, Fv, Fd, single-chain Fv (scFv), and disulfide-stabilized Fv (dsFv), but the present invention is not limited thereto.

According to one specific embodiment of the present invention, the present inventors have selected NDRG3 (SEQ ID NO: 1) from candidate proteins that bind to a PHD2 protein associated with the activity of HIF so as to find an HIF-independent factor involved in hypoxia, obtaining anti-NDRG3 polyclonal antisera in a rabbit using a recombinant human NDRG3 protein (consisting of 32^{nd} to 315^{th} amino acids; SEQ ID NO: 2) as an antigen, and purifying an NDRG3 peptide (consisting of 244^{th} to 255^{th} amino acids; SEQ ID NO: 3) by affinity chromatography to prepare a rabbit anti-human NDRG3 polyclonal antibody (see FIG. 2).

Also, to check the molecular biochemical functions of the NDRG3 protein in the hypoxia, NDRG3-overexpressing transgenic C57/BL6 mice TG-2, TG-8 and TG-13 are prepared (see FIGS. 1A to 1C, and FIGS. 3A to 3C). The prepared anti-human NDRG3 polyclonal antibody is used to check biological functions in the NDRG3-overexpressing transgenic mice and other various types of cells.

In the NDRG3 of the present invention, PHD2 binds to the PHD2 docking site of the NDRG3 under a normoxic condition to be down-regulated through ubiquitination by a PHD/VHL-mediated pathway, and accumulation of an HIF-1α protein is induced due to inactivity of PHD2 at the beginning of hypoxic condition so that genes (LDHA, PDK1, etc.) involved in metabolic adaptation of cells in response to hypoxia are up-regulated to activate glycolysis. Thereafter, the NDRG3 protein is increasingly expressed by lactate generated/accumulated by the increased glycolysis as well as inhibition of hydroxylation of 294^{th} proline that is a hypoxic target site of NDRG3 due to the PHD2 inactivity under a hypoxic condition. The increasingly expressed NDRG3 serves as a scaffold protein in a sustained hypoxic response to bind to c-Raf and RACK1, and the bound RACK1 recruits PKC-β proteins to form a complex. The c-Raf is then phosphorylated by PKC to activate a c-Raf-ERK1/2 pathway, thereby promoting the expression of the cytokines that mediate cell proliferation, angiogenesis and inflammatory response (see FIG. 23). Accordingly, the antibody or an immunologically active fragment thereof that specifically binds to the NDRG3 epitope may be useful used to conduct research on onset mechanisms of diseases, such as cancers, inflammatory diseases, and the like, caused by hypoxia, screen genes involved in the onset mechanisms, and develop therapeutic agents and new pharmaceuticals.

Also, the present invention provides a composition comprising an antibody or an immunologically active fragment thereof that specifically binds to the NDRG3 epitope consisting of an amino acid sequence set forth in SEQ ID NO: 3.

In addition, the present invention provides a pharmaceutical composition for preventing and treating a cancer or inflammatory disease comprising the antibody or an immunologically active fragment thereof that specifically binds to the NDRG3 epitope consisting of an amino acid sequence set forth in SEQ ID NO: 3

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, since the NDRG3 mediated by lactate generated from a hypoxic response promotes cell proliferation and angiogenesis through a lactate-NDRG3-c-Raf-ERK signaling pathway, a composition including the antibody or an immunologically active fragment thereof that specifically binds to the NDRG3 epitope may be usefully used for preventing and treating a cancer or inflammatory disease.

Also, the present invention provides a kit for diagnosing a cancer or inflammatory disease comprising an antibody or immunologically active fragment thereof, which specifically binds to an NDRG3 epitope consisting of an amino acid sequence set forth in SEQ ID NO: 3, in a test specimen.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

The inflammatory disease includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, since the NDRG3 mediated by lactate generated from a hypoxic response promotes cell proliferation and angiogenesis through a lactate-NDRG3-c-Raf-ERK signaling pathway, the antibody or an immunologically active fragment thereof that specifically binds to the NDRG3 epitope may be usefully used to diagnose a cancer or inflammatory disease.

The kit for diagnosing a cancer or inflammatory disease according to the present invention is preferably used to detect a NDRG3 antigen protein from serum, plasma or blood of a human being, but any specimens expected to include the NDRG3 antigen protein may be all used herein.

Also, the kit for diagnosing a cancer or inflammatory disease according to the present invention includes a secondary antibody conjugate linked with a label that develops a color through reaction with an antigen substrate specific to an antibody specifically binding to the NDRG3 epitope; and at least one selected from the group consisting of a chromogenic substrate solution for developing a color with the label, a washing solution, and an enzymatic reaction stop solution. The secondary antibody is preferably labeled with a conventional chromogenic agent that participates in a chromogenic reaction. In this case, at least one label selected from the group consisting of a fluorescein such as horseradish peroxidase (HRP), alkaline phosphatase, colloid gold, poly L-lysine-fluorescein isothiocyanate (FITC), rhodamine-B-isothiocyanate (RITC), etc., and a dye may be used. Also, a substrate inducing color development is preferably used in response to the label that participates in the chromogenic reaction, and at least one selected from the group consisting of 3,3′,5,5′-tetramethyl benzidine (TMB), 2,2′-azino-bis(3-ethylbenzothiazoline-6-sulfonic acid) (ABTS), and ophenylenediamine (OPD) is preferably used, but the present invention is not limited thereto. In this case, the chromogenic substrate is more preferably provided in a state in which the chromogenic substrate is dissolved in a buffer solution (0.1 M NaAc, pH 5.5). The chromogenic substrate such as TMB is dissolved by HRP used as a label for the secondary antibody conjugate to generate a chromogenic deposit and check a deposition level of the chromogenic deposit with the naked eye, thereby detecting the presence of a protein antigen against an anti-metriptase antibody. The washing solution preferably includes a phosphate buffer solution, NaCl, and Tween 20, and is more preferably a buffer solution (PBST) composed of a 0.02 M phosphate buffer solution, 0.13 M NaCl, and 0.05% Tween 20. After a secondary antibody is allowed to react with an antigen-antibody conjugate after an antigen-antibody binding reaction, a proper amount of the washing solution is applied to a fixed substance to wash the fixed substance 3 to 6 times. A sulfate solution (H₂SO₄) may be preferably used as the reaction stop solution.

Also, the kit for diagnosing a cancer or inflammatory disease according to the present invention may diagnose prognosis of the cancer or inflammatory disease by diagnosing an antigen against an antibody specific to the NDRG3 epitope through the antigen-antibody binding reaction, and the antigen-antibody binding reaction preferably includes at least one selected from the group consisting of an conventional enzyme-linked immunosorbent assay (ELISA), a radioimmnoassay (RIA), a sandwich assay, Western blotting on polyacrylamide gel, an immunoblot assay, and an immunohistochemical staining method, but the present invention is not limited thereto. Also, at least one selected from the group consisting of a well plate synthesized from a nitrocellulose membrane, a PVDF membrane, a polyvinyl resin or a polystyrene resin, and a slide glass made of glass may be used as the fixed substance for the antigen-antibody binding reaction, but the present invention is not limited thereto.

Also, the present invention provides a method of preventing a cancer or inflammatory disease comprising administering a pharmaceutically effective amount of the antibody or an immunologically active fragment thereof, which specifically binds to the NDRG3 epitope consisting of an amino acid sequence set forth in SEQ ID NO: 3, to a subject.

In addition, the present invention provides a method of treating a cancer or inflammatory disease comprising administering a pharmaceutically effective amount of the antibody or an immunologically active fragment thereof, which specifically binds to the NDRG3 epitope consisting of an amino acid sequence set forth in SEQ ID NO: 3, to a subject.

Additionally, the present invention provides an antibody or an immunologically active fragment thereof, which specifically binds to the NDRG3 epitope consisting of an amino acid sequence set forth in SEQ ID NO: 3, for use as a pharmaceutical composition for preventing and treating a cancer or inflammatory disease.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, since the NDRG3 mediated by lactate generated from a hypoxic response promotes cell proliferation and angiogenesis through a lactate-NDRG3-c-Raf-ERK signaling pathway, a composition including the antibody or an immunologically active fragment thereof that specifically binds to the NDRG3 epitope may be usefully used for preventing and treating a cancer or inflammatory disease.

Also, the present invention provides an NDRG3-overexpressing transgenic mouse that is transfected with a vector comprising a promoter, an N-myc downstream-regulated gene 3 (NDRG3) gene, and a polyadenylation sequence.

The NDRG3 protein preferably consists of an amino acid sequence set forth in SEQ ID NO: 1.

The promoter is preferably a cytomegalovirus enhancer/chicken beta-actin promoter (a CAG promoter), but the present invention is not limited thereto.

The polyadenylation sequence is preferably a rabbit β-globin polyadenylation (poly A) sequence, but the present invention is not limited thereto.

The vector is preferably a linear DNA, plasmid DNA or recombinant viral vector, but the present invention is not limited thereto. Also, the recombinant viral vector may include a retrovirus, an adenovirus, a herpes simplex virus, and a lentivirus, but the present invention is not limited thereto.

The transgenic mouse may be defined as an animal that gains new genetic traits through a recombinant DNA technique and germ cell engineering method rather than a traditional crossbreeding technique. That is, transgenesis refers to a process in which a gene a from an animal called ‘A’ does not exist in an animal called ‘b,’ but the gene a is directly transferred to the B animal using the recombinant DNA technique and the germ cell engineering method without undergoing the crossbreeding technique so that the traits, that is, abilities of the gene a can be expressed in the B animal. Such transgenesis is divided into two groups, that is, somatic cell transgenesis and germ line transgenesis. The somatic cell transgenesis refers to a process in which newly gained genetic traits are expressed in the animal, but are not expressed in offspring thereof. In this case, a representative example may be gene therapy in human beings. When any disorder is caused due to abnormality or deficiency of a certain gene, a normal gene is injected into cells of a patient to heal the disorder so that the gene functions normally. In this case, the newly injected gene is expressed in the patient of the day, but not transferred to the offspring. On the other hand, the germ line transgenesis refers to a process in which a new gene is expressed in the patient of the day and is also transferred to the offspring by directly transferring the gene to a germ line or transferring transformant cells to a germ line. In general, true production of a transformant animal is achieved through the germ line transgenesis.

Transgenesis may be mainly divided into two elements: a genetic transgenesis method and a medium (that is, a type of cells) for transferring a modified trait to an animal. First, the genetic transgenesis method includes two methods, for example, a method of randomly transferring a newly injected gene, and a method of y transferring a newly injected gene to a certain site. Such a type of cells for transferring a modified trait to an animal may typically include a fertilized egg. In addition, the type of cells may include sperms, embryonic stem cells, and somatic cells.

A method of producing a transgenic animal may include a pronuclear injection method, a method using a viral vector, a method using embryonic stem cells, a nuclear transplantation method, and a method using sperms.

Also, the present invention provides a transgenic mouse for a cancer or inflammatory disease model, which is transfected with a vector comprising a promoter, an NDRG3 gene, and a polyadenylation sequence.

The NDRG3 protein preferably consists of an amino acid sequence set forth in SEQ ID NO: 1, the promoter is preferably a cytomegalovirus enhancer/chicken beta-actin promoter (a CAG promoter), and the polyadenylation sequence is preferably a rabbit β-globin polyadenylation(poly A) sequence, but the present invention is not limited thereto. Also, the vector is preferably a linear DNA, plasmid DNA or recombinant viral vector, but the present invention is not limited thereto.

According to one specific embodiment of the present invention, the present inventors have selected NDRG3 (SEQ ID NO: 1) from candidate proteins that bind to the PHD2 protein associated with the activity of HIF so as to find an HIF-independent factor involved in hypoxia, injecting a vector, which comprises the CAG promoter, the NDRG3 gene and rabbit β globin polyadenylation sequence, into the pronucleus of a fertilized egg of a C57/BL6 mouse to prepare three NDRG3-overexpressing transgenic C57/BL6 mice TG-2, TG-8 and TG-13 so as to check the molecular biochemical functions of the NDRG3 protein in the hypoxia (see FIGS. 1A to 1C, and FIGS. 3A to 3C).

Also, according to one specific embodiment of the present invention, the present inventors have conducted immunohistochemical analysis using the prepared NDRG3-overexpressing transgenic mice, conducted Western blotting and RT-PCR to determine expression of NDRG3 signaling-related proteins, and found that tumor is observed in various organs such as lungs, intestines, livers, and the like from the NDRG3-overexpressing transgenic mice, and lymphoma-expressing B cells and T cells are observed in secondary lymphoid organs such as mesenteric lymph node and spleen. As a result, the present inventors have found that cell proliferation markers and angiogenic markers are increasingly expressed (see FIGS. 21A to 21E).

Therefore, it is revealed that tumor is formed in tissues from liver, intestines, lungs, etc. of the transgenic mice prepared to overexpress NDRG3, and angiogenesis and cytokine expression increases in the liver tissues. As a result, the NDRG3-overexpressing transgenic mice may be usefully used to conduct onset mechanisms of diseases, such as cancer or inflammatory diseases, caused by hypoxia, screen novel genes involved in the onset mechanisms, and develop therapeutic agents and new pharmaceuticals.

Also, the present invention provides a fertilized egg from a transgenic mouse obtained by injecting a vector comprising a promoter, an NDRG3 gene, and a polyadenylation sequence, into a fertilized egg of a mouse.

The NDRG3 protein preferably consists of an amino acid sequence set forth in SEQ ID NO: 1, the promoter is preferably a cytomegalovirus enhancer/chicken beta-actin promoter (CAG promoter), and the polyadenylation sequence is preferably a rabbit β-globin polyadenylation (poly A) sequence, but the present invention is not limited thereto. Also, the vector is preferably a linear DNA, plasmid DNA or recombinant viral vector, but the present invention is not limited thereto.

Injection of the vector is preferably performed using a microinjection method, but the present invention is not limited thereto. More specifically, the following methods may be used to inject the vector into a fertilized egg of a mouse. First, a pronuclear injection method is a method of microinjecting DNA into a one-cell phase pronucleus or injecting a nucleus into a fertilized egg at a two-cell phase. This method is the most safe and reliable method for transferring a gene, and has advantages of matching infraspecific efficiency regardless of intraspecific variations, injecting a gene regardless of the size of a DNA fragment. However, since transformants are low in yield efficiency and originates from a gene used for transduction, the quality of DNA to be injected should be good, and different effects may be expressed in response to a position of a chromosome into which the gene is inserted. Also, it is important to maintain a proper concentration of DNA to be injected, and the pronucleus into which DNA is injected may be selected from male pronuclei having a larger size than female pronucles. When a foreign DNA substance is injected into the pronucleus, it is advantageous to inject multiple copies of a gene called a concatamer, rather than injecting one copy of the gene since the gene have a higher probability of being fused with DNA. DNA injection is performed at a DNA synthesis phase (S-phase) that is a phase at which the chromosomes are unfolded, and may be performed using a method of increasing a concentration of DNA injected to enhance DNA transfer efficiency, a method of increasing damage of DNA, a method of improving DNA recovery activity, a method of unfolding chromosomes so that a gene is inserted into the chromosomes at a varying temperature, and a method of performing a reverse transcription using a retroviral integrase. Next, there is a method of injecting a gene using a viral system. An adenoviral vector, a retroviral vector, or an adeno-associated viral vector may be used. Among these, the retroviral vector is most widely used herein. A retrovirus has a single-stranded RNA genome, and is present in the form of a provirus in the chromosome of a host cell. A foreign DNA is inserted into this proviral DNA using a reverse transcription function of endogenous retroviruses (ERVs), thereby forming transformant cells. Using this principle, fertilized eggs with a 4- to 8-cell phase are recovered to remove zona pellucidae, cultured for 16 to 24 hours together with cells producing viruses, and transplanted into a surrogate to prepare an animal having a foreign gene. This method has characteristics in that it has high efficiency, it is irreversible when a gene is inserted into the chromosome, the gene may be artificially inserted into a desired site of the chromosome, cells may be partially proliferated in vitro, and a catalytic reaction by a viral enzyme may occur. Also, the method has advantages in that it is technically easier to inject DNA into the pronuclei or nuclei, it is simple to install and handle, and it is possible to accurately establish physiology through insertion of one copy of a target gene. However, the method has drawbacks in that retroviruses should be carefully handled in consideration of stability since the retroviruses are lethal to human bodies, transgenic animals have species specificities, the inserted gene in most of the transgenic animals is transferred to next-generation animals since it is impossible to introduce the gene at an early embryo, and there is a limitation on the size of the introduced gene. When viruses are encapsulated, infection efficiency of the retroviral vector is determined, depending on the probability of interaction between the viruses and a cell membrane, the success or failure of insertion at a mitotic stage, etc. In addition to the method, injection of DNA solution into the cytoplasm, or injection of polylysine/DNA mixture into the cytoplasm may be used, but the present invention is not limited thereto.

Also, the present invention provides a method of preparing an NDRG3-overexpressing transgenic mouse comprising:

1) micro-injecting a vector comprising a promoter, an NDRG3 gene, and a polyadenylation sequence, into a fertilized egg of a mouse;

2) transplanting the fertilized egg into an oviduct to obtain littermates; and

3) determining whether injected DNA is inserted to select a founder mouse from the littermates.

The NDRG3 protein in step 1 preferably consists of an amino acid sequence set forth in SEQ ID NO: 1, the promoter is preferably a CAG promoter, and the polyadenylation sequence is preferably a rabbit β-globin polyadenylation sequence, but the present invention is not limited thereto. Also, the vector is preferably a linear DNA, plasmid DNA or recombinant viral vector, but the present invention is not limited thereto.

Therefore, in the NDRG3 of the present invention, PHD2 binds to a PHD2 docking site of the NDRG3 under a normoxic condition to be ubiquitinated and down-regulated through ubiquitination by a PHD/VHL-mediated pathway, and accumulation of an HIF-1α protein is induced due to inactivity of PHD2 at the beginning of hypoxic condition so that genes (LDHA, PDK1, etc.) involved in metabolic adaptation of cells in response to hypoxia are up-regulated to activate glycolysis. Thereafter, the NDRG3 protein is increasingly expressed by lactate generated/accumulated by the increased glycolysis as well as inhibition of hydroxylation of 294^{th} proline that is a hypoxic target site of NDRG3 due to the PHD2 inactivity under a hypoxic condition. The increasingly expressed NDRG3 serves as a scaffold protein in a sustained hypoxic response to bind to c-Raf and RACK1, and the bound RACK1 recruits PKC-β proteins to form a complex. The c-Raf is then phosphorylated by PKC to activate a c-Raf-ERK1/2 pathway, thereby promoting cell proliferation and angiogenesis (see FIG. 23). Also, tumor is formed in tissues from liver, intestines, lungs, etc. of the transgenic mice prepared to overexpress NDRG3, and angiogenesis and cytokine expression increases in the liver tissues. As a result, the NDRG3-overexpressing transgenic mice may be usefully used to conduct onset mechanisms of diseases, such as cancer or inflammatory diseases, caused by hypoxia, screen novel genes involved in the onset mechanisms, and develop therapeutic agents and new pharmaceuticals.

Also, the present invention provides a method of screening a pharmaceutical composition for preventing and treating a cancer or inflammatory disease comprising:

1) treating the NDRG3-overexpressing transgenic mouse with a candidate material;

2) confirming expression or activity of a NDRG3 protein in a specimen derived from the NDRG3-overexpressing transgenic mouse in step 1; and

3) selecting the candidate material which reduces the expression or activity of the NDRG3 protein in step 2, compared to that in tissues from an untreated control mouse.

The candidate material in step 1 may be an individual antisense nucleotide, short interfering RNA, short hairpin RNA, an aptamer, or an antibody, which is supposed to have a probability of inhibiting the NDRG3 expression or activity or randomly selected according to a conventional selection method, but the present invention is not limited thereto.

The specimen in step 2 preferably includes at least one selected from the group consisting of cells, tissues, blood, sera, saliva, and urine, but the present invention is not limited thereto.

A level of expression or activity of the NDRG3 protein in step 2 is preferably measured using at least one selected from the group consisting of an enzyme-linked immunosorbent assay (ELISA), immunohistochemical staining, Western blotting, and protein chips, but the present invention is not limited thereto.

The cancer preferably includes at least one selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer, but the present invention is not limited thereto.

The inflammatory disease preferably includes at least one selected from the group consisting of asthma, allergic and non-allergic rhinitis, chronic and acute rhinitis, chronic and acute gastritis or enteritis, ulcerative gastritis, acute and chronic nephritis, acute and chronic hepatitis, chronic obstructive pulmonary disease, pulmonary fibrosis, irritable bowel syndrome, inflammatory pain, migraine, headache, backache, fibromyalgia, fascial disorder, viral infection, bacterial infection, fungal infection, burns, wounds from surgical or dental operations, hyperprostaglandin E syndrome, atherosclerosis, gout, degenerative arthritis, rheumatoid arthritis, ankylosing spondylitis, Hodgkin's disease, pancreatitis, conjunctivitis, iritis, peritonitis, uveitis, dermatitis, eczema, and multiple sclerosis, but the present invention is not limited thereto.

According to the present invention, tumor is formed in tissues from liver, intestines, lungs, etc. of the transgenic mouse prepared to overexpress NDRG3, and angiogenesis and cytokine expression increases in the liver tissues. As a result, the NDRG3 may be usefully used for the method of screening a pharmaceutical composition for preventing and treating a cancer or inflammatory disease.

### MODE FOR INVENTION

Hereinafter, exemplary embodiments of the present invention will be described in detail with reference to examples and preparative examples thereof.

However, it should be understood that the following examples and preparative examples are provided herein for the purpose of illustrating the present invention, but intended to limit the scope of the present invention.

### <Example 1> Preparation of NDRG3-Overexpressing Transgenic Mouse

To check an effect of expression of NDRG3 on biochemical characteristics, NDRG3-overexpressing transgenic mice were prepared.

Specifically, a human NDRG3 cDNA sequence (SEQ ID NO: 1) was cloned into a pCAGGS plasmid comprising a CAG-promoter (a cytomegalovirus enhancer and a chicken β-actin promoter) and a rabbit β-globin polyadenylation sequence, and linearized, as shown in the schematic diagram of FIG. 1A. Thereafter, the linearized construct was injected into the pronuclei of fertilized eggs from three C57/BL6 mice. To determine whether a gene is inserted, each of tails of the three C57/BL6 mice was cut, and genomic DNA was extracted from the tails using a mouse tail lysis buffer (60 mM Tris, pH 8.0/100 mM EDTA/0.5% SDS, 500 μg/ml Proteinase K). Then, PCR was performed using primers listed in the following Table 1 to determine a genotype (FIG. 1B). It was confirmed that the transplanted NDRG3 was expressed in each of the three C57/BL6 mice. Then, the C57/BL6 mice were crossbred with a normal mouse to obtain F1-generation mice, whose genotypes were determined in the same manner as described above. As a result, lines of the transgenic mice overexpressing the NDRG3 were established.

TABLE 1

| Primer | | Sequence (5′→3′) |
|---|---|---|
| Human | Forward | AACCATAAATCCTGTTTCAATG (SEQ ID NO: 9) |
| NDRG3 | Reverse | TCCACAACATTGGTTGTCAGG (SEQ ID NO: 10) |

Also, to re-confirm the overexpression of the NDRG3 in the NDRG3-overexpressing transgenic mice TG-2, TG-8 and TG-13, RT-PCR was performed using the primers listed above in Table 1 (FIG. 1C).

### <Example 2> Preparation of Human NDRG3 Antibody

To prepare an antibody for a human NDRG3 protein, an amino acid 32^{nd} to 315^{th} sequence (SEQ ID NO: 2) of the recombinant human NDRG3 protein was cloned into a pET-28a vector, and Escherichia coli (E. coli) strain BL21 was transformed with the pET-28a vector. Thereafter, the transformed E. coli strain BL21 was cultured at 37° C. in an LB medium supplemented with 100 mg/ml ampicillin until an OD value reached OD₅₉₅=0.5, a concentration of 1 mM isopropyl-β-D-thiogalactoside (IPTG) was added thereto to induce the E. coli strain BL21 at 16° C. for 12 hours. Then, the strain was centrifuged at 4° C. for 15 minutes at 4,000 rpm, re-suspended in a buffer [50 mM HEPES (pH 7.5) and 150 mM NaCl], and sonicated for 2 minutes at 3-second pulses in an ice bucket. The strain was again centrifuged at 4° C. for minutes at 12,000 rpm. Subsequently, a recombinant protein was purified by Ni-NTA agarose affinity chromatography using a Ni-NTA agarose resin according to the manufacturer's procedure. Next, the purified recombinant human NDRG3 protein (consisting of 32^{nd} to 315^{th} amino acids) was immunized in a rabbit (New Zealand White). Production of polyclonal antisera was performed by AbFrontier (Seoul, Korea). The antisera were purified by affinity chromatography using an NDRG3 peptide (QNDNKSKTLKCS; consisting of 244^{th} to 255^{th} amino acid: SEQ ID NO: 3) to obtain an anti-NDRG3 antibody. Then, Western blotting was performed to determine specificity of the anti-NDRG3 antibody.

Specifically, a Myc-tagged NDRG1 expression vector, a Myc-tagged NDRG2 expression vector, a Myc-tagged NDRG4 expression vector, and a Myc-tagged NDRG3 expression vector were cloned. Also, to prepare an NDRG3 variant, a site-directed mutation was performed according to the manufacturer's procedure using the Myc-tagged NDRG3 expression vector as a template and using a KOD-Plus-Mutagenesis kit (Toyobo), in which primers were used as listed in the following Table 2, to obtain a Myc-tagged NDRG3 (N66D) variant in which 66^{th} asparagine (Asn, N) of NDRG3 was substituted with aspartic acid. Next, HEK293T cells (ATCC) cultured in a DMEM medium supplemented with 10% FBS (Gibco BRL) and 100 U/ml penicillin (Gibco BRL) were transfected with each of the NDRG1-Myc, NDRG2-Myc, NDRG4-Myc and NDRG3 (N66D)-Myc expression vectors according to the manufacturer's procedure using lipofectamine (Invitrogen). Then, the transfected cells were cultured at 37° C. for 24 hours in a CO₂ incubator (Sanyo), and collected. Subsequently, the collected cells were lysed in a lysis buffer [1% Triton X-100, 150 mM NaCl, 100 mM KCl, 20 mM HEPES (pH 7.9), 10 mM EDTA, and a protease inhibitor cocktail (Roche)], a protein lysate (30 μg) of the cells was electrophoresed on 9% SDS-PAGE, and transferred to a nitrocellulose membrane (PALL Life Sciences). Then, the obtained anti-NDRG3 antibody was treated with a primary antibody to react with each other, and a HRP-conjugated secondary antibody was connected with the primary antibody attached to the membrane. The resulting conjugate was confirmed by ECL (Pierce Chemical Co, USA) (FIG. 2).

TABLE 2

| Primer | | Sequence (5′→3′) |
|---|---|---|
| NDRG3 | Sense | GACCATAAATCCTGTTTCAATGCATTCTTT |
| (N66D) | | (SEQ ID NO: 11) |
| | Antisense | GAGGCCAATGTCATGATATGTTAGTATAAC |
| | | (SEQ ID NO: 12) |

As a result, it was revealed that the prepared anti-NDRG3 antibody bound to the NDRG3 (N66D) variant other than the NDRG1, NDRG2 and NDRG4 variants, as shown in FIG. 2. Accordingly, the anti-NDRG3 antibody was an antibody that bound the NDRG3 antibody and variants to an antigen, and thus was used to determine the molecular biological functions of the NDRG3 according to the present invention (FIG. 2).

### <Example 3> Confirmation of NDRG3 as PHD2 Binding Protein

It was reported that prolyl-hydroxylase domain 2 (PHD2) regulates the activity of hypoxia-inducible factor-1α (HIF-1α), which is an important transcription factor for expression of genes induced under a hypoxic condition, under a normoxic condition (Wenger, R. H. et al., Curr. Pharm. Des., 2009(15), 3886-3894). Therefore, to screen factors that are regulated by PHD2 in an HIF-independent manner in response to a hypoxic response, an immunoprecipitation assay, an immunostaining assay and a micro-LC-MS/MS assay were performed.

Specifically, to screen a PHD2 binding protein, a construct coding for Flag-tagged PHD2 was prepared, as shown in the schematic diagram of FIG. 3A. Thereafter, MCF-7 cells (ATCC) cultured in a DMEM medium supplemented with 10% FBS (Gibco BRL) and 100 U/ml penicillin (Gibco BRL) were transfected with the construct and the control (Mock) according to the manufacturer's procedure using lipofectamine (Invitrogen). Then, the transfected cells were treated with a proteasome inhibitor MG132 (10 μM), left under a hypoxic condition for 24 hours in a O₂/CO₂ incubator (Sanyo) containing a mixed gas composed of 92 to 94% N₂, 5% CO₂ and 1% O₂, and then lysed in a lysis buffer [1% Triton X-100, 150 mM NaCl, 100 mM KCl, 20 mM HEPES (pH 7.9), 10 mM EDTA, and a protease inhibitor cocktail (Roche)]. A protein lysate (1 mg) of the cells was reacted overnight at 4° C. using an anti-FLAG M2 affinity gel (Sigma), centrifuged, immunoprecipitated, and then electrophoresed on 9% SDS-PAGE. Then, the protein lysate was stained with Coomassie Brilliant Blue (CBB). Subsequently, a protein band having a different immunoprecipitation pattern in the PHD2-Flag sample was isolated from an SDS-PAGE gel, and the isolated gel was digested with trypsin for micro-LC-MS/MS analysis. The digested proteins were injected into a fused silica capillary column (having am inner diameter of 100 mm and an outer diameter of 360 mm), including a 5-mm particle size Aqua C18 reverse-phase column having a size of 8 cm. The column was transferred to an Agilent HP 1100 quaternary LC pump, and peptides were separated at a flow rate of 250 nl/minute using a separation system. Also, a buffer A (5% acetonitrile and 0.1% formic acid) and a buffer B (80% acetonitrile and 0.1% formic acid) were used for 120-minutes gradients. The eluted peptides were separated at a potential of 2.3 kV DC by an electrospray method using an LTQ linear ion trap mass spectrometer (Thermo Finnigan). One data-dependent scan composed of the whole MS scan (400 to 1,400 m/z), and five data-dependent MS/MS scans were used to generate MS/MS spectra of the eluted peptides. Then, the MS/MS spectra were analyzed from the database on NCBI human protein sequences using Bioworks version 3.1. DTASelect was used to filter search results, and Xcorr values were applied to different charge states of the peptides: 1.8 was applied to singly charged peptides, 2.5 was applied to doubly charged peptides, and 3.5 was applied to triply charged peptides (FIG. 3B).

As a result, as shown in FIG. 3B, it was revealed that ten PHD2 binding proteins were screened from the proteins extracted from the PHD2-Flag sample through mass spectrometry. Among these, NDRG3 belonging to a gene family associated with cell proliferation, migration and invasion as well as differentiation, development and hypoxia was selected (FIG. 3B).

Also, to further determine whether the NDRG3 is a PHD2 binding protein, an immunoprecipitation assay and Western blotting were performed.

Specifically, an MCF-7 cell lysate transfected with the control and the Flag-tagged PHD2 construct was immunoprecipitated using an anti-FLAG M2 affinity gel (Sigma), electrophoresed on 9% SDS-PAGE, and then transferred to a nitrocellulose membrane (PALL Life Sciences). Thereafter, an anti-NDRG3 antibody and an anti-Flag antibody were treated with a primary antibody to react with each other, and a HRP-conjugated secondary antibody was connected with the primary antibody attached to the membrane. The resulting conjugate was confirmed by ECL (Pierce Chemical Co, USA) (FIG. 3C).

As a result, as shown in FIG. 3C, it was revealed that the NDRG3 was a PHD2 binding protein by confirming the presence of the 42 KDa protein isolated from the mmunoprecipitated FLAG beads (FIG. 3C).

### <Example 4> Confirmation of NDRG3 as Innate Substrate for PHD2

<4-1> Confirmation of Interaction Between PHD2 and NDRG3

To check an effect of the PHD2 binding protein on interaction between PHD2 and NDRG3 and an effect of the interaction between PHD2 and NDRG3 on expression of the NDRG3 protein, an immunoprecipitation assay, Western blotting, and an in vitro pull-down assay were performed.

Specifically, HeLa cells (ATCC) cultured in a DMEM medium supplemented with 10% FBS (Gibco BRL) and 100 U/ml penicillin (Gibco BRL) were transfected with the construct coding for Flag-tagged PHD2 as described in Example 3, maintained for 24 hours under a hypoxic condition (1% oxygen), and then lysed. Thereafter, the resulting cell lysate was immunoprecipitated using anti-FLAG M2 beads, and then subjected to Western blotting using the anti-NDRG3 and anti-Flag antibodies as a primary antibody (Upper panel of FIG. 4A). Also, PHD2 was cloned into a recombinant pET-28a plasmid using the method disclosed in Example 1, transformed into E. coli strain BL21, and then purified using a Ni-NTA agarose resin. Also, NDRG3 was cloned into a recombinant pGEX-4T-2 plasmid, transformed into E. coli strain BL21, and then purified using a GST-conjugated agarose resin (ELPIS BIOTECH, Korea). Then, 10 μg of the recombinant His-PHD2 protein and/or 10 μg of the recombinant GST-NDRG3 protein were incubated at 4° C. for 4 hours with a Ni-NTA agarose (Giagen) resin at a final concentration of 0.2 mg/ml. Subsequently, the NDRG3 protein bound to the resin was treated with an SDS sample buffer solution, electrophoresed on SDS-PAGE as described in Example 3, and then subjected to Western blotting using the anti-NDRG3 antibody as a primary antibody (Lower panel of FIG. 4A).

Also, to determine the functional relationship between PHD2 and NDRG3, MCF-7 cells (FIG. 4B) or HeLa cells (FIG. 4C) were treated with a varying concentration of desferrioxamine (DFX), which was a PHD2 inhibitor, for 24 hours under a normoxic condition (21% O₂), and then lysed, as described in Example 3. The cell lysate was subjected to Western blotting using anti-NDRG3, anti-HIF-1α, and anti-β-actin (FIGS. 4B and 4C).

As a result, as shown in FIG. 4A, it was revealed that the immunoprecipitated PHD2 and NDRG3 were bound to each other under a hypoxic condition, and the recombined PHD2 and NDRG3 were bound to each other, indicating that the PHD2 and NDRG3 directly interacted with each other (FIG. 4A).

Also, as shown in FIGS. 4B and 4C, it was revealed that the NDRG3 protein was slightly expressed at a background level in the HeLa cells, but the NDRG3 protein was increasingly expressed when the activity of PHD2 was inhibited by the PHD2 inhibitor, DFX, in both the MCF-7 and HeLa cells. Accordingly, it was revealed that the NDRG3 was accumulated in a drug-dependent manner as the PHD2 activity was inhibited, and thus the NDRG3 was an innate substrate for PHD2 (FIGS. 4B and 4C).

<4-2> Confirmation of Interaction Between PHD Family Proteins and NDRG3

A PHD family is composed of PHD1, PHD2, PHD3, P4HTM, and P4HA1, and these family members are reported to play an important role in regulation of the HIF proteins (Wenger, R. H. et al., Curr. Pharm. Des., 2009(15), 3886-3894). Therefore, to determine the relevance between the NDRG3 and VHL, which is a target protein of an E3 ubiquitin ligase complex, and the other PHD family members other than the PHD2, RT-PCR, an immunoprecipitation assay and Western blotting were performed using cells in which PHD was knocked down due to RNA interference, and cells in which the PHD family was overexpressed.

Specifically, Samchully Pharm Co., Ltd (Korea) was asked to construct siRNA using sequences as listed in the following Table 3. Thereafter, HeLa cells were transfected with the siRNA and siVHL (siGENOME SMART pool, Dharmacon) according to the manufacturer's procedure using lipofectamine to obtain cells in which expression of GFP, PHD1, PHD2, PHD3, P4HTM, P4HA1 or VHL was inhibited. Then, each of the cells in which the expression was inhibited was maintained and cultured for 48 hours under a normoxic condition (21% O₂), and then collected. Subsequently, total RNA was extracted from the collected cells using a Trizol reagent (Invitrogen, Carlsbad, Calif.). Then, 5 μg of the extracted RNA was reacted with a reverse transcriptase to synthesize cDNAs, and PCR products were electrophoresed on an agarose gel to be visualized (FIG. 5A).

TABLE 3

| siRNA | | Sequence (5′→3′) |
|---|---|---|
| GFP | Sense | GUUCAGCGUGUCCGGCGAGTT |
| | | (SEQ ID NO: 13) |
| | Antisense | CUCGCCGGACACGCUGAACTT |
| | | (SEQ ID NO: 14) |
| PHD1 | Sense | CAUCGAGCCACUCUUUGACTT |
| | | (SEQ ID NO: 15) |
| | Antisense | GUCAAAGAGUGGCUCGAUGTT |
| | | (SEQ ID NO: 16) |
| PHD2 | Sense | AACGGGUUAUGUACGUCAUTT |
| | | (SEQ ID NO: 17) |
| | Antisense | AUGACGUACAUAACCCGUUTT |
| | | (SEQ ID NO: 18) |
| PHD3 | Sense | CCAGAUAUGCUAUGACUGUTT |
| | | (SEQ ID NO: 19) |
| | Antisense | ACAGUCAUAGCAUAUCUGGTT |
| | | (SEQ ID NO: 20) |
| P4HTM | Sense | GAGUGUCGGCUCAUCAUCCTT |
| | | (SEQ ID NO: 21) |
| | Antisense | GGAUGAUGAGCCGACACUCTT |
| | | (SEQ ID NO: 22) |
| P4HA1 | Sense | GAUCUGGUGACUUCUCUGATT |
| | | (SEQ ID NO: 23) |
| | Antisense | UCAGAGAAGUCACCAGAUCTT |
| | | (SEQ ID NO: 24) |

Also, to check the interaction between the PHD family and the NDRG3 protein, Flag-tagged PHD1, Flag-tagged PHD2, Flag-tagged PHD3, Flag-tagged P4HTM, a Flag-tagged P4HA1 expression vector, and an NDRG3 expression vector were constructed, and HeLa cells were transfected with the NDRG3 and the Flag-tagged PHD family, respectively, using the method described in Example 3. Therefore, the HeLa cells were treated with 20 μM MG132 for 8 hours under normal conditions, harvested, and lysed. Subsequently, the cell lysate was immunoprecipitated with anti-FLAG M2 beads, and was then subjected to Western blotting using the anti-NDRG3 antibody (FIG. 5B).

As a result, as shown in FIGS. 5A and 5B, it was confirmed that the NDRG3 was accumulated when the expression of PHD2 and VHL was inhibited due to the RNA interference. Accordingly, it was revealed that the PHD2 in the PHD family group was a main posttranslational regulator in stabilizing the NDRG3 protein, and the NDRG3 was a target protein of ubiquitin under a normoxic condition, indicating that the NDRG3 was a substrate in a PHD2/VHL-mediated posttranslational process (FIGS. 5A and 5B).

<4-3> Determination of PHD2 Docking Sites in NDRG3

An immunoprecipitation assay and a protein docking simulation were performed to determine a docking site of PHD2 in the NDRG3 as a substrate of PHD2, and an immunoprecipitation assay and Western blotting were performed on NDRG3 variants, in which docking sites were mutated using site-directed mutations, to determine a binding affinity between PHD2 and various supposed PHD2-docking sites of the NDRG3 determined through the docking simulation.

Specifically, a protein-protein docking simulation of a supposed target protein was performed using HEX6.3 (D. W. Ritchie. et al., Genet, 2000(39), 178-194). For calculation select options, shapes and electrostatistics were selected as correlation types, and bumps and volumes were selected after a process. Default configuration was used as the other select option. For single target docking (that is, NDRG3-EGLN1), the simulation was performed once for the listed select options. Docking for multiple target structures (that is, NDRG3 and PHD2) was performed using a two-step experiment. In the first step, NDRG3 was used as a receptor protein in docking experiments for respective targets. In the second step, a protein-protein interaction product was used as a receptor protein for docking other proteins. The input order for the second experiment was used for PHD2. Docking calculation was performed with a root-mean-square deviation (RMSD), and the results were filtered at a level at which the calculated values from a single input matched the calculated values from multiple inputs. Among the filtered result values, the most stable one was selected using the total HEX6.3 score (the sum of shape scores and electrostatistics cores) (FIG. 6A).

Also, to check the binding affinity of PHD2 to the docking sites of NDRG3 confirmed through the docking simulation, a site-directed mutation was performed according to the manufacturer's procedure using a KOD-Plus-Mutagenesis kit (Toyobo) using an NDRG3-Myc expression vector as a template, thereby obtaining a Myc-tagged NDRG3 (R47D) variant in which 47^{th} arginine (Arg, R) of the NDRG3 was substituted with aspartic acid (Asp, D), a Myc-tagged NDRG3 (N66D) variant in which 66^{th} asparagine (Asn, N) of the NDRG3 was substituted with aspartic acid, a Myc-tagged NDRG3 (Q97E) variant in which 97^{th} glutamine (Gln, Q) of the NDRG3 was substituted with glutamic acid (Glu, E), and a Myc-tagged NDRG3 (V296D) variant in which 296^{th} valine (Val, V) of the NDRG3 was substituted with aspartic acid. Thereafter, HEK293T cells (ATCC) were transfected with each of the Myc-tagged NDRG3 variants, the Flag-tagged PHD2, and the HA-tagged VHL construct at the same time, as described in Example 3, and the transformed HEK293T cells were treated with 20 μM MG132 for 8 hours, and then lysed. The cell lysate was immunoprecipitated using an anti-Myc affinity gel (Sigma), and then subjected to Western blotting using the anti-Flag, anti-HA and anti-Myc antibodies as primary antibodies (FIG. 6B).

As a results, as shown in FIG. 6A, it was confirmed that 47^{th} arginine, 66^{th} asparagine, 68^{th} lysine, 69^{th} serine, 72^{nd} asparagine, 73^{rd} alanine, 76^{th} asparagine, 77^{th} phenylalanine, 78^{th} glutamic acid, 81^{st} glutamine, 97^{th} glutamine, 98^{th} glutamine, 99^{th} glutamic acid, 100^{th} glycine, 101^{st} alanine, 102^{nd} proline, 103^{rd} serine, 203^{rd} leucine, 204^{th} aspartic acid, 205^{th} leucine, 208^{th} threonine, 209^{th} tyrosine, 211^{th} methionine, 212^{th} histidine, 214^{th} alanine, 215^{th} glutamine, 216^{th} aspartic acid, 217^{th} isoleucine, 218^{th} asparagine, 219^{th} glutamine, 296^{th} valine, 297^{th} valine, 298^{th} glutamine, 300^{th} glycine, and 301^{st} lysine residues were supposed to be PHD2-docking sites of the NDRG3 through a docking model between the known PHD2 substrate (Structure. 2009(7), 981-989) and NDRG3 substrate, and the amino acid residue at positions 47, 66, 97 and 296 of the PHD2-docking sites were more important (FIG. 6A).

Also, as shown in FIG. 6B, it was confirmed that the NDRG3 (V296D) variant and the NDRG3 (Q97E) variant bound to PHD2, but the NDRG3 variant in which the amino acid at position 47 or 66 of the NDRG3 was mutated did not bind to PHD2, and also that a large amount of the NDRG3 variants having high affinity to PHD2 was immunoprecipitated with VHL. As a result, it was revealed that the NDRG3 variants showed various PHD2-binding affinities (V296D>Q97E>R47D≈N66D), and the amino acid residues at positions 47 and 66 of the NDRG3 were important for docking of PHD2 under a normoxic condition, and were involved in VHL (FIG. 6B).

<4-4> Confirmation of Regulation of NDRG3 Through PHD2/VHL-Mediated Proteasome Pathway Under Normoxic Condition

To check an effect of VHL, which is a target element of a PHD2/E3 ubiquitin ligase complex, on expression and regulation of the NDRG3 protein under a normoxic condition, the proteasome activity was inhibited using MG132, and Western blotting and an in vivo ubiquitination assay were performed.

Specifically, a vector for transfection (an MSCV retroviral system) was constructed using a full-length NDRG3 cDNA (SEQ ID NO: 1) and a pMSCVneo retroviral vector (Clontech). To construct the viral vector, a GP293 cell line was transfected using lipofectamine (Invitrogen). After 48 hours, HeLa cells were treated with a cell supernatant containing NDRG3-retroviruses or control-retroviruses together with 6 μg/ml of polybrene for 24 hours. Then, the cells in which the NDRG3 was overexpressed were untreated or treated with 20 μM MG132 for 8 hours, and then collected. The collected cells were subjected to Western blotting using an anti-NDRG3 antibody and an anti-β-actin antibody (FIG. 7A).

Also, to determine ubiquitination of NDRG3 under a normoxic condition, an in vivo ubiquitination assay was performed. First, a vector for transfection was constructed using shNDRG3 (Sigma-Aldrich, SEQ ID NO: 4) and a lentiviral vector to inhibit expression of the NDRG3. To construct the viral vector, a packing cell line was transfected as described above. Thereafter, HeLa cells were treated with a cell supernatant containing the NDRG3 shRNA-expressing lentiviruses together with 6 μg/ml of polybrene, and kept in a DMEM medium supplemented with 10% FBS. Then, the control HeLa cells, the HeLa cells in which the NDRG3 expression was inhibited, and the HeLa cells in which the NDRG3 was overexpressed were transfected with HA-tagged ubiquitin, as described in Example 3, treated with 20 μM MG132 for 8 hours, and collected. Then, the collected cells were lysed. Subsequently, the cell lysate was precleared by adding 30 μl of protein G-agarose beads (Santa Cruz Biotechnology), and then immunoprecipitated with an anti-NDRG3 antibody. A polyubiquitinated form of NDRG3 was confirmed through Western blotting using an anti-HA antibody (FIG. 7B).

As a result, as shown in FIGS. 7A and 7B, it was confirmed that, when the proteasome was inhibited with MG132 under a normoxic condition, the NDRG3 protein was actively ubiquitinated in the cells in which the NDRG3 was overexpressed, and thus it was confirmed that the NDRG3 was ubiquitinated under the normoxic condition, and then degraded through a proteasome pathway (FIGS. 7A and 7B). Accordingly, based on the results obtained in Example 4, it was revealed that the NDRG3 was an intrinsic PHD2-interacting protein, and the expression of the NDRG3 protein was posttranslationally regulated through a PHD2/VHL-mediated proteasome pathway.

### <Example 5> Confirmation of Expression of NDRG3 Under Hypoxic Condition

<5-1> Confirmation of Oxygen-Dependent Expression of NDRG3 Protein

Since the activity of PHD2 was dependent on the O₂ availability, various types of cells were subjected to an immunoprecipitation assay, Western blotting, an immunofluorescent staining assay, and an in vivo ubiquitination assay under different oxygen conditions to check an effect of the oxygen conditions on stability of the NDRG3 protein that is an innate substrate of PHD2.

Specifically, MCF-7 cells cultured in a DMEM medium supplemented with 10% FBS and 100 U/ml penicillin were kept by time in an O₂/CO₂ incubator containing a mixed gas composed of 1%, 3%, 5% and 21% O₂ and 92 to 94% N₂, and 5% CO₂, and then collected. Thereafter, the collected cells were lysed, as described in Example 3, and the cell lysate was subjected to Western blotting using anti-NDRG3 and anti-β-actin to determine expression of the NDRG3, the levels of which were plotted in a graph (FIG. 8A).

Also, MCF-7 cells cultured on cover slips in a DMEM medium supplemented with 10% FBS and 100 U/ml penicillin were kept under a hypoxic condition by time in an O₂/CO₂ incubator containing a mixed gas composed of 1% O₂, 94% N₂ and 5% CO₂. Thereafter, the cells were fixed in 4% paraformaldehyde/PBS for 20 minutes, permeated with 0.3% TritonX-100/PBS at room temperature for 5 minutes, and then cultured in a blocking solution (PBS supplemented with 1% BSA) for 30 minutes. Then, the cells were reacted with an anti-NDRG3 antibody (1/1,000) at room temperature for an hour, washed, and then reacted with a secondary antibody [Alexa Flour 488-conjugated goat anti-rabbit IgG (1/1,000), or Alexa Flour 594-conjugated goat anti-mouse IgG (1/1,000); Amersham] and DAPI (3 μM, Sigma). Subsequently, the cells were visualized using a Zeiss LSM 510 confocal microscope (FIG. 8B).

Also, MCF-7 (breast), PLC/PRF/5 (liver), Huh-1 (liver), HeLa (cervix), HEK293T (kidney) and MCF-10A (breast) cells cultured in a DMEM medium supplemented with 10% FBS and 100 U/ml penicillin, and SW480 (large intestine) and IMR-90 (lung) cells cultured in an RPMI 1640 medium supplemented with 10% FBS and 100 U/ml penicillin were kept under a hypoxic condition (1% O₂) by time in a O₂/CO₂ incubator, as described above, and collected. Thereafter, the collected cells were lysed, as described in Example 3, and the cell lysate was subjected to Western blotting using anti-NDRG3 and anti-β-actin to determine expression of the NDRG3, the levels of which were plotted in a graph (FIG. 8C).

In addition, an in vivo ubiquitination assay was performed to determine ubiquitination of the NDRG3 under a hypoxic condition. HeLa cells were transfected with HA-tagged ubiquitin and Myc-tagged NDRG3, as described in Example 3, cultured for 40 hours under a normoxic condition, and then treated with 20 μM MG132 for 8 hours. Thereafter, the cells were further cultured for 24 hours under a hypoxic condition (1% O₂), collected, and then lysed. Then, the cell lysate was precleared by adding 30 μl of protein G-agarose beads (Santa Cruz Biotechnology), immunoprecipitated with an anti-Myc antibody, and then subjected to Western blotting using an anti-HA antibody (FIG. 8D).

As a result, as shown in FIGS. 8A and 8B, it was confirmed that the NDRG3 protein was increasingly accumulated when the O₂ concentration in the cells decreased and over time, and that the NDRG3 protein was accumulated in inverse proportion to the O₂ concentration (FIGS. 8A and 8B).

Also, as shown in FIG. 8C, it was confirmed that the accumulation of the NDRG3 protein in response to the hypoxic condition was identical in cancer cells derived from various tissues such as large intestines, livers, cervix, kidney, lungs, etc., as well as non-transformed cells, and that the inverse relationship between the O₂ concentration and the NDRG3 protein was proven to be a typical phenomenon (FIG. 8C).

Further, as shown in FIG. 8D, it was confirmed that the ubiquitination of the NDRG3 protein decreased under the hypoxic condition (FIG. 8D). Accordingly, based on the results, it was revealed that the accumulation and ubiquitination of the NDRG3 protein increased and decreased under the hypoxic condition, respectively, unlike the normoxic condition.

<5-2> Confirmation of Stability of NDRG3 Protein in Response to Oxygen Conditions

To check a change in expression of the NDRG3 protein in response to hypoxic condition and a normoxic condition, the cells were subjected to Western blotting under different oxygen conditions.

Specifically, to determine expression of the NDRG3 protein under a sustained hypoxic condition, the MCF-7 cells statically cultured by time under a hypoxic condition (1% O₂), and the MCF-7 cells statically cultured under a normoxic condition (21% O₂), as described in Example 5-1, were collected, lysed, as described in Example 3, and then subjected to Western blotting using anti-NDRG3, anti-HIF-1α and anti-β-actin antibodies, the values of which were plotted in a graph (FIG. 9A).

Also, to check a change in expression of the NDRG3 protein when the hypoxic condition returned to the normoxic condition, the MCF-7 cells statically cultured for 24 hours under a hypoxic condition (1% O₂), as described in Example 5-1, and the MCF-7 cells statically cultured by time under a normoxic condition (21% O₂) were collected, lysed, as described in Example 3, and then subjected to Western blotting using anti-NDRG3, anti-HIF-1α and anti-β-actin antibodies, the values of which were plotted in a graph (FIG. 9B).

As a result, as shown in FIGS. 9A and 9B, it was confirmed that the expression of the HIF-1α protein was induced at the beginning of hypoxia, and was reduced as the hypoxia lasted, but the expression of the NDRG3 protein was continuously maintained to the end of hypoxia. Also, it was confirmed that the NDRG3 expression continuously induced under a hypoxic condition was slowly eliminated until the condition of the cells returned again to the normoxic condition (FIGS. 9A and 9B).

<5-3> Confirmation of Stability Regulatory Mechanism of NDRG3 Protein in Response to Oxygen Conditions

To determine target sites of the NDRG3 protein under a hypoxic condition, a micro-LC-MS/MS assay was performed, and Western blotting was performed for the cells in which the NDRG3 variants prepared through the site-directed mutation were overexpressed. Also, to determine the relevance between the hypoxic target sites of the NDRG3 protein and PHD2/VHL under a normoxic condition, an immunoprecipitation assay and Western blotting were performed.

Specifically, to determine the target sites of the NDRG3 protein under a hypoxic condition, the HeLa cells in which the NDRG3 prepared by the method disclosed in Example 4-2 was overexpressed were treated with 20 μM MG132, collected, and then lysed. Thereafter, the cells were immunoprecipitated with an anti-Myc affinity gel, as described in Example 3, and then subjected to a micro-LC-MS/MS assay (FIG. 10A).

Also, to determine binding of PHD2/VHL to the target sites of the NDRG3 protein determined based on the results of the micro-LC-MS/MS assay, first, a site-directed mutation was performed, as described in Example 4-3, to substitute 294^{th} proline (Pro, P) expected as a target site of the NDRG3 protein target site with alanine (Ala, A), thereby preparing a Myc-tagged NDRG3 P294A variant. Thereafter, HEK293T cells were transfected with the NDRG3 variant or a wild-type NDRG3 construct, as described in Example 3, and then cultured for 40 hours under a normoxic condition (21% O₂). Then, the cells which were untreated or further treated with 20 μM MG132 for 8 hours were collected, respectively. The collected cells were lysed, and subjected to Western blotting using anti-Myc and anti-β-actin antibodies (Upper panel of FIG. 10B). Also, HEK293T cells were transfected with the Flag-tagged PHD2, HA-tagged VHL, and the Myc-tagged NDRG3P294A variants or the Myc-tagged NDRG3 at the same time, as described in Example 3, cultured for 40 hours under a normoxic condition (21% O₂), further treated with 20 μM MG132 for 8 hours, and then collected. Subsequently, the collected cells were lysed, immunoprecipitated with an anti-Myc affinity gel, and subjected to Western blotting using anti-Flag, anti-HA and anti-Myc antibodies (Lower panel of FIG. 10B).

As a result, as shown in FIG. 10A, it was confirmed through mass spectrometry that the 294^{th} proline residue of the NDRG3 was a PHD2-mediated hypoxic target site, which was then hydroxylated in an oxygen-dependent manner (FIG. 10A).

Also, as shown in FIG. 10B, it was confirmed that, when the NDRG3 variant in which the 294^{th} amino acid residue of the NDRG3 expected to be a hypoxic target site was substituted with alanine was overexpressed, the NDRG3 protein variant was increasingly accumulated under the normoxic condition (Upper panel of FIG. 10B), and the binding of PHD2 and VHL was also reduced (Lower panel of FIG. 10B). As a result, it was revealed that the target sites of the NDRG3 protein interacted with PHD2/VHL under the normoxic condition, and its interaction was regulated by PHD2 in an oxygen-dependent manner (FIG. 10B).

<5-4> Confirmation of Regulation of HIF-Independent NDRG3 Expression in Response to Oxygen Conditions

To determine whether the HIF protein is involved in the regulation of the NDRG3 expression in response to oxygen conditions, the cells were subjected to Western blotting and RT-PCR under different oxygen conditions.

Specifically, to check the expression of the HIF protein in response to oxygen conditions, the MCF-7 cells statically cultured for 24 hours under the normoxic condition (21% O₂), as described in Example 5-2, and the MCF-7 cells statically cultured by time under the hypoxic condition (1% O₂) were collected, and a half of the collected cells were subjected to Western blotting using anti-HIF-1α, anti-HIF-2α and anti-β antibodies, as described in Example 3, to check expression of the proteins. The other half of the collected cells were subjected to RT-PCR, as described in Example 4-2, to check expression of mRNA (FIG. 11A).

Also, Western blotting was performed to check expression of the NDRG3 protein in response to inhibition of HIF and PHD2. First, to inhibit expression of HIF-1α or HIF-2α, a vector for transfection was constructed using shHIF-1α (Sigma-Aldrich), shHIF-2α (Sigma-Aldrich) or control shGFP, and a lentiviral vector, as disclosed in Example 4-4. Thereafter, PLC/PRF/5 cells were treated with a cell supernatant containing the shHIF-1α or shHIF-2α expression lentiviruses together with 6 μg/ml of polybrene, and then kept in a DMEM medium supplemented with 10% FBS. Then, the control PLC/PRF/5 cells, and the PLC/PRF/5 cells in which the HIF-1α or HIF-2α expression was inhibited were treated with 1 mM desferrioxamine (DFX) as a PHD2 inhibitor by time under a normoxic condition (21% O₂), collected and lysed, as described in Example 3. Subsequently, the lysate was subjected to Western blotting using anti-NDRG3, anti-HIF-1α, anti-HIF-2α and anti-β-actin antibodies (FIG. 11B).

In addition, to determine expression of the NDRG3 protein in response to deletion of HIF and VHL, MCF-7 (HIF-1^{+/+} and VHL^{+/+}) cells, and 786-O (HIF-1^{-/-} and VHL^{-/-}) cells in which HIF-1α and VHL loci were genetically blocked were cultured for 24 hours under a hypoxic condition (1% O₂), as described in Example 5-2, and collected and lysed, as described in Example 3. Then, the cell lysate was subjected to Western blotting using anti-NDRG3, anti-HIF-1α and anti-β-actin antibodies (FIG. 11C).

As a result, as shown in FIG. 11A, it was confirmed that an expression level of NDRG3 mRNA was continuously maintained at the beginning of hypoxic condition without any changes as the hypoxic condition lasts regardless of a significant increase in expression of the HIF-1α protein (FIG. 11A).

Also, as shown in FIG. 11B and FIG. 11C, it was confirmed that the accumulation of the NDRG3 protein increased under the normoxic condition as the inhibition of PHD2 activity lasted without being affected by the deletion of HIF (FIG. 11B), and also that the expression of the NDRG3 protein was preserved even under the hypoxic condition without being affected by the deletion of HIF and VHL (FIG. 11C). Accordingly, based on the results of Example 5, it was confirmed that the expression of the NDRG3 protein was regulated by PHD2 in an oxygen-dependent manner, and the NDRG3 expression lasted as the hypoxic condition lasted, indicating that the expression of the NDRG3 protein and mRNA was not directly affected by the HIF activity. Therefore, it was revealed that the NDRG3 played an important role in a hypoxic response lasting in a HIF-independent manner.

### <Example 6> Determination of NDRG3 as Regulator for Sustained Hypoxic Response

<6-1> Confirmation of Functions of NDRG3 Under Hypoxic Condition

To check functions of the NDRG3 in a hypoxic response, the microarray-based transcriptome data on the cells in which the expression of the NDRG3 was inhibited were subjected to gene expression profiling using a gene set analyzer (GAzer) assay.

Specifically, Huh-7 cells in which expression of NDRG3 or HIF-1α was inhibited were prepared using shNDRG3 and shHIF-1α according to the method described in Example 4-4 and Example 5-4, statically cultured by time under a hypoxic condition (1% O₂), as described in Example 5-2, and then collected. Thereafter, total RNA was extracted from the collected cells according to the manufacturer's procedure using an RNA separation kit (RNeasy midi-prep, Qiagen). For microarray analysis, 200 ng of the extracted total RNA was then amplified using an Illumina TotalPrep™ RNA amplification kit, and 700 ng of the amplified cRNA was hybridized with HumanHT-12 v3/v4 expression bead chips (Expression BeadChip) at 58° C. for 16 hours. After washing and staining, the bead chips were scanned using an Illumina BeadArray reader and Bead Scan software (Illumina). Expressed genes were divided based on their functions using gene ontology (GO) analysis (Ashburner, M. et al., Nat. Genet., 2000(25), 25-29). In this process,

Z score (standard value) conversion was used to calculate a deviation score standardized by each gene grouping. A Z score value represents an activity in a GO biological process (FIG. 12A).

Also, HeLa cells were transfected with the NDRG3 (N66D) variant, as described in Example 3, and 136 genes whose expression increased by 1.5 folds compared to the control, 1,535 genes expression increased by 1.5 folds under a hypoxic condition compared to that under a normoxic condition, and 68 genes whose expression commonly increased upon overexpression of the NDRG3 (N66D) variant and under the hypoxic condition were selected, and Z scores (standard values) for biological ontologies in which the selected genes were divided based on their functions through the gene set analyzer (GAzer) assay were plotted in a diagram to examine the functions of the selected genes (FIG. 12B).

As a result, as shown in FIG. 12A, it was confirmed that the NDRG3 deletion statistically showed a significant change in expression level in a functional gene group under a 24-hour hypoxic condition, and the hypoxic functions most affected by the NDRG3 deletion were proven to be angiogenesis and cell proliferation, and glycolysis was one of the hypoxic functions least affected by the NDRG3 deletion. On the other hand, it was confirmed that one of the hypoxic functions most affected by the HIF-1α deletion was glycolysis, and angiogenesis and cell proliferation were least affected by the HIF-1α deletion (FIG. 12A).

Also, as shown in FIG. 12B, it was confirmed that, when the NDRG3 N66D variant in which the docking sites of PHD2 were mutated was overexpressed under a normoxic condition, the NDRG3 N66D variant was up-regulated in the order of angiogenesis>cell proliferation≈cell growth≈apoptosis≈cell migration>glycolysis (FIG. 12B). Therefore, based on the results, it was revealed that the NDRG3 played an important role in hypoxic responses.

<6-2> Confirmation of Promotion of Angiogenesis by NDRG3 Under Hypoxic Condition

To check an effect of the NDRG3 on the angiogenesis highly relevant to the hypoxia among the functions of the NDRG3 confirmed through the analysis of Example 6-1, a tube forming assay, RT-PCR, Western blotting, and an in vivo angiogenesis assay were performed using the cells in which the NDRG3 expression was inhibited.

Specifically, a tube forming assay was performed to check a change in angiogenesis activity by the NDRG3 deletion. First, the Huh-7 cells (2×10⁵ cells/ml) in which the expression of the NDRG3 was inhibited using shNDRG3 according to the method described in Example 4-4, and the control Huh-7 cells were cultured for 24 hours under a hypoxic condition (1% O₂), and a culture broth (1 ml) was recovered, and then cultured with human umbilical vein endothelial cells (HUVEC; 1×10⁵ cells/ml) for 6 to 12 hours in a 6-well dish previously coated with Matrigel to observe formation of tubes (FIG. 13A).

Also, to check expression of angiogenic markers (IL8, IL1α and IL1β), COX-2, and PAI-1 in response to the NDRG3 deletion under a hypoxic condition, the NDRG3-deleted Huh-7 cells obtained by the method described in Example 6-1, and the control Huh-7 cells were statically cultured for 24 hours under a normoxic condition (21% O₂) or a hypoxic condition (1% O₂), and then collected. Also, HeLa cells were transfected with the NDRG3 (N66D) variant prepared by the method disclosed in Example 4-3, as described in Example 3, statically cultured for 24 hours under a normoxic condition, and then collected. Thereafter, total RNA was extracted from the cells using the method disclosed in Example 4-4, and then subjected to RT-PCR. Also, a portion of each of the cells were subjected to Western blotting using an anti-NDRG3 antibody, as described in Example 3, thereby confirming the expression of the NDRG3 (FIG. 13B).

In addition, a Matrigel plug assay was performed to analyze in vivo angiogenesis in response to the NDRG3 deletion. First, cold Matrigel (BD Biosciences) was mixed with the NDRG3-deleted Huh-7 cells (1×10⁶ cells/ml) obtained by the method disclosed in Example 6-1, and the control Huh-7 cells (1×10⁶ cells/ml). Thereafter, 500 μl of the mixed Matrigel was subcutaneously injected into abdominal regions of 6-week-old female BALB/c mice (Japan SLC). After 7 days, the mice were sacrificed to obtain Matrigel plugs. To quantify hemoglobin, the Matrigel plugs were homogenized in 500 μl of water contained in an ice bucket, centrifuged at 4° C. for 15 minutes at 12,000 rpm, and then washed. Subsequently, only a supernatant was separated, and reacted with a Drabkin's reagent (Sigma) according to the manufacturer's procedure. Then, an optical density of the supernatant was measured at a wavelength of 570 nm using a spectrophotometer, and plotted in a graph (FIG. 13C).

As a result, as shown in FIGS. 13A and 13B, it was confirmed that the tube formation was reduced compared to the control in the case of the cells in which the NDRG3 expression was inhibited under the hypoxic condition, indicating that the NDRG3 deletion inhibited the angiogenesis activity induced through the hypoxic response (FIG. 13A). Also, it was confirmed that the expression of the angiogenic markers (IL8, IL1α and IL1β), COX-2 and PAI-1 mRNA was significantly reduced in the NDRG3-deleted cells kept under the hypoxic condition (Left panel of FIG. 13B), and the mRNA expression of the factors was increased in the cells in which the NDRG3 (N66D) variant in which the PHD2 binding sites were mutated were overexpressed (Right panel of FIG. 13B). Accordingly, it was revealed that the angiogenic factors were increasingly expressed by the NDRG3 in the hypoxic response, resulting in promoted angiogenesis activity (FIGS. 13A and 13B).

Also, as shown in FIG. 13C, it was confirmed that a hemoglobin concentration was reduced in the mice into which the cells in which the NDRG3 expression was inhibited was transplanted, indicating that the angiogenesis was promoted in vivo by the NDRG3, the results of which were comparable to the previous results (FIG. 13C).

<6-3> Confirmation of Promotion of Cell Proliferation by NDRG3 Under Hypoxic Condition

To check an effect of the NDRG3 on the cell proliferation highly relevant to the hypoxia among the functions of the NDRG3 confirmed through the analysis of Example 6-1, an MTT assay was performed to analyze the cell growth and a volume of in vivo xenograft tumor, and an immunofluorescent staining assay, Western blotting and RT-PCR were performed.

Specifically, to check the cell growth in response to the NDRG3 deletion, an MTT assay was performed. First, the NDRG3-deleted Huh-1 cells prepared by the method disclosed in Example 6-1, and the control Huh-1 cells was seeded at a density of 2,000 cells/well in a 96-well plate, and then cultured at 37° C. in a 5% CO₂ incubator under a hypoxic condition (3% O₂) over time. Thereafter, the cells were treated with a 1 mg/ml MTT solution diluted with PBS, and reacted for 2 hours. Then, a medium containing the MTT was removed, and the cells were treated with 100 μl of DMSO to dissolve MTT formazan crystals. Subsequently, an optical density of the cell suspension was measured at a wavelength of 570 nm using a spectrophotometer (FIG. 14A).

Also, to determine a degree of tumorigenesis in response to the NDRG3 and HIF deletion, each of the Huh-7 cells (2×10⁶ cells/100 μl) in which the expression of the NDRG3, HIF-1α, HIF-2α, NDRG3 and HIF-1α or the expression of the NDRG3 and HIF-2α prepared by the methods disclosed in Examples 4-4 and 5-4 was inhibited was subcutaneously administered to the flanks of 6-week-old female BALB/c mice (FIG. 14B). Also, the Huh-1 cells (2×10⁶ cells/100 μl), which had been transfected with the NDRG3 (N66D) variant prepared by the method disclosed in Example 4-3, as described in Example 3, were subcutaneously administered to the flanks of the mice, as described above (FIG. 14C). Thereafter, the Huh-1 cells were imaged to compare the sizes of tumors at 16 and 20 days of administration (FIGS. 14B and 14C).

In the mice in which the Huh-7 cells in which the NDRG3, HIF-1α, HIF-2α, NDRG3 and HIF-1α expression or the NDRG3 and HIF-2α expression was inhibited were transplanted (FIG. 14D), and the mice in which the Huh-1 cells in which the NDRG3 (N66D) variant was overexpressed were transplanted (FIG. 14E), the volumes of tumor were measured on given time points after transplantation using a caliper. The volumes of the tumors were calculated by measuring a length (a), a width (b), and a height (c) according to the following Equation 1, and then plotted in a graph (FIGS. 14D and 14E).

$$Tumor volume = \frac{\begin{matrix}Tumor length ( a ) \times Tumor width ( b ) \times \\ Tumor height ( c )\end{matrix}}{2} \qquad [ Equation 1 ]$$

Also, tumor tissues were extracted from the mice in which the Huh-7 cells×10⁶ cells 100 μl) in which the NDRG3, HIF-1α and HIF-2α expression was inhibited were transplanted, and then fixed in 10% formalin at room temperature for a day. Thereafter, the tumor tissues were embedded in paraffin, and microtomed into sections with a thickness of 4 μm. Then, the microtomed tumor tissues were permeated in 0.3% TritonX-100/PBS at room temperature for 5 minutes, cultured in a blocking solution (PBS supplemented with 1% BSA) for 30 minutes. Subsequently, the tumor tissues were reacted with an anti-ki67 antibody used to determine the presence of a cell proliferation biomarker Ki67, and anti-IL8, anti-CD31 and anti-NDRG3 antibodies used to the presence of angiogenesis biomarkers IL8 and CD31 as the primary antibodies according to the method disclosed in Example 5-1, reacted with a secondary antibody [Alexa Flour 488-conjugated goat anti-rabbit IgG (1/1,000) or Alexa Flour 594-conjugated goat anti-mouse IgG (1/1,000)] and DAPI, and then visualized under a Zeiss LSM 510 confocal microscope (FIG. 14F).

In addition, tumor tissues were extracted from the mice in which the Huh-7 cells (2×10⁶ cells/100 μl) in which the NDRG3, HIF-1α and HIF-2α expression was inhibited were transplanted, and then lyophilized with liquid nitrogen. Thereafter, RT-PCR was performed by the method disclosed in Example 4-2 to determine expression of mRNA, and Western blotting was performed using the anti-NDRG3 and anti-β-actin antibodies according to the method disclosed in Example 2 to determine expression of proteins (FIG. 14G).

As a result, as shown in FIG. 14A, it was confirmed that the cell growth was slowed down over time in the case of the cells in which the NDRG3 was deleted under a light hypoxic condition, compared to the control (FIG. 14A).

Also, as shown in FIGS. 14B to 14E, it was confirmed that the growth of tumor in the mice in which the cells in which the NDRG3 expression was inhibited were transplanted was inhibited over time, compared to the control and the mice in which the cells in which the HIF expression was inhibited were transplanted. In particular, it was confirmed that no tumor was formed in the mice in which the cells in which both of the NDRG3 and HIF-1α or -2α were deleted were transplanted (FIGS. 14B and 14D). On the other hand, it was confirmed that the size of tumor in the mice in which the cells in which the PHD2 docking site-mutated NDRG3 (N66D) variant was overexpressed were transplanted significantly increased, compared to the control. In particular, it was confirmed that no tumor was formed in the control up to 20 days after the transplantation, but the tumor size increased to approximately 900 mm³ in the case of the mice in which the NDRG3 (N66D) variant-overexpressing cells were transplanted, indicating that the tumor growth was promoted by the NDRG3 (N66D) variant (FIGS. 14B to 14E).

In addition, as shown in FIGS. 14F and 14G, it was confirmed that the protein expression of a cell proliferation marker Ki-67 was inhibited in the case of the tumor tissues of the mice in which the NDRG3-deleted cells were transplanted (FIG. 14F). Also, it was confirmed that the protein and mRNA expression of tumor angiogenic markers IL8 and CD31 were inhibited in the tumor tissues of the mice in which the NDRG3-deleted cells were transplanted (FIGS. 14F and 14G). Accordingly, based on the results of Example 6, it was revealed that the NDRG3 played an important role in promoting the angiogenesis and cell proliferation under a sustained hypoxic condition.

### <Example 7> Confirmation of Effect on L-Lactate Generation in NDRG3-Mediated Hypoxic Response

<7-1> Confirmation of NDRG3 Protein Accumulation and Lactate Generation Under Hypoxic Condition

The accumulation and degradation of the NDRG3 protein at long lag periods confirmed in Example 5-2 show that several processes are involved in regulating hypoxic expression of the NDRG3. Therefore, to determine the relevance between the NDRG3 and the biochemical characteristics involved in hypoxia, measurement of lactate generation under a hypoxic condition, Western blotting, and RT-PCR were performed, and an in vitro ubiquitination assay was performed to determine the ubiquitination of the NDRG3.

Specifically, MCF-7 cells were statically cultured for 24 hours under a normoxic condition (21% O₂), as described in Example 5-2, or statically cultured by time under a hypoxic condition (1% O₂), and then collected and lysed, as described in Example 3. Thereafter, the cell lysate was subjected to Western blotting using anti-NDRG3, anti-HIF-1α and anti-β-actin antibodies, and L-lactate generation was determined according to the manufacturer's procedure using an EnzyChrom™ L-lactate assay kit (BioAssay Systems), and then plotted in a graph. The values were normalized to an L-lactate standard curve (FIG. 15A).

Also, to check an effect of inhibition of the L-lactate generation on expression of the NDRG3 protein, MCF-7 cells were treated with a varying concentration of sodium oxamate that was a lactate dehydrogenase A (LDHA) inhibitor, statically cultured for 24 hours under a hypoxic condition (1% O₂), as described in Example 5-2, and then collected and lysed, as described in Example 3. Thereafter, the cell lysate was subjected to Western blotting using anti-NDRG3, anti-HIF-1α and anti-β-actin antibodies, as described above, and L-lactate generation was determined, and then plotted in a graph. The values were normalized to the L-lactate standard curve (FIG. 15B).

In addition, to check an effect of an amount of L-lactate generation on expression of the NDRG3 protein, MCF-7 cells in which a monocarboxylate transporter MCT4 involved in LDHA or lactate export was deleted were prepared using siLDHA (siGENOME SMARTpool, Dharmacon) or siMCT4 (siGENOME SMARTpool, Dharmacon) according to the method disclosed in Example 4-2. Thereafter, the MCF-7 cells were statically cultured for 24 hours under a hypoxic condition (1% O₂), as described in Example 5-2, and then collected and lysed, as described in Example 3. Then, the MCF-7 cells were subjected to Western blotting using anti-NDRG3, anti-HIF-1α and anti-β-actin antibodies, as described above, and L-lactate generation was then determined, and plotted in a graph (FIG. 15C). Also, RT-PCR was performed as described in Example 4-2 to confirm the LDHA and MCT4 deletion (FIG. 15C).

Additionally, to check an effect of inhibition of glycolysis on expression of the NDRG3 protein, MCF-7 cells were treated with a varying concentration of 2-deoxyglucose (2-DG) inhibiting the glycolysis, statically cultured for 24 hours under a hypoxic condition (1% O₂), as described in Example 5-2, and then collected and lysed, as described in Example 3. Thereafter, the MCF-7 cells were subjected to Western blotting using anti-NDRG3 and anti-β-actin antibodies (FIG. 15D).

Also, to determine expression of the NDRG3 protein caused by the excessive lactate generation, a Flag-tagged LDHA expression vector was constructed, and HeLa cells were then transfected with the expression vector, as described in Example 3, and statically cultured for 24 hours under a normoxic condition (21% O₂). Thereafter, the HeLa cells were further treated with 50 mM pyruvate, statically cultured for 24 hours under a light hypoxic condition (3% O₂), collected, and then lysed. The cell lysate was subjected to Western blotting using anti-NDRG3, anti-Flag and anti-β-actin antibodies (FIG. 15E).

Further, to check an effect of lactate generation on ubiquitination of the NDRG3, an in vitro ubiquitination assay was performed. First, HEK293T cells were transfected with the Flag-tagged PHD2 and HA-tagged VHL variants, as described in Example 3, collected, and then lysed. Thereafter, the protein lysate (500 μg) was reacted with an anti-HA affinity gel (Sigma) or an anti-Flag affinity gel (Sigma) at 4° C. for a day, and centrifuged to obtain recombinant PHD2 proteins or VHL proteins. Then, the recombinant PHD2-/VHL-binding proteins were untreated or treated with L-lactate (pH 7.0, 20 mM), and then reacted at 37° C. for an hour in 50 μl of a solution including recombinant human NDRG3-GST (4 μg) prepared by the method described in Example 4-1, 500 ng of a ubiquitin-activating enzyme (E1, Upstate), 1 μg of a ubiquitin-conjugating enzyme (E2 (UbcH5a), Upstate), 2.5 μg of ubiquitin-Flag (Sigma), 20 mM HEPES (pH 7.3), 5 mM MgCl₂, 1 mM DTT, and 2 mM ATP. Subsequently, the mixture was incubated with a GST-conjugated agarose resin at 4° C. for 4 hours, and the precipitate was washed, and subjected to Western blotting using an anti-Flag antibody, as described in Example 3 (FIG. 15F).

As a result, as shown in FIGS. 15A and 15B, it was confirmed that the expression of the HIF-1α protein significantly increased and then decreased at the beginning of hypoxic condition, but the lactate generation and the accumulation of the NDRG3 protein significantly increased as the hypoxic condition lasted (FIG. 15A). On the other hand, it was confirmed that, when the cells were treated with sodium oxamate to inhibit the lactate generation, the accumulation of the NDRG3 protein was inhibited in proportion to the lactate expression (FIG. 15B), indicating that the expression of the NDRG3 protein under the hypoxic condition was involved in the lactate generation (FIGS. 15A and 15B).

Also, as shown in FIGS. 15C to 15E, it was confirmed that the expression of the NDRG3 protein under the hypoxic condition was reduced when the lactate generation was inhibited by inhibiting the glycolysis through LDHA deletion or 2-deoxyglucose treatment, but the accumulation of the NDRG3 protein under the hypoxic condition increased when lactate increased through the MCT4 deletion, or LDHA overexpression and/or supply of a pyruvate-containing medium. Accordingly, it was revealed that the lack of oxygen and production of glycolytic lactate are further required for accumulation of the NDRG3 protein, unlike the hypoxic induction of the HIF protein (FIGS. 15C to 15E).

Also, as shown in FIG. 15F, it was confirmed that the ubiquitination of the recombinant NDRG3 protein by a PHD2/VHL complex was reduced when the recombinant NDRG3 protein was treated with lactate, indicating that the ubiquitination of the NDRG3 protein was inhibited due to the lactate generation under the hypoxic condition, and the NDRG3 protein was accumulated in the cells (FIG. 15F).

<7-2> Confirmation of Binding of NDRG3 and Lactate Under Hypoxic Condition

To confirm interaction between lactate and NDRG3 protein under oxygen conditions, a recombinant NDRG3 protein was prepared, and a recombinant NDRG3 variant protein was prepared using a site-directed mutation. Then, the proteins were subjected to an in vitro binding assay, immunostaining and Western blotting.

Specifically, to confirm interaction between L-lactate and the NDRG3 protein, a site-directed mutation was performed, as described in Example 4-3, using the pGEX-4T-2-NDRG3, which was cloned by the method disclosed in Example 4-1, as a template, and primers listed in the following Table 4, thereby obtaining a pGEX-4T-2-NDRG3 (G138W) variant construct in which glycine (G) that was an amino acid at position 138 among the L-lactate binding sites of the NDRG3 was substituted with glycine (G). Thereafter, to express the prepared recombinant protein, E. coli strain BL21 was transformed with the recombinant wild-type pGEX-4T-2-NDRG3 and pGEX-4T-2-NDRG3 (G138W) plasmid vectors, and the recombinant protein was purified using a GST-conjugated agarose resin, as described in Example 2. Then, the recombinant protein was electrophoresed on 9% SDS-PAGE, as described in Example 3, and then stained with Coomassie Brilliant Blue (CBB) to verify the detection. To determine expression of the recombinant protein, Western blotting was performed using anti-GST and anti-NDRG3 antibodies (FIG. 16A).

TABLE 4

| Primer | | Sequence (5′→3′) |
|---|---|---|
| NDRG3 | Sense | GTTTGGGCTGGAGCTTACATCCTCAGC |
| (G138W) | | (SEQ ID NO: 25) |
| | Antisense | TCCAATTCCAATGATGCTTTTCAGGCT |
| | | (SEQ ID NO: 26) |

Also, to confirm interaction between L-lactate and the NDRG3 protein, an in vitro binding assay was performed. GST or the recombinant GST-NDRG3 protein (50 μg) was reacted with 0.5 μCi of unlabeled L-lactate (pH 7.0, Sigma) and L-[¹⁴C]-lactate (PerkinElmer) at 30° C. for an hour. Thereafter, a GST-conjugated agarose resin was added thereto, and the resulting mixture was reacted for 4 hours. Then, a resin to which the NDRG3-GST protein was bound was separated from the reaction mixture, and impurities were removed using PBS. Subsequently, the NDRG3-bound agarose resin was transferred to a scintillation solution containing 2 ml of an LSC-cocktail (PerkinElmer), and ¹⁴C values were measured, and plotted in a graph (Left panel of FIG. 16B). Also, GST, the recombinant GST-NDRG3 protein (50 μg) or the recombinant GST-NDRG3 (G138W) variant protein (50 μg) was reacted with 0.5 μCi of L-[¹⁴C]-lactate (PerkinElmer) at 30° C. for an hour, and ¹⁴C values were then measured in the same manner as described above, and plotted in a graph (Right panel of FIG. 16B).

In addition, NDRG3 variants were prepared to confirm interaction between L-lactate and the binding sites of the NDRG3 protein under a hypoxic condition. First, a site-directed mutation was performed, as described in Example 4-3, using the Myc-tagged NDRG3 expression vector as a template, thereby obtaining a Myc-NDRG3 (D62L) variant in which aspartic acid (Asp, D) as an amino acid at position among the L-lactate binding sites of NDRG3 was substituted with leucine (Leu, L), a Myc-NDRG3 (G138W) variant in which glycine (G) as an amino acid at position 138 among the L-lactate binding sites of NDRG3 was substituted with arginine (Arg, R), a Myc-NDRG3(A139E) variant in which alanine (Ala, A) as an amino acid at position 139 among the L-lactate binding sites of NDRG3 was substituted with glutamic acid (Glu, E), and a Myc-NDRG3(Y229P) variant in which tyrosine (Tyr, Y) as an amino acid at position 229 among the L-lactate binding sites of NDRG3 was substituted with proline (Pro, P). Therefore, HEK293T cells were transfected with each of the Myc-NDRG3 and Myc-NDRG3 variants, as described in Example 3, and then treated with 20 μM MG132 under a normoxic condition (21% O₂) or a hypoxic condition (1% O₂), as described in Example 5-2. Then, the HEK293T cells were statically cultured for 24 hours under a hypoxic condition (1% O₂), collected, and then lysed. Subsequently, the cell lysate was subjected to Western blotting using anti-Myc and anti-β-actin antibodies (FIG. 16C).

Also, to determine expression of the NDRG3 protein, when the hypoxic condition returned to the normoxic condition, MCF-7 cells were statically cultured for 24 hours under a hypoxic condition (1% O₂), the medium was replaced with a fresh medium. Thereafter, the MCF-7 cells were further statically cultured by time under a normoxic condition (21% O₂), and then collected and lysed, as described in Example 3. Then, the cell lysate was subjected to Western blotting using anti-NDRG3, anti-HIF-1α and anti-β-actin antibodies (FIG. 16D).

As a result, as shown in FIG. 16A to 16C, it was confirmed that the recombinant NDRG3 (G138W) variant protein in which the NDRG3 and the lactate binding sites of the NDRG3 were mutated was expressed (FIG. 16A), and that the in vitro binding of the recombinant NDRG3 protein to lactate increased (Left panel of FIG. 16B), and the NDRG3 variant protein had a reduced lactate-binding affinity (Right panel of FIG. 16B). Also, it was confirmed that the protein accumulation increased under the hypoxic condition in the case of the normal NDRG3 protein, and the protein accumulation decreased under the hypoxic condition in the case of all the variants in which the lactate binding sites of NDRG3 were mutated (FIG. 16C), and that the NDRG3 bound to lactate under the hypoxic condition, indicating that the binding of the NDRG3 to lactate was involved in the accumulation of the NDRG3 protein (FIGS. 16A to 16C).

Also, as shown in FIG. 16D, it was confirmed that the HIF-1α protein was rapidly removed when the HIF-1α protein was reoxygenated, but the NDRG3 protein accumulated to form an NDRG3 lactate complex under the hypoxic condition was stably maintained in the cells even when the hypoxic condition returned to the normoxic condition (FIG. 16D). Accordingly, based on the results of Example 7, it was revealed that hypoxia-induced lactate served as a sensor for NDRG3 in a sustained hypoxic response to promote a HIF-independent biological reaction by the NDRG3.

### <Example 8> Confirmation of Functions of Lactate-Dependent NDRG3 in Hypoxic Response

<8-1> Confirmation of Promotion of Lactate-Dependent Cell Growth by NDRG3 in Hypoxic Response

To check an effect of NDRG3 on lactate-dependent cell growth under a hypoxic condition, an MTT assay, a colony forming assay, and in vivo xenograft tumor volumetry were performed using the NDRG3 variants, and the cells in which the lactate generation was inhibited.

Specifically, to check an effect of the ectopic NDRG3 (N66D) variant on the cell growth after inhibition of the lactate generation, Huh-1 cells were transfected with an ectopic variant NDRG3 (N66D) expression vector, as described in Example 4-3. Then, the Huh-1 cells and the NDRG3 (N66D) variant-overexpressing Huh-1 cells were seeded at a density of 1,000 cells/well in a 96-well plate, treated with a varying concentration of sodium oxamate, statically cultured by time under a light hypoxic condition (3% O₂). Thereafter, to check the cell growth, an MTT assay was performed, as described in Example 6-3, and plotted in a graph (FIG. 17A).

Also, to check an effect of the ectopic NDRG3 (N66D) variant on the cell growth after inhibition of the lactate generation, a colony forming assay was performed. The Huh-1 cells and the NDRG3 (N66D) variant-overexpressing Huh-1 cells were seeded at a density of 0.5×10⁴ cells/2 ml in a 6-well plate, and untreated or treated with 40 mM sodium oxamate. Then, both the cells were cultured for 10 days under a normoxic condition (21% O₂) while replacing the medium with a fresh medium every 3 days. Colonies formed after 10 days of the culture were confirmed, fixed in 100% methyl alcohol, and then stained with a 30% Giemsa stain solution (Sigma) to be counted (FIG. 17B).

In addition, to check an effect of the ectopic NDRG3 (N66D) variant on the cell growth after inhibition of the lactate generation caused by the LDHA deletion under a hypoxic condition, an MTT assay was performed. First, Huh-1 cells in which LDHA was deleted were prepared by the method disclosed in Example 4-4, and then transfected with an NDRG3 (N66D) variant expression vector, as described in Example 3, thereby obtaining LDHA-deleted and NDRG3 (N66D) variant-overexpressing Huh-1 cells. Thereafter, the control GFP-deleted Huh-1 cells, the LDHA-deleted Huh-1 cells and the LDHA-deleted/NDRG3 (N66D)-overexpressing Huh-1 cells were seeded at a density of 1,000 cells/well, cultured in a 96-well plate, statically cultured by time under a light hypoxic condition (3% O₂). Then, to check the cell growth, an MTT assay was performed, as described in Example 6-3, and plotted in a graph (FIG. 17C).

Additionally, to check an effect of the ectopic NDRG3 (N66D) variant on the cell growth after in vivo inhibition of the lactate generation caused by the LDHA deletion, tumor cells were transplant into mice, and the volume of tumor was then measured. The control GFP-deleted Huh-1 cells, the LDHA-deleted Huh-1 cells, and the LDHA-deleted/NDRG3 (N66D)-overexpressing Huh-1 cells were transplanted into mice according to the method disclosed in Example 6-3, and the volumes of tumor were then measured on given time points using a caliper, and then plotted in a graph (FIG. 17D).

Also, to check an effect of the inhibition of the lactate generation on expression of the ectopic variant NDRG3 (N66D) protein under a sustained hypoxic condition, Huh-1 cells and the NDRG3 (N66D) variant-overexpressing Huh-1 cells were untreated or treated with 40 mM sodium oxamate, statically cultured by time under a light hypoxic condition, and then collected and lysed, as described in Example 3. Thereafter, the cell lysate was subjected to Western blotting using anti-NDRG3 and anti-β-actin antibodies (FIG. 17E).

Further, to check an effect of the ectopic NDRG3 (N66D) variant on the lactate generation under a sustained hypoxic condition, the Huh-1 cells and the NDRG3 (N66D) variant-overexpressing Huh-1 cells, which had not been treated or treated with sodium oxamate and statically cultured by time under the light hypoxic condition as described above, were collected, and L-lactate generation was determined according to the method disclosed in Example 7-1, and then plotted in a graph. The values were normalized to an L-lactate standard curve (FIG. 17F).

As a result, as shown in FIGS. 17A to 17D, it was confirmed that the cell growth was inhibited in a concentration-dependent manner when the cells were treated with sodium oxamate under the hypoxic condition, but the cell growth increased even when the cells were treated with sodium oxamate under the hypoxic condition after the ectopic NDRG3 variant was overexpressed (FIG. 17A). It was confirmed that, when the ectopic NDRG3 variant was overexpressed, the tumor growth was promoted like those under the hypoxic condition even when the lactate generation was inhibited under the normoxic condition (FIG. 17B). Also, it was confirmed that the tumor growth was inhibited in vitro and in vivo when the lactate generation was inhibited through the LDHA deletion, but the tumor growth significantly increased when the ectopic NDRG3 variant was overexpressed although LDHA was deleted (FIGS. 17C and 17D).

Also, as shown in FIGS. 17E and 17F, it was confirmed that the accumulation of NDRG3 was inhibited when the cells were treated with sodium oxamate under the hypoxic condition to inhibit the lactate generation, but, when the ectopic NDRG3 variant was overexpressed, the accumulation of the NDRG3 variant lasted although the lactate generation was inhibited (FIG. 17E). Also, it was revealed that, when the cells were untreated and treated with sodium oxamate under the hypoxic condition, there was no change in the lactate generation even when the ectopic NDRG3 variant was overexpression, indicating that the ectopic NDRG3 variant had no direct effect on lactate production (FIG. 17F). Accordingly, based on the results, it was revealed that the NDRG3 was an important mediator for lactate-inducible cell growth under the sustained hypoxic condition.

<8-2> Confirmation of Promotion of Lactate-Dependent Angiogenesis by NDRG3 in Hypoxic Response

To check an effect on the NDRG3 on lactate-dependent angiogenesis under a hypoxic condition, a tube forming assay was performed using the ectopic NDRG3 variant-overexpressing cells.

Specifically, Huh-1 cells in which the NDRG3 (N66D) variant was overexpressed were prepared, and the control cells and the NDRG3 (N66D) variant-overexpressing Huh-1 cells were untreated or treated with sodium oxamate, statically cultured for 24 hours under a hypoxic condition (1% O₂). Thereafter, the cells were collected, as described in Example 6-2, and then subjected to a tube forming assay (FIG. 18).

As a result, as shown in FIG. 18, it was confirmed that the angiogenesis was inhibited under the hypoxic condition when the cells were treated with sodium oxamate to inhibit the lactate generation, but, when the ectopic NDRG3 variant was overexpressed, the angiogenesis increased again under the hypoxic condition even when the lactate generation was inhibited, indicating that the NDRG3 was an important mediator for lactate-inducible angiogenesis under the sustained hypoxic condition (FIG. 18). Accordingly, based on the results of Example 8, it was revealed that the lactate served as an important signal for hypoxic cell proliferation and angiogenesis, and the NDRG3 served as an important mediator for lactate-inducible cell proliferation and angiogenesis under the sustained hypoxic condition.

### <Example 9> Confirmation of Molecular Regulation of NDRG3-Mediated Hypoxic Response

<9-1> Confirmation of Regulation of c-Raf-ERK Activity by NDRG3 Under Hypoxic Condition

To check a role of the NDRG3 in a lactate-inducible molecular response under a hypoxic condition, an in vitro kinase assay, a pull-down assay, an immunoprecipitation assay, and Western blotting were performed.

Specifically, GFP- or NDRG3-deleted PLC/PRF/5 cells were prepared using the control shGFP or shNDRG3 according to the method disclosed in Example 4-4, statically cultured for 24 hours under a hypoxic condition (1% O₂), and then collected, as described in Example 3. Thereafter, phosphorylated proteins were identified according to the manufacturer's procedure using a Human Phospho-Kinase Array kit (FIG. 19A).

Also, to check a molecular regulatory function of the NDRG3 in response to an expression level of the NDRG3, Huh-1, Huh-7 and 786-O cells were statically cultured for 24 hours under a normoxic condition (21% O₂), collected and lysed, as described in Example 3, and then subjected to Western blotting using anti-NDRG3, anti-phosphorylated ERK1/2, anti-ERK1/2 and anti-β-actin antibodies (FIG. 19B).

In addition, to examine a molecular function of the NDRG3 under a hypoxic condition, GFP- or NDRG3-deleted SK-HEP-1 cells were prepared using the control shGFP or shNDRG3 according to the method described in Example 4-4, statically cultured by time under a hypoxic condition, collected and lysed, as described in Example 3, and then subjected to Western blotting using anti-phosphorylated ERK1/2 and anti-ERK1/2 antibodies (Upper panel of FIG. 19C). Also, the prepared cells were statically cultured for 24 hours under a hypoxic condition, collected and lysed, as described in Example 3, and then subjected to Western blotting using anti-NDRG3, anti-phosphorylated c-Raf (S338), anti-c-Raf, anti-phosphorylated B-RAF1 (S445), anti-B-RAF1, anti-phosphorylated A-RAF (S299), anti-A-RAF, and anti-β-actin antibodies (Lower panel of FIG. 19C).

Additionally, to confirm in vitro interaction between the NDRG3 and c-Raf in response of a hypoxic response, first, a recombinant plasmid pET-28a-c-Raf construct coding for c-Raf was cloned. Thereafter, a BL21 E. coli strain was transfected with the c-Raf expression vector, as described in Example 7-1, to obtain a recombinant protein. Then, the c-Raf recombinant protein was purified using a Ni-NTA agarose resin according to the manufacturer's procedure. Subsequently, the purified recombinant c-Raf-His protein and NDRG3-GST were reacted with a Ni-NTA agarose resin, as described in Example 4-1, and then subjected to a His pull-down assay. Then, the recombinant protein was subjected to a Western blotting using an anti-NDRG3 antibody, as described in Example 3 (Left panel of FIG. 19D). Also, after a Flag-tagged c-Raf expression vector was cloned, HeLa cells were transfected with the Flag-tagged c-Raf vector, as described in Example 3. Then, the Flag-c-Raf-overexpressed HeLa cells were statically cultured for 24 hours under a hypoxic condition (1% O₂), immunoprecipitated with anti-FLAG M2 beads, and then subjected to Western blotting using an anti-NDRG3 antibody (Right panel of FIG. 19D).

Also, to examine a molecular function of the NDRG3 under a hypoxic condition, SK-HEP-1 cells in which the NDRG3 expression was inhibited were prepared using the control shGFP or shNDRG3 according to the method described in Example 4-4, and transfected with a Flag-tagged c-Raf expression vector, as described in Example 3, to obtain NDRG3-deleted/c-Raf-overexpressing cells. Also, the cells were transfected with Myc-tagged NDRG3 (N66D) and Flag-tagged c-Raf expression vectors, as described in Example 3, to obtain NDRG3 (N66D) and c-Raf-overexpressing HEK293T cells. Thereafter, the cells were statically cultured for 24 hours under a normoxic condition (21% O₂), collected and lysed, as described in Example 3, and then subjected to Western blotting using anti-NDRG3, anti-phosphorylated c-Raf (S338), anti-c-Raf, anti-phosphorylated B-RAF1 (S445), anti-B-RAF1, anti-phosphorylated ERK1/2, anti-ERK1/2, and anti-β-actin antibodies (FIG. 19E).

In addition, to determine phosphorylation by NDRG3 under a hypoxic condition, an in vitro kinase assay was performed using a [γ-³²P]-ATP label (PerkinElmer). First, HEK293T cells transfected with a Myc-tagged NDRG3 (N66D) expression vector were prepared, as described in Example 3, and an NDRG3 protein was immunoprecipitated with an anti-Myc affinity gel. Thereafter, the immunoprecipitated NDRG3 complex was tagged with a radioactive label, and untreated or treated with LY333531 (5 μM) serving as a PKC inhibitor. Then, the resulting mixture was reacted 30° C. for an hour in 40 μl of a reaction buffer [a PKC activation buffer and a PKC co-activation buffer contained in a SignaTECT protein kinase C analysis kit (Promega), 10 μCi of [γ-³²P]-ATP, and 2 μg of a purified c-Raf-GST recombinant protein]. Then, the reaction mixture was electrophoresed on 8% SDS-PAGE, and ³²P-labeled c-Raf was detected using autoradiography (FIG. 19F).

Further, to determine lactate dependence of activation of an NDRG3-mediated molecular pathway under a hypoxic condition, MCF-7 cells were statically cultured for 24 hours under a normal condition (21% O₂), and the MCF-7 cells in which LDHA expression was inhibited, as prepared by the method described in Example 7-1, and cells untreated or treated with sodium oxamate were statically cultured 24 hours under a hypoxic condition (1% O₂). Thereafter, the cells were collected and lysed, as described in Example 3, and then subjected to Western blotting using anti-NDRG3, anti-phosphorylated c-Raf (S338), anti-c-Raf, anti-phosphorylated ERK1/2, anti-ERK1/2, and anti-β-actin antibodies (FIG. 19G).

As a result, as shown in FIGS. 19A to 19D, it was confirmed that the phosphorylation of ERK1/2 under a sustained hypoxic condition was reduced when the NDRG3 was deleted (FIG. 19A, and upper panel of FIG. 19C), and phosphorylation levels of ERK1/2 differed in proportion to an expression level of the NDRG3 protein in various types of cells in which the NDRG3 protein was expressed at different levels under a normoxic condition (FIG. 19B), indicating that the NDRG3 activated ERK1/2 in a sustained hypoxic response. Also, it was confirmed that, when the NDRG3 was deleted, the phosphorylation of c-Raf and B-RAF1 as well as the ERK1/2 phosphorylation was inhibited as the hypoxic condition lasted (Lower panel of FIG. 19C), and the NDRG3 interacted with c-Raf in an in vitro hypoxic response (Left panel of FIG. 19D) and in an intracellular hypoxic response (Right panel of FIG. 19D). Accordingly, it was revealed that the NDRG3 was involved in activation of ERK and c-Raf under the hypoxic condition (FIGS. 19A to 19D).

Also, as shown in FIGS. 19E and 19F, it was confirmed that, when the ectopic NDRG3 (N66D) variant was overexpressed, the phosphorylation of ERK1/2 and c-Raf was induced even under a normoxic condition (FIG. 19E), and a molecular complex of the ectopic NDRG3 variant also mediated phosphorylation of the recombinant c-Raf protein in vitro (FIG. 19F).

In addition, as shown in FIG. 19G, it was confirmed that, when the lactate generation under hypoxic condition was inhibited, the expression of the NDRG3 protein was also inhibited, and activation of the c-Raf-ERK pathway was inhibited (FIG. 19G). Accordingly, based on the results, it was revealed that the NDRG3 served as an essential mediator for activation of the c-Raf-ERK signal induced by lactate in a hypoxic response.

<9-2> Confirmation of Regulation of Kinase Pathway by NDRG3 Under Hypoxic Condition

RACK1 is a scaffold protein for PKC, and is well known to maintain active conformation (Lendahl, U. et al., Nat. Rev. Genet., 2009(10), 821-832), and PKC is reported to activate c-Raf through phosphorylation (Epstein, A. C. et al., Cell, 2001(107), 43-54, Mahon, P. C. et al., Genes Dev, 2001(15), 2675-2686). Therefore, an immunoprecipitation assay, and Western blotting were performed to determine the relevance of RACK1 in activation of the kinase pathway by NDRG3 under a hypoxic condition, and protein structure modeling was performed to confirm formation of complexes between the NDRG3 and proteins involved in the kinase pathway.

Specifically, to check interaction between the NDRG3 and RACK1, HeLa cells were transfected with NDRG3 and/or Flag-tagged RACK1 expression vectors, as described in Example 3, treated with 20 μM MG132 for 8 hours under a normoxic condition (21% O₂), cultured, collected, lysed, and then immunoprecipitated with anti-FLAG M2 beads. Thereafter, to check the presence of the NDRG3 bound to RACK-1, Western blotting was performed using an anti-NDRG3 antibody (FIG. 20A).

Also, to verify the relationship between the proteins involved in the kinase pathway via the NDRG3, C-Raf, RACK1-overexpressing and/or NDRG3-deleted HEK293T cells, and c-Raf, RACK1 and/or NDRG3 (N66D)-overexpressing HEK293T cells were untreated or treated with 5 μM PKC-I (LY333531), as described in Example 9-1, statically cultured for 24 hours under a normoxic condition, collected and lysed, as described in Example 3, and then subjected to Western blotting using anti-NDRG3, anti-phosphorylated c-Raf (S338), anti-c-Raf, anti-phosphorylated ERK1/2, anti-ERK1/2, anti-RACK1, and anti-β-actin antibodies (FIG. 20B).

In addition, to verify the relationship between the proteins involved in the kinase pathway via the NDRG3 under a hypoxic condition, NDRG3 (N66D)-overexpressing and/or RACK1-deleted HeLa cells were prepared using a Myc-NDRG3 (N66D) expression vector and/or siRACK1 (siGENOME SMARTpool, Dharmacon), statically cultured for 24 hours under a hypoxic condition (1% O₂), immunoprecipitated with an anti-Myc affinity gel, as described in Example 3, and then subjected to Western blotting using an anti-PKC-β antibody (FIG. 20C).

Additionally, to check an effect of PKC on ERK activation under a hypoxic condition, HeLa cells were treated under a normoxic condition (21% O₂), or treated with 1 μM PKC-I (GO; GO 6976) or 5 μM PKC-I (LY; LY333531) served as a PKC inhibitor, statically cultured for 24 hours under a hypoxic condition (1% O₂), collected and lysed, as described in Example 3, and then subjected to Western blotting using anti-phosphorylated ERK1/2 and anti-ERK1/2 antibodies (FIG. 20D).

Also, to confirm formation of a NDRG3-cRaf-RACK1-PKC-β complex under a hypoxic condition, HEK293T cells were transfected with Flag-c-Raf and Flag-RACK1 expression vectors, and/or a Myc-NDRG3 (N66D) expression vector, as described in Example 3, statically cultured for 24 hours under a normoxic condition, collected, and then lysed. Therefore, the cells were immunoprecipitated with an anti-Myc affinity gel, electrophoresed on SDS-PAGE, and then subjected to Western blotting using anti-Flag, anti-Myc and anti-PKC-β antibodies (FIG. 20E).

Further, to confirm formation of an NDRG3-cRaf-RACK1-PKC-β complex under a hypoxic condition, a protein docking simulation was performed, as described in Example 4-3. Docking for multiple target structures (that is, NDRG3, RAF1, RACK1 (GNB2L1) and PKC-β) was performed using a two-step experiment. In the first step, the NDRG3 was used as a receptor protein in docking experiments for respective targets. In the second step, a protein-protein interaction product was used as a receptor protein for docking other proteins. The input order for the second experiment was sequentially used for c-Raf, RACK1 and PKC-β. Docking calculation was performed with a root-mean-square deviation (RMSD), and the results were filtered at a level at which the calculated values from a single input matched the calculated values from multiple inputs. Among the filtered result values, the most stable one was selected using the total HEX6.3 score (the sum of shape scores and electrostatistics cores) (Upper panel of FIG. 20F). Also, a series of processes were performed for protein structure modeling, as follows. In the second step, protein-protein BLAST (BLASTp) searches were performed to screen template candidates used for homogeny modeling from structures well kwon in protein data bank (PDB) sequence database, one template candidate having the highest identity score was chosen from the template candidates having a p-value smaller than 10⁻³. In the second step, an alignment process was performed to perform BLAST, and restrained spaces for the structures were checked. In the third step, prediction of protein structures was performed using a well-known homogeny modeling program, that is, Modeller 9v10 (N. Eswar, M. A. et al., Current Protocols in Bioinformatics, John Wiley & Sons, Inc., Supplement 15, 5.6.1-5.6.30, 2006). Finally, the best protein model was selected based on a discrete optimized protein energy (DOPE) evaluation method (Shen M Y, et al., Protein Sci. 2006 November; 15(11): 2507-24) (Lower panel of FIG. 20F).

As a result, as shown in FIGS. 20A to 20D, it was confirmed that the RACK1 interacted with the NDRG3 (FIG. 20A). Also, it was confirmed that, when the NDRG3 was deleted, the c-Raf and ERK1/2 phosphorylation was inhibited even when the RACK1 was overexpressed, but the c-Raf and ERK1/2 phosphorylation increased when the NDRG3 (N66D) variant was overexpressed, and, when the activity of PKC was inhibited, the c-Raf and ERK1/2 phosphorylation was inhibited even when the RACK1 and NDRG3 (N66D) proteins were overexpressed (FIG. 20B). In particular, it was confirmed that the interaction between the NDRG3 and PKC was inhibited under a hypoxic condition when the RACK1 was deleted (FIG. 20C), and the ERK1/2 phosphorylation was inhibited under a hypoxic condition when the activity of PKC was inhibited by a PKC inhibitor (FIG. 20D). Accordingly, it was revealed that the NDRG3 interacted with RACK1 and PKC-β under a hypoxic condition to promote the c-Raf-ERK phosphorylation (FIGS. 20A to 20D).

Also, as shown in FIGS. 20E and 20F, it was confirmed that the NDRG3 existed in the cells in a state in which the NDRG3 formed a complex with c-Raf, RACK1 and PKC-β (FIG. 20E), and a quaternary NDRG3-c-Raf-RACK1-PKC-β complex was flexibly formed through the docking simulation and protein structure modeling (FIG. 20F). Accordingly, based on the results of Example 10, it was revealed that the NDRG3 interacted with RACK1, and c-Raf was phosphorylated by PKC-β induced to the NDRG3-RACK1 complex through such interaction, indicating that the NDRG3 regulated by lactate was a scaffold protein for c-Raf and RACK1.

### <Example 10> Determination of Pathologic Change in Response to NDRG3 Expression

<10-1> Confirmation of Promotion of Tumorigenesis and Angiogenesis in NDRG3-Overexpressing Transgenic Mouse

To check a pathologic effect of expression of the NDRG3, immunohistochemical analysis was performed using NDRG3-overexpressing transgenic mice, to confirm tumorigenesis, Western blotting was performed to confirm expression of the NDRG3 and expression of the activated ERK1/2 protein, and RT-PCR was performed to check a change in gene expression.

Specifically, to check an effect of NDRG3 overexpression on tumorigenesis, tumor formation was checked on given time points until 24 months in NDRG3-overexpressing C57/BL6 transgenic mice (No.: 40) prepared by the method disclosed in Example 1 to homogenously overexpress an NDRG3 gene, and the control mice (No.: 32), and the results of the tumor formation were plotted in graph using a tumor freeKaplan Meier assay (FIG. 21A).

Also, to check an effect of the NDRG3 overexpression on tumorigenesis in various tissues, lungs from the NDRG3-overexpressing transgenic mice and the control mouse, which were 18 months old, intestines from the NDRG3-overexpressing transgenic mice and the control mouse, which were 20 months old, and hypogastric tissues from the NDRG3-overexpressing transgenic mice and the control mouse, which were 9 months old, were extracted, and observed for tumorigenesis (FIG. 21B).

In addition, to check an effect of the NDRG3 overexpression on lymphoma expression, an immunohistological assay was performed using mesenteric lymph nodes, spleens and liver tissues from the NDRG3-overexpressing transgenic mice and the control mouse. First, lymph nodes, spleens and liver tissues were extracted from the NDRG3-overexpressing transgenic mice and the control mouse, and then fixed in 10% formalin at room temperature for a day. Thereafter, the fixed tissues were embedded in paraffin, and microtomed into sections with a thickness of 4 μm. Then, the microtomed tumor tissues were transferred onto silanylated slides (Histoserv). For antigen detection, the microtomed tissue-containing slides were treated with a 0.01 M citrate buffer (pH6.0), and heated at 100° C. for 2 minutes. Subsequently, the slides were cooled down, treated with 3% hydrogen peroxide/PBS for 5 minutes to inactivate intracellular peroxidases in the tissues, and then blocked with 10% non-immune mouse or goat sera for 30 minutes. Then, to detect a B cell marker, CD45R, and a T cell marker, CD3, both of which were expressed in lymphoma, the tissues were labeled with anti-CD45R and anti-CD3 antibodies. Here, the label was detected by an avidin-biotin complex (ABC) method using a 3,3′-diaminobenzidine (DAB) substrate/chromogen solution, and observed and imaged under a microscope. Also, the slides were counterstained with Hematoxylin-Eosin (H&E), and then observed and imaged under the microscope (FIG. 21C).

Additionally, to check an effect of the NDRG3 overexpression on hepatoma, liver tissues were extracted from each of three types of NDRG3-overexpressing transgenic mice TG-2, TG-8 and TG-13 prepared by the method described in Example 1, and then fixed and microtomed, as described above. Thereafter, the microtomed liver tissues were subjected to immunohistological staining, and visualized, as described above. To detect cell proliferation markers, PCNA and Ki-67, a hepatocellular carcinoma (HCC) marker, glutamine synthetase (GS), and heat shock protein 70 (HSP 70), the tissues were labeled with anti-PCNA, anti-HSP70, anti-Ki-67 and anti-GS antibodies. Also, the microtomed tissues were subjected to immunohistological staining using an anti-NDRG3 antibody according to the method described in Example 4-1, and visualized using a confocal microscope (FIG. 21D).

Further, to check an effect of the NDRG3 overexpression on hepatoma at a molecular level, liver tissues were extracted from the NDRG3-overexpressing transgenic mice and the control mouse, and then lyophilized with liquid nitrogen. Therefore, RT-PCR was performed by the method described in Example 4-2. Also, the tissues were lysed, as described in Example 2, and then subjected to Western blotting using anti-NDRG3, anti-phosphorylated ERK1/2, and anti-ERK1/2 antibodies (FIG. 21E).

As a result, as shown in FIGS. 21A to 21C, it was confirmed that, after 9 months, tumor started to be formed in the NDRG3-overexpressing transgenic mice (FIG. 21A), and that tumor was found in various organs such as lungs, intestines, and hypogastria (FIG. 21B). Also, it was confirmed that lymphoma-expressing B cells and T cells were observed in secondary lymphoid organs such as mesenteric lymph nodes and spleens as well as the livers from the NDRG3-overexpressing mice (FIG. 21C).

Also, as shown in FIGS. 21D and 21E, it was confirmed that the hepatocellular carcinoma markers and cell proliferation markers were expressed at a significantly high level in all the three NDRG3-overexpressing transgenic mice (FIG. 20D), and the mRNA expression of the angiogenic marker IL-1α, IL-1β, IL-6, COX-2 and PAI-1 at molecular levels increased, and the ERK1/2 phosphorylation also increased (FIG. 21E). Based on the results, it was revealed that the NDRG3 promoted tumorigenesis and angiogenesis in a histological aspect.

<10-2> Confirmation of Expression of NDRG3 and Activation of Kinase Pathway in Cancer Patient

To determine the clinical relevance of the NDRG3 in cancer, a tissue microarray assay was performed using liver cancer tissues from a human liver cancer patient.

Specifically, tissue samples extracted from patients suffering of pathologically defined HCC were kindly provided by the Inje University Paik Hospital. All the tissue samples were fixed in 10% buffered formalin, and embedded in paraffin. Therefore, the paraffin-embedded HCC tissue samples (donor block) were subjected to core tissue biopsy (Diameter: 2 mm), and then aligned on new recipient paraffin blocks (tissue array blocks) using a trephine tool (Superbiochips Laboratories, Seoul, Korea). As a result, the tissue array blocks included 104 HCC tissues and 20 non-neoplastic liver issues. Four-μm sections of the tissue array blocks were subjected to immunohistological staining according to the method disclosed in Example 11-1. To detect the NDRG3 or phosphorylated ERK1/2, the sections were labeled with anti-NDRG3 and anti-phosphorylated ERK1/2 antibodies. Also, general saline was used as the negative control. Membranous staining for samples and/or NDRG3 showing a medium level (>10%) of cytoplasmic staining, and nuclear staining for phosphorylated ERK with a low level of >10% were positively graded to scores. The statistical significance between expression levels of the NDRG3 protein and phosphorylated ERK was evaluated using a chi-square (χ²) test (FIG. 22).

As a result, as shown in FIG. 22, it was confirmed that the NDRG3 protein was hardly found in normal livers, but the NDRG3 protein was strongly expressed in the cytoplasm and plasma membranes in the case of the livers from the HCC patients. In this case, it was revealed that the expression of the phosphorylated ERK1/2 protein was significantly increased. In particular, it was confirmed that, among the 25 HCC tissues expressing the NDRG3 protein, the ERK1/2 protein was phosphorylated in 19 HCC tissues (76%) (FIG. 22). Accordingly, based on the results, it was revealed that abnormal expression of the NDRG3 promoted tumorigenesis, and activated an ERK1/2 pathway.

### <Example 11> Confirmation of Regulatory Mechanism of NDRG3 in Hypoxic Response

Based on the results of Examples 3 to 11, a graphical model for the role of the NDRG3 in hypoxic responses was completed.

As shown in FIG. 23, it was confirmed that the accumulation of the HIF-1α protein was induced due to the inactivity of PHD2 at the beginning of hypoxic condition so that genes (LDHA, PDK1, etc.) involved in metabolic adaptation of cells in response to hypoxia were up-regulated to activate glycolysis. Thereafter, the NDRG3 protein is increasingly expressed by lactate generated/accumulated by the increased glycolysis as well as inhibition of hydroxylation of 294^{th} proline that is a hypoxic target site of NDRG3 due to the PHD2 inactivity under a hypoxic condition. The increasingly expressed NDRG3 served as a scaffold protein in a sustained hypoxic response to bind to c-Raf and RACK1, and the bound RACK1 recruited PKC-β proteins to form complexes. The c-Raf and ERK1/2 were then phosphorylated by PKC to activate a c-Raf-ERK pathway, thereby promoting cell proliferation and angiogenesis (see FIG. 23).

Preparative examples for the composition of the present invention are presented, as follows.

### <Preparative Example 1> Preparation of Pharmaceutical Preparations

<1-1> Preparation of Powder

| | |
|---|---|
| NDRG3 expression or activity inhibitor | 2 g |
| Lactose | 1 g |

The components were mixed, and then filled in an airtight pack to prepare a powder.

<1-2> Preparation of Tablet

| | |
|---|---|
| NDRG3 protein expression or activity inhibitor | 100 mg |
| Corn starch | 100 mg |
| Lactose | 100 mg |
| Magnesium stearate | 2 mg |

The components were mixed, and then tablet-pressed to prepare a tablet according to a conventional method of preparing a tablet.

<1-3> Preparation of Capsule

| | |
|---|---|
| NDRG3 protein expression or activity inhibitor | 100 mg |
| Corn starch | 100 mg |
| Lactose | 100 mg |
| Magnesium stearate | 2 mg |

The components were mixed, and then filled in a gelatin capsule to prepare a capsule according to a conventional method of preparing a capsule.

<1-4> Preparation of Pill

| | |
|---|---|
| NDRG3 protein expression or activity inhibitor | 1 g |
| Lactose | 1.5 g |
| Glycerin | 1 g |
| Xylitol | 0.5 g |

The components were mixed, and pills were then prepared with 4 g per pill according to a conventional method.

<1-5> Preparation of Granule

| | |
|---|---|
| NDRG3 protein expression or activity inhibitor | 150 mg |
| Soybean extract | 50 mg |
| Glucose | 200 mg |
| Starch | 600 mg |

The components were mixed, and 100 mg of 30% ethanol was added to the resulting mixture. Thereafter, the mixture was dried at 60° C. to prepare granules, which were then filled in packs.

1. A method of preventing or treating a cancer comprising: administering a pharmaceutically effective amount of a N-myc downstream-regulated gene 3 (NDRG3) protein expression or activity inhibitor to a subject in need thereof.

2. The method according to claim 1, wherein the NDRG3 protein consists of an amino acid sequence set forth in SEQ ID NO: 1.

3. The method according to claim 1, wherein the NDRG3 protein expression inhibitor is selected from the group consisting of antisense nucleotide, short interfering RNA and short hairpin RNA sequences, which complementarily bind to mRNA of an NDRG3 gene.

4. The method according to claim 1, wherein the NDRG3 protein expression inhibitor promotes hydroxylation of a proline residue at position 294 of the NDRG3 protein.

5. The method according to claim 1, wherein the NDRG3 protein expression inhibitor promotes binding of PHD2 to one or more PHD2 docking sites selected from the group consisting of 47^{th} arginine, 66^{th} asparagine, 68^{th} lysine, 69^{th} serine, 72^{nd} asparagine, 73^{rd} alanine, 76^{th} asparagine, 77^{th} phenylalanine, 78^{th} glutamic acid, 81^{st} glutamine, 97^{th} glutamine, 98^{th} glutamine, 99^{th} glutamic acid, 100^{th} glycine, 101^{st} alanine, 102^{nd} proline, 103^{rd} serine, 203^{rd} leucine, 204^{th} aspartic acid, 205^{th} leucine, 208^{th} threonine, 209^{th} tyrosine, 211^{th} methionine, 212^{th} histidine, 214^{th} alanine, 215^{th} glutamine, 216^{th} aspartic acid, 217^{th} isoleucine, 218^{th} asparagine, 219^{th} glutamine, 296^{th} valine, 297^{th} valine, 298^{th} glutamine, 300^{th} glycine, and 301^{st} lysine of the NDRG3 protein.

6. The method according to claim 1, wherein the NDRG3 protein expression inhibitor inhibits binding of lactate to 62^{nd} aspartic acid, 138^{th} glycine, 139^{th} alanine, or 229^{th} tyrosine which is a lactate binding site of the NDRG3 protein.

7. The method according to claim 1, wherein NDRG3 protein activity inhibitor is an aptamer or antibody that complementarily binds to the NDRG3 protein.

8. The method according to claim 1, wherein the NDRG3 protein activity inhibitor inhibits a binding degree of NDRG3 to one or more selected from PKC-β, RACK1 and c-Raf.

9. The method according to claim 1, wherein the cancer is selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer.

10. The method according to claim 1, further comprising administering a pharmaceutically effective amount of a HIF inhibitor to the subject.

11. A method of preventing or treating an inflammatory disease comprising: administering a pharmaceutically effective amount of a NDRG3 protein expression or activity inhibitor to a subject in need thereof.

12. The method according to claim 11, wherein the NDRG3 protein consists of an amino acid sequence set forth in SEQ ID NO: 1.

13. The method according to claim 11, wherein the NDRG3 protein expression inhibitor is selected from the group consisting of antisense nucleotide, short interfering RNA and short hairpin RNA sequences, which complementarily bind to mRNA of an NDRG3 gene.

14. The method according to claim 11, wherein the NDRG3 protein expression inhibitor promotes hydroxylation of a proline residue at position 294 of the NDRG3 protein.

15. The method according to claim 11, wherein the NDRG3 protein expression inhibitor promotes binding of PHD2 to one or more PHD2 docking sites selected from the group consisting of 47th arginine, 66th asparagine, 68th lysine, 69th serine, 72nd asparagine, 73rd alanine, 76th asparagine, 77th phenylalanine, 78th glutamic acid, 81st glutamine, 97th glutamine, 98th glutamine, 99th glutamic acid, 100th glycine, 101st alanine, 102nd proline, 103rd serine, 203rd leucine, 204th aspartic acid, 205th leucine, 208th threonine, 209th tyrosine, 211th methionine, 212th histidine, 214th alanine, 215th glutamine, 216th aspartic acid, 217th isoleucine, 218th asparagine, 219th glutamine, 296th valine, 297th valine, 298th glutamine, 300th glycine, and 301st lysine of the NDRG3 protein.

16. The method according to claim 11, wherein the NDRG3 protein expression inhibitor inhibits binding of lactate to 62nd aspartic acid, 138th glycine, 139th alanine, or 229th tyrosine which is a lactate binding site of the NDRG3 protein.

17. The method according to claim 11, wherein NDRG3 protein activity inhibitor is an aptamer or antibody that complementarily binds to the NDRG3 protein.

18. The method according to claim 11, wherein the NDRG3 protein activity inhibitor inhibits a binding degree of NDRG3 to one or more selected from PKC-β, RACK1 and c-Raf.

19. The method according to claim 11, wherein the cancer is selected from the group consisting of cervical cancer, renal cancer, gastric cancer, liver cancer, prostate cancer, breast cancer, brain tumor, lung cancer, uterine cancer, colorectal cancer, bladder cancer, blood cancer, and pancreatic cancer.

20. The method according to claim 11, wherein further administering a pharmaceutically effective amount of a HIF inhibitor to the subject.

21. An antibody or an immunologically active fragment thereof specifically binding to an N-myc downstream-regulated gene 3 (NDRG3) epitope consisting of an amino acid sequence set forth in SEQ ID NO: 3.

22. The antibody or immunologically active fragment thereof according to claim 21, wherein the antibody is selected from the group consisting of a polyclonal antibody, a monoclonal antibody, a murine antibody, a chimeric antibody, and a humanized antibody.

23. The antibody or immunologically active fragment thereof according to claim 21, wherein the immunologically active fragment is selected from the group consisting of Fab, Fab′, F(ab′)2, Fv, Fd, single-chain Fv (scFv), and disulfide-stabilized Fv (dsFv).

24-49. (canceled)


SEQUENCE LISTING (26 sequences)

SEQ ID NO: 1 - DNA, 1128 bp, Homo sapiens
atggatgaacttcaggatgttcagctcacagagatcaaaccacttctaaatgataagaatggtacaagaaacttccaggactttgactgtcaggaacatgatatagaaacaactcatggtgtggtccacgtcactataagaggcttacccaaaggaaacagaccagttatactaacatatcatgacattggcctcaaccataaatcctgtttcaatgcattctttaactttgaggatatgcaagagatcacccagcactttgctgtctgtcatgtggatgccccaggccagcaggaaggtgcaccctctttcccaacagggtatcagtaccccacaatggatgagctggctgaaatgctgcctcctgttcttacccacctaagcctgaaaagcatcattggaattggagttggagctggagcttacatcctcagcagatttgcactcaaccatccagagcttgtggaaggccttgtgctcattaatgttgacccttgcgctaaaggctggattgactgggcagcttccaaactctctggcctgacaaccaatgttgtggacattattttggctcatcactttgggcaggaagagttacaggccaacctggacctgatccaaacctacagaatgcatattgcccaagacatcaaccaagacaacctgcagctcttcttgaattcctacaatggacgcagagacctggagatcgaaagacccatactgggccaaaatgataacaaatcaaaaacattaaagtgttctactttactggtggtaggggacaattcgcctgcagttgaggctgtggtcgaatgcaattcccgcctgaaccctataaatacaactttgctaaagatggcggactgtgggggactgccccaggtagttcagcctgggaagctcaccgaggccttcaagtactttttgcagggaatgggctacataccatctgccagcatgactcggctcgcccgatcacgaacccactcaacctcgagtagcctcggctctggagaaagtcccttcagccggtctgtcaccagcaatcagtcagatggaactcaagaatcctgtgagtcccctgatgtcctggacagacaccagaccatggaggtgtcctgctaa

SEQ ID NO: 2 - protein, 284 aa, Homo sapiens
EHDIETTHGVVHVTIRGLPKGNRPVILTYHDIGLNHKSCFNAFFNFEDMQEITQHFAVCHVDAPGQQEGAPSFPTGYQYPTMDELAEMLPPVLTHLSLKSIIGIGVGAGAYILSRFALNHPELVEGLVLINVDPCAKGWIDWAASKLSGLTTNVVDIILAHHFGQEELQANLDLIQTYRMHIAQDINQDNLQLFLNSYNGRRDLEIERPILGQNDNKSKTLKCSTLLVVGDNSPAVEAVVECNSRLNPINTTLLKMADCGGLPQVVQPGKLTEAFKYFLQGMGY

SEQ ID NO: 3 - protein, 12 aa, Homo sapiens
QNDNKSKTLKCS

SEQ ID NO: 4 - RNA, 58 nt, Artificial Sequence
  shNDRG3
ccggacauuggccucaaccauaaaucucgagauuuaugguugaggccaauguuuuuug

SEQ ID NO: 5 - RNA, 19 nt, Artificial Sequence
  siNDRG3
cgccugaacccuauaaaua

SEQ ID NO: 6 - RNA, 19 nt, Artificial Sequence
  siNDRG3
gcuaaaggcuggauugacu

SEQ ID NO: 7 - RNA, 19 nt, Artificial Sequence
  siNDRG3
aaacagaccaguuauacua

SEQ ID NO: 8 - RNA, 19 nt, Artificial Sequence
  siNDRG3
gaucaaaccacuucuaaau

SEQ ID NO: 9 - DNA, 22 bp, Artificial Sequence
  NDRG3 forward primer
aaccataaatcctgtttcaatg

SEQ ID NO: 10 - DNA, 21 bp, Artificial Sequence
  NDRG3 reverse primer
tccacaacattggttgtcagg

SEQ ID NO: 11 - DNA, 30 bp, Artificial Sequence
  NDRG3(N66D) sense primer
gaccataaatcctgtttcaatgcattcttt

SEQ ID NO: 12 - DNA, 30 bp, Artificial Sequence
  NDRG3(N66D) antisense primer
gaggccaatgtcatgatatgttagtataac

SEQ ID NO: 13 - RNA, 21 nt, Artificial Sequence
  siGFP sense
guucagcguguccggcgaguu

SEQ ID NO: 14 - RNA, 21 nt, Artificial Sequence
  siGFP antisense
cucgccggacacgcugaacuu

SEQ ID NO: 15 - RNA, 21 nt, Artificial Sequence
  siPHD1 sense
caucgagccacucuuugacuu

SEQ ID NO: 16 - RNA, 21 nt, Artificial Sequence
  siPHD1 antisense
gucaaagaguggcucgauguu

SEQ ID NO: 17 - RNA, 21 nt, Artificial Sequence
  siPHD2 sense
aacggguuauguacgucauuu

SEQ ID NO: 18 - RNA, 21 nt, Artificial Sequence
  siPHD2 antisense
augacguacauaacccguuuu

SEQ ID NO: 19 - RNA, 21 nt, Artificial Sequence
  siPHD3 sense
ccagauaugcuaugacuguuu

SEQ ID NO: 20 - RNA, 21 nt, Artificial Sequence
  siPHD3 antisense
acagucauagcauaucugguu

SEQ ID NO: 21 - RNA, 21 nt, Artificial Sequence
  siP4HTM sense
gagugucggcucaucauccuu

SEQ ID NO: 22 - RNA, 21 nt, Artificial Sequence
  siP4HTM antisense
ggaugaugagccgacacucuu

SEQ ID NO: 23 - RNA, 21 nt, Artificial Sequence
  siP4HA1 sense
gaucuggugacuucucugauu

SEQ ID NO: 24 - RNA, 21 nt, Artificial Sequence
  siP4HA1 antisense
ucagagaagucaccagaucuu

SEQ ID NO: 25 - DNA, 27 bp, Artificial Sequence
  NDRG3(G138W) sense primer
gtttgggctggagcttacatcctcagc

SEQ ID NO: 26 - DNA, 27 bp, Artificial Sequence
  NDRG3(G138W) antisense primer
tccaattccaatgatgcttttcaggct